(19) United States
(12) Patent Application Publication
(10) Pub. No.: US 2026/0053799 A1
(43) Pub. Date: Feb. 26, 2026

(54) COMPOUNDS FOR TREATING CANCER

(71) Applicant: EVEXTA BIO, PARIS (FR)

(72) Inventors: DOMINIQUE BRIDON, PARIS (FR); HÉLÈNE SICARD, PARIS (FR); CHRISTEL NAVARRO, PARIS (FR)

(21) Appl. No.: 19/102,512

(22) PCT Filed: Aug. 11, 2023
(86) PCT No.: PCT/EP2023/072264

§ 371 Date: Feb. 10, 2025

(30) Foreign Application Priority Data
Aug. 11, 2022 (EP) 22306214.2
Mar. 30, 2023 (EP) 23305457.6

(51) Int. Cl. A61K 31/506; A61K 31/137; A61K 45/06; A61P 35/00

(52) U.S. Cl. CPC A61K 31/506; A61K 31/137; A61K 45/06; A61P 35/00

(57) ABSTRACT

The present invention relates to a quinazoline carboxamide azetidine compound, and to pharmaceutical combinations and compositions comprising such a compound preferably together with at least one distinct therapeutic agent, preferably anti-cancer agent, as well as to uses thereof for treating a disease, preferably a cancer, even more preferably estrogen receptor-positive (ER+) cancer, in particular in a subject resistant to endocrinal therapy.

The present invention relates to field of oncology. It more particularly relates to a quinazoline carboxamide azetidine compound, and to pharmaceutical combinations and compositions comprising such a compound preferably together with at least one distinct therapeutic agent, preferably anti-cancer agent, as well as to uses thereof for treating a disease, preferably a cancer, even more preferably estrogen receptor-positive (ER*) cancer, in particular in a subject resistant to hormonal therapy, even more preferably in a subject suffering of breast cancer and resistant to hormonal therapy.

### BACKGROUND OF THE INVENTION

Breast cancer is the most prevalent cancer worldwide with 2.3 million new diagnoses and 685,000 deaths globally in 2020. Hormone receptor positive (HR+) breast cancer is the most common subtype of breast cancers accounting for around 70% of all worldwide breast cancer population.

Approximately 80% of the HR+ breast cancers in women are estrogen receptor positive (“ER+” or “ER− positive” status) and/or progesterone receptor positive (“PR+” or “PgR positive”). Similarly, 90% of breast cancers in men are ER positive.

Around 81% of breast cancers are invasive, or infiltrating, i.e., metastatic. In other words, the abnormal cells have broken through the walls of the glands or ducts where they originated and grown into surrounding tissues. Although breast cancer was historically referred to as a single disease, it is now considered a group of diseases, consisting of four major molecular subtypes and at least 21 distinct histological subtypes (types of tissue in which the cancer originates) that differ in risk factors, presentation, response to treatment, and outcomes. For example, around 5 to 10% of breast cancer patients will present with metastatic disease at the time of diagnosis and the incidence of breast cancer brain metastases (“BCBMs”) in estrogen receptor positive breast cancers is 14%, with a median overall survival after the development of brain metastases of 9-10 months (Brosnan et al., 2018).

The importance and role of the estrogen receptor (ER) pathway have been well-recognized in both breast cancer development and progression. The physician's choice Standard of Care (SOC) treatment of estrogen receptor-positive breast cancer are endocrine therapies (also called “hormone therapy”, “hormonal therapy” or “hormone treatment”) with the common being: selective estrogen receptor modulators (“SERM”) such as tamoxifen and “tamoxifen-like” compounds for example, 4 hydroxytamoxifen, endoxifen, toremifene, droloxifene, idoxifene, raloxifene, arzoxifene, bazedoxifene, pipindoxifene or lasofoxifene; aromatase inhibitors (“AI”) such as for example aminoglutethimide, anastrozole, exemestane, fadrozole, letrozole, vorozole or AZD9496; selective estrogen receptor degraders or down-regulators (“SERD”) such as for example amcenestrant, azenosertib (ZN-c5), borestrant, brilanestrant, camizestrant, elacestrant, fulvestrant, giredestrant, imlunestrant, rintodestrant AZD9496 (LSZ102), D-0502, LY3484356, GDC-0927 or SHR9549; complete estrogen receptor antagonist (CERAN) such as OP-1250; and luteinizing hormone-releasing hormone (LHRH) and/or gonadotropin releasing hormone (GnRH) agonists such as buserelin, cetrorelix, degarelix, gonadorelin, goserelin, leuprolide, triptorelin and triptorelix.

Unfortunately, resistance develops in 30-50% of patients treated with endocrine therapies due to a sophisticated and at times redundant interference at the molecular level between growth factors, the ER, and downstream cell-signaling pathways. Several mechanisms of resistance to endocrine therapies have been described (Patel et al., 2018) such as alterations in ER and/or in the ER pathway, activation of the PI3K/Akt/mTOR (“PAM”) pathway, activation of growth factor receptor pathway(s) [involving the epidermal growth factor receptor (EGFR), fibroblast growth factor receptor (FGFR), insulin growth factor receptor (IGFR) and/or vascular endothelial growth factor receptor (VEGFR)], alterations in the cell cycle, alterations in the ubiquitin-proteasome pathway or increased activity of the bromodomain and/or extra-terminal domain of proteins. These multiple resistance mechanisms to endocrine therapies make the strategy to treat refractory disease challenging and dependent on the patient context.

For example, the deregulated activation of the PI3K/Akt/mTOR pathway, which plays a key role in proliferation and survival under normal conditions, is an adaptive mechanism of treatment resistance in ER+ breast cancer and can correlate with a poorer outcome in patients treated with endocrine therapies.

In fact, PI3K, Akt and downstream effectors of mTOR can phosphorylate and activate ER in the absence of estrogen, thereby conferring resistance to endocrine therapies. Furthermore, some studies suggest an inverse relationship between PI3K activation and ER expression. In fact, gain-of-function mutations in PI3K occur in over 30% of ER+ breast cancers, underscoring the role of this pathway in this setting. These findings have led to combination strategies that include the administration of an endocrine/hormonal agent, more preferably a SERD such as for example fulvestrant or an AI such as exemestane, together with an inhibitor of the PI3K/Akt/mTOR pathway such as buparlisib (BKM120), pilaralisib (XL147, SAR245408), pictilisib (GDC-0941), sonolisib (PX-866), dactolisib (BEZ235), sapanisertib (INK128, MLN0128), voxtalisib (XL765, SAR245409), serabelisib (MLN1117), alpelisib (BYL719), perifosine (KRX-0401), MK2206, ipatasertib (GDCO0068), GSK690693, temsirolimus (CCI-779), ridaforolimus (MK8669; deforolimus), sirolimus (rapamycin), everolimus (RAD001), AZD-8055 or OSI-027 (ASP7486).

Another example is the alterations in the cell cycle. During a quiescent state, tumor suppressor retinoblastoma (Rb) protein remains hypophosphorylated and bound to transcription factor E2F, preventing E2F from facilitating the transcription of genes involved in the G1-S phase progression of the cell cycle. Upon growth factor stimulation, Rb is phosphorylated by cyclin-dependent kinases 4 and 6 (CDK4/6) that are in a complex with Cyclin D, resulting in the release of E2F and progression through the cell cycle. Several lines of evidence link these cell cycle regulators to the ER pathway, including the observation that CDK4/6 are often hyperactivated. These findings have led to combination strategies that include an endocrine agent more preferably a SERD such as fulvestrant and an inhibitor of CDK4/6 (CDK4/6i) such as palbociclib (PD0332991), ribociclib (LEE011) or abemaciclib (LY2835219).

A further example is the activation of growth factor receptor pathways, for example through the overexpression or amplification of the Human Epidermal Growth Factor Receptor-2 (Her2), a member of the epidermal growth factor receptor (EGFR) family, that can confer resistance to endocrine agents. The addition of a dual EGFR/Her2 targeting agent such as lapatinib (GW572016) to a SERD such as fulvestrant was evaluated in patients with ER+ advanced breast cancer but no statistically relevant clinical therapeutic effect has been observed on ER+/HER2+ patients (Burstein et al., 2014).

Tumor response is heightened with adjuvant therapy that includes an mTORC1 inhibitor (everolimus), CDK4/6 inhibitors (palbociclib/ribociclib/abemaciclib), and an α isoform-specific PI3K inhibitor (alpelisib).

However, about 25% of ER+ breast cancers patients with primary disease and almost all patients with metastases will present with or eventually develop endocrine resistance.

Several studies have demonstrated that ER and more especially the functional Estrogen Receptor 1 (“ESR1”) is a key driver of endocrine resistance during the progression of ER+ breast cancer. For example, ER expression is positively correlated with tamoxifen outcome and alterations in ER and the ER pathway have been described as resulting in treatment resistance to SERMs such as tamoxifen or SERDs such as fulvestrant. Furthermore, ER loss through epigenetic silencing, via DNA methylation or deacetylation of histones, has been described as a mechanism of resistance to both compounds (Fan et al., 2006; Parl, 2003; Yang, Phillips, Ferguson, et al., 2001). In particular, it has been described that ER loss is heavily correlated with de novo resistance versus acquired resistance to tamoxifen, underscoring the importance of early target engagement and inhibition in this setting. Several clinical trials are examining the combination of endocrine therapy with epigenetic modifiers such as mutations in the ligand-binding domain (LBD) of the ERa protein (Estrogen Receptor alpha), encoded by the Estrogen Receptor 1 gene (herein identified as ESR1). These mutations in the gene encoding ERa, change the conformation of the ERa protein, increase its interaction with its co-activators, promote a constitutive active form of the receptor in absence of hormone, and assist tumor cells in evading hormonal treatment. In general, the absence of detectable ESR1 mutations in patients with treatment-naive disease, and the correlation between the frequency of patients with tumors harboring these mutations and the number of endocrine treatments received, suggest that, under selective treatment pressure, clonal expansion of rare mutant clones occurs, leading to resistance.

Fifty five next generation endocrine agents are in clinical stage development for the management of ER+ breast cancer (Maxwell R. Lloyd et al., 2022) as for example a CERAN agent which is a tetrahydro-1H-pyrido[3,4-b]indole compound as described in WO2017059139. However, several other candidate compounds showing preclinical antitumor activity have also been discontinued during various stages of clinical testing such as brilanestrant (GDC0810), an oral SERD described in US20150258080 and US2015258099, that was removed from development after a phase II clinical trial and which failed to demonstrate comparable or superior efficacy to fulvestrant.

There is a need to develop rationale-based novel therapeutic strategies for patients developing endocrine treatment resistance to make them responder again to endocrine treatment.

A novel quinazoline carboxamide azetidine compound, 4-[(S)-2-Azetidin-1-yl-1-(4-chloro-3-trifluoromethyl-phenyl)-ethylamino]-quinazoline-8-carboxylic acid amide (M2698), was previously described in WO201269146 as a potent orally bioavailable, selective inhibitor of p70S6K, AKT1 and AKT3 that affects tumor growth in mouse models of cancer and crosses the blood-brain barrier (Machl et al., 2016). M2698 treatment was well tolerated as monotherapy and combined with trastuzumab or tamoxifen in patients with ER+ and HER2+breast cancer, respectively. Overall, the efficacy of M2698 monotherapy was modest in this patient population (Tsimberidou et al., 2021).

Inventors now reveal for the first time, that the administration of a quinazoline carboxamide azetidine compound, preferably of 4-[(S)-2-Azetidin-1-yl-1-(4-chloro-3-trifluoromethyl-phenyl)-ethylamino]-quinazoline-8-carboxylic acid amide, makes possible to sensitize again the ER+ cancerous tumors identified as resistant to endocrine therapy to cancer treatment, including endocrine therapy, thereby substantially improving treatment outcomes.

### BRIEF SUMMARY

Endocrine therapy (also called “hormone therapy”, “hormonal therapy” or “hormone treatment”) has been successful at improving progression-free survival (“PFS”) and overall survival (“OS”) of patients. However, the development of resistance to endocrine and/or ER treatments remains a roadblock in ER+ cancers, especially in breast cancer. Intrinsic resistance to endocrine therapies explains that only 30% of patients with metastatic disease see initial tumor regression with endocrine therapies, where, in almost all patients the resistance develops eventually, and tumors frequently recur. Moreover, more than 20% of patients with early breast cancer will develop endocrine resistance throughout treatment. Resistance to endocrine therapies has been associated with mutations in the Estrogen Receptor 1 (ESR1) gene, alterations in receptor tyrosine kinases such as HER2, and alterations in signaling pathways such as the MAPK pathway.

Thus, there is a need to develop new therapeutic strategies, effective to treating in particular estrogen receptor positive (ER+) tumors, in particular tumors resistant to endocrine therapy, preferably tumors harboring mutations in the Estrogen Receptor 1 (ESR1) gene encoding for the Estrogen Receptor alpha (ERα, ERa or ESR1) protein in patients, and in particular breast cancer patients, who have developed resistance to endocrine therapy or who are at risk of developing endocrinal resistance (Sung Qwe Ahn et al., 2022).

The present invention now provides such new and advantageous therapeutic strategies.

Inventors more particularly herein describe a quinazoline carboxamide azetidine compound, a combination involving, in particular consisting of, at least two compounds one of which is a quinazoline carboxamide azetidine compound, or a composition, in particular a pharmaceutical composition, comprising a quinazoline carboxamide azetidine compound and a pharmaceutically acceptable carrier. The combination or composition, in addition to the quinazoline carboxamide azetidine compound, preferably comprises in addition a distinct therapeutic agent, preferably an anti-cancer agent, more preferably an antineoplastic agent and/or a signal transduction inhibitor. Inventors also describe said combination or composition for use as a medicament, preferably for treating a hormone dependent disease, even more preferably for treating cancer, in particular for treating estrogen receptor-positive (ER+) cancer, in a subject in need thereof.

The quinazoline carboxamide azetidine compound is advantageously a quinazoline carboxamide azetidine compound of formula (I):

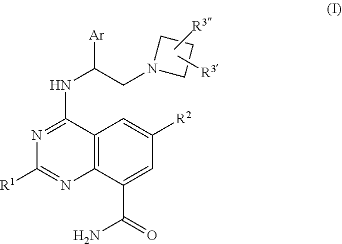

and/or pharmaceutically acceptable polymorphs, enantiomers, stereoisomers, salts, solvates or tautomers thereof, including mixtures thereof in all ratios, wherein:
R¹ is H or LA;
R² is Hal, O(LA), N(LA)(LA)′, CONH(LA), Ar, CONH₂ or A;
R^{3′}, R^{3″} independently are H, LA or Hal;
Ar is a mono- or bicyclic aromatic homo- or heterocycle having 0, 1, 2, 3 or 4 N, O and/or S atoms and 5, 6, 7, 8, 9, or 10 skeleton atoms, which may be unsubstituted or, independently of one another, mono, di- or trisubstituted by Hal, A, Art, OH, SH, OA, O(Ar1), NH₂, NHA, NH(Ar1), NA₂>NO₂, CN, OCN, SCN, COOH, COOA, CONH₂, CONHA, CONH(Art), CONA₂, NHCOA, NHCO(Art), NHCONHA, NHCONH(Art), NHCONH₂, NHSO₂A, NHSO₂(Ar1), COA, CO(Ar1), SO₂NH₂, SO₂A, SO₂(Ar1) and/or SO₂Hal, and in which a ring N-atom may be substituted by an O-atom to form an N-oxide group, and in which in the case of a bicyclic aromatic cycle on one of the two rings may be partly saturated; Ar is a monocyclic aromatic homo- or heterocycle having 0, 1, 2 or 3 N, O and/or S atoms and 5 or 6 skeleton atoms, which may be unsubstituted or, independently of one another, mono-, di- or trisubstituted by Hal, LA, OH, SH, O(LA), NH₂, NH(LA), N(LA)₂, NO₂, CN, OCN, SCN, COOH, COO(LA), CONH₂, CONH(LA), CON(LA)₂, NHCO(LA), CHO, CO(LA), SO₂NH₂, SO₂(LA) and/or SO₂Hal;
A is unbranched or branched linear or cyclic alkyl having 1, 2, 3, 4, 5, 6, 7 or 8 C atoms, in which one or two CH₂ groups may be replaced by an O or S atom and/or by an —NH—, —CO—, —NHCOO—, —NHCONH—, —N(LA)-, —CONH—, —NHCO— or —CH═CH— group, and in which 1-3H atoms may be replaced by Hal, and in which one or two CH₃ groups may be replaced by OH, SH, NH₂, NH(LA), N(LA)₂, NHCOOH, NHCONH₂ or CN;
LA is unbranched or branched, linear alkyl having 1, 2, 3 or 4 C atoms, wherein 1, 2 or 3H atoms may be replaced by Hal, e.g., methyl, ethyl, trifluoromethyl, difluoromethyl, 1,1,1-trifluoroethyl, propyl, isopropyl, butyl, isobutyl, sec-butyl or tert-butyl; and
Hal is F, Cl or Br, preferably F or Cl, most preferably F;
or a pharmaceutically acceptable polymorph, enantiomer, stereoisomer, salt, solvate or tautomer thereof, or any mixture thereof in all ratios.

The quinazoline carboxamide azetidine compound is preferably 4-[(S)-2-Azetidin-1-yl-1-(4-chloro-3-trifluoromethyl-phenyl)-ethylamino]-quinazoline-8-carboxylic acid amide (also herein identified as “M2698”) or a pharmaceutically acceptable polymorph, enantiomer, stereoisomer, salt, solvate or tautomer thereof, or any mixture thereof in all ratios.

The antineoplastic agent is preferably an hormonal therapeutic agent or a chemotherapeutic agent.

The chemotherapeutic agent can be selected for example from an alkylating agent, a platinum coordination complex, a cytotoxic antibiotic, an antimetabolite, a taxane, a topoisomerase inhibitor and a vinca alkaloid.

The antineoplastic agent is preferably an hormonal therapeutic agent (also herein identified as an “hormone therapeutic agent” or “endocrine therapeutic agent”), even more preferably an antiestrogen agent. It is for example selected from a Selective ER down-regulator Degrader (SERD), a Selective ER Modulator (SERM), an Aromatase Inhibitor (AI), a Complete Estrogen Receptor Antagonist (CERAN), a luteinizing hormone-releasing hormone (LHRH), a gonadotropin releasing hormone (GnRH) agonist, and any mixture thereof.

In a particular embodiment, the therapeutic agent or signal transduction inhibitor is not a HER2 inhibitor, a HER3 inhibitor or a HER3 nanobody.

In a particular aspect, the HER2 inhibitor is not lapatinib or trastuzumab.

In a particular aspect, the HER3 inhibitor is not MM-121 [i.e., a fully humanized anti-Her3 antibody that specifically blocks the binding of HRG1-(a neuregulin-1 type I polypeptide) to Her3], MM-11 [a bispecific antibody binding to two different target proteins: ErbB2 and ErbB3] or U3-1287 (also identified as AMG888, the first fully humanized Her3 monoclonal antibody).

In a particular embodiment, the combination or composition of the invention comprises (a) a quinazoline carboxamide azetidine compound, preferably a compound of formula (I) such as 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, or any mixture thereof in all ratios (b) a distinct therapeutic agent and optionally (c) an anti-cancer agent, with the proviso that if one of the compound (b) or (c) is a HER2 inhibitor, said HER2 inhibitor is not lapatinib or trastuzumab.

In a particular embodiment, the combination or composition of the invention comprises (a) a quinazoline carboxamide azetidine compound, preferably a compound of formula (I) such as 4-(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, or any mixture thereof in all ratios (b) a distinct therapeutic agent and, optionally (c) an anti-cancer agent, with the proviso that if one of the compound (b) or (c) is a HER3 inhibitor, said HER3 inhibitor is not MM-121, MM-11 or U3-1287.

The signal transduction inhibitor is preferably a cyclin-dependent kinase (CDK) inhibitor selected for example from a CDK 1, 2, 4, 5, 6 and/or 7 inhibitor and any mixture thereof, more preferably a CDK4/6 inhibitor.

The treatment may involve the administration of an additional anti-cancer agent selected for example from an anti-angiogenic agent, a signal transduction inhibitor, an antineoplastic agent, a therapeutic antibody or a (functional) fragment thereof (including a single chain antibody), an antibody-drug conjugate, a small molecule, a growth factor receptor agent, an antisense molecule, and any mixture thereof.

In a particular embodiment, the combination or composition of the invention comprises (a) a quinazoline carboxamide azetidine compound, preferably a compound of formula (I) such as 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, or any mixture thereof in all ratios and at least one anti-cancer agent, with the proviso that said anti-cancer agent is not a MEK inhibitor, in particular a MEK inhibitor selected from trametinib, cobimetinib, selumetinib, refametinib and pimasertib. Preferably the MEK inhibitor is not pimasertib.

In another particular embodiment, the combination or composition of the invention comprises (a) a quinazoline carboxamide azetidine compound, preferably a compound of formula (I) such as 4-(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, or any mixture thereof in all ratios and at least one anti-cancer agent, with the proviso that said anti-cancer agent is not an EGFR inhibitor, in particular an EGFR inhibitor selected from gefitinib, erlotinib, afatinib, brigatinib, icotinib, osimertinib and cetuximab. Preferably the EGFR inhibitor is not cetuximab. In a further particular embodiment, the combination or composition of the invention comprises (a) a quinazoline carboxamide azetidine compound, preferably a compound of formula (I) such as 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, or any mixture thereof in all ratios (b) a distinct therapeutic agent and, optionally (c) an anti-cancer agent, with the proviso that if the distinct therapeutic agent (b) or anti-cancer agent (c) is a MEK inhibitor, for example trametinib, cobimetinib, selumetinib, refametinib or pimasertib, in particular pimasertib, then the other (b) or (c) compound is not an EGFR inhibitor, for example gefitinib, erlotinib, afatinib, brigatinib, icotinib, osimertinib or cetuximab, in particular cetuximab, or if the distinct therapeutic agent (b) or anti-cancer agent (c) is an EGFR inhibitor, for example cetuximab, then the other (b) or (c) compound is not a MEK inhibitor, for example pimasertib.

In a particular embodiment, the combination or composition of the invention comprises (a) a quinazoline carboxamide azetidine compound, preferably a compound of formula (I) such as 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof or any mixture thereof in all ratios and (b) a distinct therapeutic agent selected from an antineoplastic agent and a signal transduction agent and, optionally (c) an additional distinct anti-cancer agent.

In a more particular embodiment, the combination or composition of the invention comprises (a) a quinazoline carboxamide azetidine compound, preferably a compound of formula (I) such as 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof or any mixture thereof in all ratios, (b) a distinct therapeutic agent selected from an antineoplastic agent, an hormonal therapeutic agent for example selected from a SERD, a SERM, an AI, a CERAN, a LHRH, and a GnRH agonist, preferably a SERD for example amcenestrant, azenosertib (ZN-c5), borestrant, brilanestrant, camizestrant, elacestrant, fulvestrant, giredestrant, imlunestrant, rintodestrant AZD9496, D-0502, LY3484356, GDC-0927 or SHR9549, and, optionally (c) an additional distinct anti-cancer agent.

In a particular aspect, inventors herein describe a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a Selective ER down-regulator Degrader (SERD), preferably elacestrant or a pharmaceutically acceptable polymorph, enantiomer, stereoisomer, salt, solvate or tautomer thereof, and a composition comprising the combination of (a) and (b) and a pharmaceutically acceptable carrier.

Also herein described is a method of treating a hormone dependent disease in a subject, wherein the method comprises administering a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a Selective ER down-regulator Degrader (SERD), preferably elacestrant or a pharmaceutically acceptable polymorph, enantiomer, stereoisomer, salt, solvate or tautomer thereof, or a composition comprising the combination of (a) and (b) and a pharmaceutically acceptable carrier, to the subject, thereby treating the subject.

Further herein described is a kit comprising (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a Selective ER down-regulator Degrader (SERD), preferably elacestrant or a pharmaceutically acceptable polymorph, enantiomer, stereoisomer, salt, solvate or tautomer thereof, in different containers, or a composition comprising the combination of (a) and (b) and a pharmaceutically acceptable carrier, and material(s) for administering M2698 and/or the SERD or for administering the composition.

In a more particular embodiment, the combination or composition of the invention comprises (a) a quinazoline carboxamide azetidine compound, preferably a compound of formula (I) such as 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof or any mixture thereof in all ratios, and (b) a distinct therapeutic agent selected from a signal transduction inhibitor, preferably a cyclin-dependent kinase (CDK) inhibitor for example selected from a CDK4/6 inhibitor such as abemaciclib, palbocilcib and ribociclib, and, optionally (c) an additional distinct anti-cancer agent.

In another particular aspect, inventors herein describe a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a cyclin-dependent kinase (CDK) 4/6 inhibitor, preferably abemaciclib or a pharmaceutically acceptable polymorph, enantiomer, stereoisomer, salt, solvate or tautomer thereof, or a composition comprising the combination of (a) and (b) and a pharmaceutically acceptable carrier.

Also herein described is a method of treating a hormone dependent disease in a subject, wherein the method comprises administering a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a cyclin-dependent kinase (CDK) 4/6 inhibitor, preferably abemaciclib or a pharmaceutically acceptable polymorph, enantiomer, stereoisomer, salt, solvate or tautomer thereof, or a composition comprising the combination of (a) and (b) and a pharmaceutically acceptable carrier, to the subject, thereby treating the subject.

Further herein described is a kit comprising (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a cyclin-dependent kinase (CDK) 4/6 inhibitor in different containers, preferably abemaciclib or a pharmaceutically acceptable polymorph, enantiomer, stereoisomer, salt, solvate or tautomer thereof, or a composition comprising the combination of (a) and (b) and a pharmaceutically acceptable carrier, and material(s) for administering M2698 and/or the CDK 4/6 inhibitor or for administering the composition.

The disease targeted in the context of the present invention is preferably a hormone dependent disease, whether malignant or non-malignant. The hormone dependent disease is preferably a pre-malignant (/pre-cancerous) disease or a malignant disease, i.e., a cancer. It is typically a hormone receptor-positive (HR⁺) cancer, for example a cancer selected from a brain cancer, breast cancer, lung cancer, ovarian cancer, peritoneal cancer, fallopian tube cancer, endometrial cancer (for example a type I endometrial cancer), uterine cancer, bladder cancer, colon cancer, prostate cancer, esophageal cancer, liver cancer, pancreatic cancer, stomach cancer and any other known HR⁺ cancer.

In a preferred aspect, the hormone receptor-positive (HR⁺) cancer is an estrogen receptor-positive (ER⁺) cancer.

The estrogen receptor-positive (ER+) cancer is preferably selected from breast cancer, in particular metastatic breast cancer, typically a metastatic breast cancer resistant and/or refractory to standard-of-care treatment, ovarian cancer, and type I endometrial cancer.

The subject in need is preferably a subject who has been treated, and possibly who is still on treatment, with a drug used in endocrine (hormonal/hormone) therapy, such as a Selective ER Modulator (SERM), a Selective ER down-regulator Degrader (SERD), an Aromatase Inhibitor (AI) inhibitor, a CERAN inhibitor, an hormone, a Luteinizing hormone-releasing hormone (LHRH) agonist, a gonadotropin releasing hormone (GnRH) agonist, a progestin, an anti-androgen, a CYP17 inhibitor, an adrenolytic agent; a cyclin-dependent kinase (CDK) inhibitor; a PI3K/AKT/mTOR (“PAM”) pathway inhibitor; or any combination thereof, in particular the combination of a CDK inhibitor and of an hormonal therapeutic agent, or the combination of a CDK inhibitor, an hormonal therapeutic agent and a PI3K/AKT/mTOR (“PAM”) pathway inhibitor. A drug used in endocrine therapy can be selected from a Selective ER Modulator (SERM), a Selective ER down-regulator Degrader (SERD), an Aromatase Inhibitor (AI) and a complete estrogen receptor antagonist (CERAN).

In a preferred embodiment, the ER+ breast cancer is characterized by a mutated Estrogen Receptor 1 (ESR1) cancerous tumor wherein the mutation (including for example an addition, a deletion, a substitution or a frame shift mutation) occurs in the ligand-binding domain (“LBD”) of the ERa sequence of SEQ ID NO: 1. In a particular aspect, the LBD-mutant forms of ER favor the agonist conformation of the receptor thereby mediating constitutive transcription leading to clinical resistance. A preferred ERa mutant comprises at least one of the following mutations: E380Q, V392I, F404fs, V422del, S463P L536H, L536P, L536Q, L536R, Y537C, Y537D, Y537S, Y537N, D538G, even more preferably the Y537S and/or D538G mutation(s).

In a particular embodiment, the quinazoline carboxamide azetidine compound, or composition comprising a quinazoline carboxamide azetidine compound, according to the invention is for use in an anti-cancer treatment involving in addition the (co-)administration to the subject of a drug selected from a Selective ER modulator (SERM), a Selective ER down-regulator (SERD), an aromatase Inhibitor (AI), a Complete Estrogen Receptor Antagonist (CERAN), an inhibitor of the cell cycle, a PI3K/Akt/mTOR (“PAM”) pathway inhibitor, and/or an inhibitor of the growth factor receptor.

In a further preferred embodiment, the quinazoline carboxamide azetidine compound, or composition comprising a quinazoline carboxamide azetidine compound, according to the invention is for use in an anti-cancer treatment involving in addition the (co-)administration to the subject of a drug selected from an antineoplastic agent (such as for example a Selective ER modulator (SERM), a Selective ER down-regulator (SERD), an aromatase Inhibitor (AI), and/or a Complete Estrogen Receptor Antagonist (CERAN)), a signal transduction inhibitor such as for example a CDK inhibitor, and a PI3K/Akt/mTOR (“PAM”) pathway inhibitor.

### DETAILED DESCRIPTION

The present invention provides novel therapeutic compounds, combinations of compounds, and pharmaceutical compositions comprising such compounds, as well as the uses thereof in human medicine, preferably in oncology.

In particular, inventors herein describe a combination of (a) a quinazoline carboxamide azetidine compound and (b) a distinct therapeutic agent, preferably an anti-cancer agent selected from an anti-angiogenic agent, a signal transduction inhibitor, an antineoplastic agent, a therapeutic antibody or a (functional) fragment thereof (including for example a single chain antibody), an antibody-drug conjugate, a small molecule, a growth factor receptor agent, an antisense molecule and any combination thereof, or a composition comprising the combination of (a) and (b) and a pharmaceutically acceptable carrier.

They also describe a quinazoline carboxamide azetidine compound, more particularly a compound of formula (I):

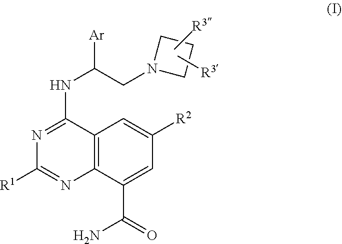

alone or in the form of any mixture of said compound with a pharmaceutically acceptable polymorph, enantiomer, stereoisomer, salt, solvate or tautomer thereof, or any mixture thereof in all ratios, wherein:
R¹ is H or LA;
R² is Hal, O(LA), N(LA)(LA)′, CONH(LA), Ar, CONH₂ or A;
R^{3′}, R^{3″} independently are H, LA or Hal;
Ar is a mono- or bicyclic aromatic homo- or heterocycle having 0, 1, 2, 3 or 4 N, O and/or S atoms and 5, 6, 7, 8, 9, or 10 skeleton atoms, which may be unsubstituted or, independently of one another, mono-, di- or trisubstituted by Hal, A, Art, OH, SH, OA, O(Ar1), NH₂, NHA, NH(Ar1), NA₂>NO₂, CN, OCN, SCN, COOH, COOA, CONH₂, CONHA, CONH(Art), CONA₂, NHCOA, NHCO(Art), NHCONHA, NHCONH(Art), NHCONH₂, NHSO₂A, NHSO₂(Ar1), COA, CO(Ar1), SO₂NH₂, SO₂A, SO₂(Ar1) and/or SO₂Hal, and in which a ring N-atom may be substituted by an O-atom to form an N-oxide group, and in which in the case of a bicyclic aromatic cycle on one of the two rings may be partly saturated; Ar is a monocyclic aromatic homo- or heterocycle having 0, 1, 2 or 3 N, O and/or S atoms and 5 or 6 skeleton atoms, which may be unsubstituted or, independently of one another, mono-, di- or trisubstituted by Hal, LA, OH, SH, O(LA), NH₂, NH(LA), N(LA)₂, NO₂, CN, OCN, SCN, COOH, COO(LA), CONH₂, CONH(LA), CON(LA)₂, NHCO(LA), CHO, CO(LA), SO₂NH₂, SO₂(LA) and/or SO₂Hal;
A is unbranched or branched linear or cyclic alkyl having 1, 2, 3, 4, 5, 6, 7 or 8 C atoms, in which one or two CH₂ groups may be replaced by an O or S atom and/or by an —NH—, —CO—, —NHCOO—, —NHCONH—, —N(LA)-, —CONH—, —NHCO— or —CH═CH— group, and in which 1-3H atoms may be replaced by Hal, and in which one or two CH₃ groups may be replaced by OH, SH, NH₂, NH(LA), N(LA)₂, NHCOOH, NHCONH₂ or CN;
LA is unbranched or branched, linear alkyl having 1, 2, 3 or 4 C atoms, wherein 1, 2 or 3H atoms may be replaced by Hal, e.g., methyl, ethyl, trifluoromethyl, difluoromethyl, 1,1,1-trifluoroethyl, propyl, isopropyl, butyl, isobutyl, sec-butyl or tert-butyl; and
Hal is F, Cl or Br, preferably F or Cl, most preferably F.

Inventors also describe a combination involving, in particular consisting of, at least two compounds one of which is a quinazoline carboxamide azetidine compound, or a composition, in particular a pharmaceutical composition comprising a quinazoline carboxamide azetidine compound and a pharmaceutically acceptable carrier.

A preferred combination is the combination of (a) a quinazoline carboxamide azetidine compound of formula (I):

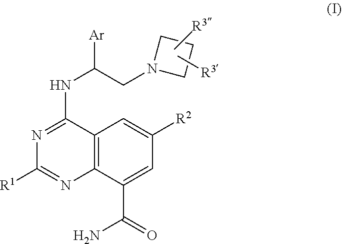

and/or any pharmaceutically acceptable polymorph, enantiomer, stereoisomer, salt, solvate or tautomer thereof, including any mixture thereof in all ratios, wherein:
R¹ is H or LA;
R² is Hal, O(LA), N(LA)(LA)′, CONH(LA), Ar, CONH₂ or A;
R^{3′}, R^{3″} independently are H, LA or Hal;
Ar is a mono- or bicyclic aromatic homo- or heterocycle having 0, 1, 2, 3 or 4 N, O and/or S atoms and 5, 6, 7, 8, 9, or 10 skeleton atoms, which may be unsubstituted or, independently of one another, mono-, di- or trisubstituted by Hal, A, Art, OH, SH, OA, O(Ar1), NH₂, NHA, NH(Ar1), NA₂>NO₂, CN, OCN, SCN, COOH, COOA, CONH₂, CONHA, CONH(Art), CONA₂, NHCOA, NHCO(Art), NHCONHA, NHCONH(Art), NHCONH₂, NHSO₂A, NHSO₂(Ar1), COA, CO(Ar1), SO₂NH₂, SO₂A, SO₂(Ar1) and/or SO₂Hal, and in which a ring N-atom may be substituted by an O-atom to form an N-oxide group, and in which in the case of a bicyclic aromatic cycle on one of the two rings may be partly saturated; Ar is a monocyclic aromatic homo- or heterocycle having 0, 1, 2 or 3 N, O and/or S atoms and 5 or 6 skeleton atoms, which may be unsubstituted or, independently of one another, mono-, di- or trisubstituted by Hal, LA, OH, SH, O(LA), NH₂, NH(LA), N(LA)₂, NO₂, CN, OCN, SCN, COOH, COO(LA), CONH₂, CONH(LA), CON(LA)₂, NHCO(LA), CHO, CO(LA), SO₂NH₂, SO₂(LA) and/or SO₂Hal;
A is unbranched or branched linear or cyclic alkyl having 1, 2, 3, 4, 5, 6, 7 or 8 C atoms, in which one or two CH₂ groups may be replaced by an O or S atom and/or by an —NH—, —CO—, —NHCOO—, —NHCONH—, —N(LA)-, —CONH—, —NHCO— or —CH═CH— group, and in which 1-3H atoms may be replaced by Hal, and in which one or two CH₃ groups may be replaced by OH, SH, NH₂, NH(LA), N(LA)₂, NHCOOH, NHCONH₂ or CN;
LA is unbranched or branched, linear alkyl having 1, 2, 3 or 4 C atoms, wherein 1, 2 or 3H atoms may be replaced by Hal, e.g., methyl, ethyl, trifluoromethyl, difluoromethyl, 1,1,1-trifluoroethyl, propyl, isopropyl, butyl, isobutyl, sec-butyl or tert-butyl; and
Hal is F, Cl or Br, preferably F or Cl, most preferably F, and
(b) an anti-cancer agent, preferably an antineoplastic agent and/or a signal transduction inhibitor.

A preferred composition comprises the combination of (a) and (b) and a pharmaceutically acceptable carrier.

Inventors also describe a combination or composition as described herein for use as a medicament.

In a preferred aspect, the quinazoline carboxamide azetidine compound and the distinct therapeutic agent, preferably the anti-cancer agent, even more preferably the antineoplastic agent and/or the signal transduction inhibitor, of the combination of (a) and (b), are formulated for simultaneous, concurrent or sequential administration, preferably for oral administration.

Preferably, the combination or composition is for use for preventing or treating a hormone dependent disease, preferably cancer, even more preferably for preventing or treating estrogen receptor-positive (ER⁺) cancer in particular breast cancer, in a subject in need thereof, or any mixture thereof in all ratios.

In a preferred aspect, the treatment includes a step involving the administration of an anti-cancer agent, preferably an antineoplastic agent and/or a signal transduction inhibitor, even more preferably an endocrine therapeutic agent for example selected from a Selective ER modulator (“SERM”), a Selective ER down-regulator/degrader (“SERD”), an aromatase Inhibitor (“AI”), a Complete Estrogen Receptor Antagonist (CERAN), an inhibitor of the cell cycle, a PI3K/Akt/mTOR (“PAM”) pathway inhibitor, and/or an inhibitor of the growth factor receptor; more preferably selected from a SERD, a Selective ER modulator (“SERM”), an aromatase Inhibitor (“AI”), a Complete Estrogen Receptor Antagonist (CERAN), a cyclin-dependent kinase (CDK) inhibitor, and any combination thereof; most preferably selected from a Selective ER modulator (“SERM”), a Selective ER down-regulator/degrader (“SERD”), an aromatase Inhibitor (“AI”), a Complete Estrogen Receptor Antagonist (CERAN), and a cyclin-dependent kinase (CDK) inhibitor, a PI3K/Akt/mTOR (“PAM”) pathway inhibitor, and any combination thereof; in addition to the administration of a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, or any mixture thereof in all ratios.

In another particular aspect, the composition comprises (c) an additional agent, preferably an anti-cancer agent. This additional agent is preferably selected from an anti-angiogenic agent, a signal transduction inhibitor, an antineoplastic agent, a therapeutic antibody or a fragment thereof, an antibody-drug conjugate and/or an antisense molecule (in addition to the quinazoline carboxamide azetidine compound, to the antineoplastic agent and/or to the signal transduction inhibitor). This additional agent (c) is preferably selected from an anti-angiogenic agent, a signal transduction inhibitor, an antineoplastic agent, a therapeutic antibody or a fragment thereof, an antibody-drug conjugate, a small molecule, a growth factor receptor agent and/or an antisense molecule (in addition to the quinazoline carboxamide azetidine compound, to the antineoplastic agent and/or to the signal transduction inhibitor).

Also herein described are corresponding treatment methods comprising a step of administering a quinazoline carboxamide azetidine compound, or a combination or (pharmaceutical) composition comprising a quinazoline carboxamide azetidine compound and a pharmaceutically acceptable carrier, preferably (a) a quinazoline carboxamide azetidine compound and (b) a distinct therapeutic agent, preferably an anti-cancer agent, even more preferably an antineoplastic agent and/or a signal transduction inhibitor, and possibly also (c) an additional distinct therapeutic agent, preferably an additional anti-cancer agent, to a subject in need thereof, as further identified herein below. The additional distinct therapeutic agent, preferably anti-cancer agent (c) may be selected for example from an anti-angiogenic agent, a signal transduction inhibitor, an antineoplastic agent, a therapeutic antibody or a fragment thereof, an antibody-drug conjugate and/or an antisense molecule. In a particular aspect, the additional distinct therapeutic agent, preferably anti-cancer agent (c) may be selected for example from an anti-angiogenic agent, a signal transduction inhibitor, an antineoplastic agent, a therapeutic antibody or a fragment thereof, an antibody-drug conjugate, a small molecule, a growth factor receptor agent and/or an antisense molecule.

### Combination of M2698 and of a SERD:

In a more preferred aspect, the treatment includes a step involving the administration of an anti-cancer agent, preferably a Selective ER down-regulator/degrader (“SERD”), preferably selected from amcenestrant, azenosertib (ZN-c5), borestrant, brilanestrant, camizestrant, elacestrant, fulvestrant, giredestrant, imlunestrant, rintodestrant AZD9496, D-0502, LY3484356, GDC-0927 and SHR9549, more preferably elacestrant, in addition to the administration of a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, or any mixture thereof in all ratios.

In a particular aspect, inventors herein describe a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a Selective ER down-regulator Degrader (SERD), preferably a SERD selected from amcenestrant, azenosertib (ZN-c5), borestrant, brilanestrant, camizestrant (AZD9833), elacestrant, fulvestrant, giredestrant, imlunestrant, rintodestrant, AZD9496 (LSZ102), D-0502, LY3484356, GDC-0927, SHR9549, and a pharmaceutically acceptable polymorph, enantiomer, stereoisomer, salt, solvate or tautomer of any of the foregoing, including any mixture thereof in all ratios, even more preferably elacestrant, and a composition comprising the combination of (a) and (b) and a pharmaceutically acceptable carrier.

In a particular aspect, M2698 and/or a pharmaceutically acceptable polymorph, enantiomer, stereoisomer, salt, solvate or tautomer thereof is present in the combination or composition at a dose of about 50 mg to about 800 mg, preferably about 80 mg to about 300 mg and more preferably about 240 mg.

In another particular aspect, M2698 and/or a pharmaceutically acceptable polymorph, enantiomer, stereoisomer, salt, solvate or tautomer thereof is present in the combination or composition at a dose of about 50 mg to about 800 mg, preferably about 80 mg to about 300 mg and more preferably about 240 mg, and elacestrant is present in the combination or composition at a dose of about 150 mg or 200 mg to about 500 mg, for example 172 mg, 258 mg or 345 mg, preferably about 300 mg to about 400 mg, more preferably about 340 mg or 350 mg.

In another particular aspect, M2698 and the SERD are formulated for simultaneous, concurrent or sequential administration, preferably for oral administration.

In another particular aspect, the combination or composition comprises in addition (c) one or several distinct therapeutic compounds, in particular an anti-cancer agent selected from an anti-angiogenic agent, a signal transduction inhibitor, an antineoplastic agent, a therapeutic antibody or a fragment thereof, an antibody-drug conjugate, a small molecule, a growth factor receptor agent, an antisense molecule, and any combination thereof.

Also herein described is a method of treating a hormone dependent disease in a subject, preferably a cancer, wherein the method comprises administering a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a Selective ER down-regulator Degrader (SERD), preferably elacestrant, or a composition comprising the combination of (a) and (b) and a pharmaceutically acceptable carrier, to the subject, thereby treating the subject.

If the hormone dependent disease is a cancer, the cancer may be selected from brain cancer, breast cancer, lung cancer, ovarian cancer, peritoneal cancer, fallopian tube cancer, endometrial cancer, uterine cancer, bladder cancer, colon cancer, prostate cancer, esophageal cancer, liver cancer, pancreatic cancer and stomach cancer. The cancer is preferably a breast cancer, for example selected from a primary cancer, an advanced or metastatic cancer, a human epidermal growth factor receptor 2 positive (HER2+) cancer, a human epidermal growth factor receptor 2 negative (HER2−) cancer, a human epidermal growth factor receptor 2 low (HER2 low) cancer, and a cancer characterized by a wild-type Estrogen Receptor alpha (ERα, ERa or ESR1) cancerous tumor. In a particular aspect, the breast cancer is a cancer characterized by a mutated Estrogen Receptor alpha (ERα, ERa or ESR1) cancerous tumor.

The method of treating a hormone dependent disease in a subject may include the administration of a combination or composition of the invention in combination with (c) one or several distinct therapeutic compounds as herein described. If the hormone dependent disease is a cancer, the one or several distinct therapeutic compounds may be anti-cancer agents for example selected from an anti-angiogenic agent, a signal transduction inhibitor, an antineoplastic agent, a therapeutic antibody or a fragment thereof, an antibody-drug conjugate, a small molecule, a growth factor receptor agent, an antisense molecule, and any combination thereof.

Further herein described is a kit comprising (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a Selective ER down-regulator Degrader (SERD), preferably elacestrant, in different containers, or a composition comprising the combination of (a) and (b) and a pharmaceutically acceptable carrier, and material(s) for administering M2698 and/or the SERD or for administering the composition.

### Combination of M2698 and a Cyclin-Dependent Kinase (CDK) 4/6 Inhibitor:

In another more preferred aspect, the treatment includes a step involving the administration of an anti-cancer agent, preferably a cyclin-dependent kinase (CDK) inhibitor, preferably a CDK4/6 inhibitor, more preferably a CDK4/6 selected from abemaciclib, palbociclib or ribociclib, in addition to the administration of a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof.

In another particular aspect, inventors herein describe a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a cyclin-dependent kinase (CDK) 4/6 inhibitor, preferably selected from abemaciclib, palbociclib, ribociclib and a pharmaceutically acceptable polymorph, enantiomer, stereoisomer, salt, solvate or tautomer of any of the foregoing, including any mixture thereof in all ratios, even more preferably abemaciclib or a pharmaceutically acceptable polymorph, enantiomer, stereoisomer, salt, solvate or tautomer thereof, or a composition comprising the combination of (a) and (b) and a pharmaceutically acceptable carrier.

In a particular aspect, M2698 and/or a pharmaceutically acceptable polymorph, enantiomer, stereoisomer, salt, solvate or tautomer thereof is present in the combination or composition at a dose of about 50 mg to about 800 mg, preferably about 80 mg to about 300 mg and more preferably about 240 mg.

In another particular aspect, M2698 and/or a pharmaceutically acceptable polymorph, enantiomer, stereoisomer, salt, solvate or tautomer thereof is present in the combination or composition at a dose of about 50 mg to about 800 mg, preferably about 80 mg to about 300 mg and more preferably about 240 mg, and wherein abemaciclib is present in the combination or composition at a dose of about 100 mg to about 500 mg, preferably about 150 mg to about 400 mg, more preferably about 300 mg.

In another particular aspect, the M2698 and the CDK4/6 inhibitor are formulated for simultaneous, concurrent or sequential administration, preferably for oral administration.

In another particular aspect, the combination or composition comprises in addition (c) one or several distinct therapeutic compounds, in particular an anti-cancer agent selected from an anti-angiogenic agent, a signal transduction inhibitor, an antineoplastic agent, a therapeutic antibody or a fragment thereof, an antibody-drug conjugate, a small molecule, a growth factor receptor agent, an antisense molecule, and any combination thereof.

If the hormone dependent disease is a cancer, the cancer may be selected from brain cancer, breast cancer, lung cancer, ovarian cancer, peritoneal cancer, fallopian tube cancer, endometrial cancer, uterine cancer, bladder cancer, colon cancer, prostate cancer, esophageal cancer, liver cancer, pancreatic cancer and stomach cancer. The cancer is preferably a breast cancer, for example selected from a primary cancer, an advanced or metastatic cancer, a human epidermal growth factor receptor 2 positive (HER2+) cancer, a human epidermal growth factor receptor 2 negative (HER2−) cancer, a human epidermal growth factor receptor 2 low (HER2 low) cancer, and a cancer characterized by a wild-type Estrogen Receptor alpha (ERα, ERa or ESR1) cancerous tumor. In a particular aspect, the breast cancer is a cancer characterized by a mutated Estrogen Receptor alpha (ERα, ERa or ESR1) cancerous tumor.

Also herein described is a method of treating a hormone dependent disease in a subject, preferably cancer, wherein the method comprises administering a combination of (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a cyclin-dependent kinase (CDK) 4/6 inhibitor, preferably selected from abemaciclib, palbociclib, ribociclib and a pharmaceutically acceptable polymorph, enantiomer, stereoisomer, salt, solvate or tautomer of any of the foregoing, even more preferably abemaciclib or a pharmaceutically acceptable polymorph, enantiomer, stereoisomer, salt, solvate or tautomer thereof, or a composition comprising the combination of (a) and (b) and a pharmaceutically acceptable carrier, to the subject, thereby treating the subject.

Further herein described is a kit comprising (a) 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a cyclin-dependent kinase (CDK) 4/6 inhibitor in different containers, preferably an inhibitor selected from abemaciclib, palbociclib, ribociclib and a pharmaceutically acceptable polymorph, enantiomer, stereoisomer, salt, solvate or tautomer of any of the foregoing, even more preferably abemaciclib or a pharmaceutically acceptable polymorph, enantiomer, stereoisomer, salt, solvate or tautomer thereof, or a composition comprising the combination of (a) and (b) and a pharmaceutically acceptable carrier, and material(s) for administering M2698 and/or the CDK 4/6 inhibitor or for administering the composition.

### Definitions

Unless defined otherwise, all technical and scientific terms used herein have the same meaning as commonly understood by one of ordinary skill in the art to which the subject-matter disclosed herein belongs.

The following definitions may be useful to understand embodiments as presented herein.

The terms “hormone dependent disease” refer to any disease directly or indirectly caused by a hormonal disorder which is the result of hormonal imbalance affecting a gland or organ that produces hormones such as for example the brain, breast, endometrium, ovary, pancreas, prostate, testis, thyroid and bone tissue.

The hormone dependent disease may be for example a cancer, primary amenorrhea, polycystic ovary syndrome (PCOS) or anovulation.

Unless otherwise indicated, the terms “cancer”, “cancerous tumor”, “malignant tumor”, “tumor”, “neoplasia”, “cancer disease”, or “proliferative disease”, are herein used interchangeably. These terms refer to or describe the physiological condition in mammals that is typically characterized by unregulated cell growth. As used herein “cancer” refers to any malignant and/or invasive growth or tumor caused by abnormal cell growth. As used herein “cancer” refers to solid tumors named for the type of cells that form them, as well as cancer of blood, bone marrow, or the lymphatic system. Examples of solid tumors include but are not limited to sarcomas and carcinomas. Examples of cancers of the blood include but are not limited to leukemias, lymphomas and myeloma. The term “cancer” includes but is not limited to a primary cancer that originates at a specific site in the body. The term cancer also includes a cancer that has metastasized, i.e., that has spread from the place in which it started to other parts of the body, for example to the central nervous system (CNS) in particular to the brain, or to the bone, lung, or liver; a recurrence from the original primary cancer after remission, and a second primary cancer that is a new primary cancer in a person with a history of previous cancer of a different type from latter one.

In the context of the invention, a “tumor cell” is a cell obtained from a tumor or tissue of a subject suffering from a cancer, in particular from at least one of the herein identified cancers, preferably breast cancer, and exhibiting well-known hallmarks of cancer cells, e.g. sustaining proliferative signaling, evading growth suppressors, resisting cell death, enabling replicative immortality, inducing angiogenesis, and activating invasion and metastasis. It is to be understood that the expression “tumor cells” used to identify cells obtained from a tumor of a subject, is also used, in the present description, to identify circulating tumor cells, cells obtained from a liquid tumor biopsy, cells obtained from a tumor bed, or cells obtained from a metastasis.

The cancer is preferably a hormone receptor positive (HR+) cancer. The cancer may be selected for example from brain cancer, breast cancer, lung cancer, ovarian cancer, peritoneal cancer, fallopian tube cancer, endometrial cancer, uterine cancer, bladder cancer, colon cancer, prostate cancer, esophageal cancer, liver cancer, pancreatic cancer and stomach cancer.

In some embodiments, the cancer, for example the breast cancer, is hormone receptor positive (HR+), i.e., the cancer is estrogen receptor positive (ER+) and/or progesterone receptor positive (PR+). In some embodiments, the cancer is hormone receptor negative (HR−), i.e., the cancer is estrogen receptor negative (ER−) and progesterone receptor negative (PR−).

The estrogen receptor alpha, also herein identified as “ERα” or “ERa” or “ESR1”, also known as NR3A1 (nuclear receptor subfamily 3, group A, member 1), is a nuclear receptor that is activated by the sex hormone estrogen. It is more particularly one of the two main estrogen receptors subtypes (alpha, ERα, and beta, ERβ) constituting the estrogen receptor (ER). Breast cancers show abundant levels of Era and ERP whereas advanced stages of this disease present only ERα expression, ERβ having been lost. The human wild-type ERa protein amino acid sequence is herein identified as SEQ ID NO: 1. Human ERα is encoded by the gene ESR1 (EStrogen Receptor 1) of SEQ ID NO:3. Alternative splicing results in several ESR1 mRNA transcripts, which differ primarily in their 5-prime unstranslated regions. The translated receptors show less variability.

The estrogen receptor (ER) is a ligand-activated transcription factor composed of several domains important for hormone binding, DNA binding and activation of transcription.

As used herein, a “mutant ERa protein” is a non-wild type ERa protein that contains at least one amino acid mutation (including for example an addition, a deletion, a substitution or a frame shift mutation) relative to the wild type (the protein may also be referred to as “ESR1 WT”).

The “wild type” or “WT” ESR1 or ESR1 refers to the predominant form in which respectively a nucleotide or amino acid sequence exists. The predominant form can be identified in a sample from a subject and/or determined based on the predominant form of the nucleotide or amino acid sequence observed in a subject population, e.g., in the human population. For example, if 80% of nucleotide sequences in the human population contain an adenosine base in a particular location, with the remainder of sequences comprising cytosine, thymine or guanine in that position, wild type is said to have an adenosine in that position. Likewise, if 80% of protein sequences in the human population have a glycine residue in a particular location, with the remainder of sequences comprising some other amino acid residue, glycine is said to be the wild type residue.

As used herein, an “ESR1 mutation” or “ESR1 mutant” or “mutant ERa protein” refers to a non-wild type ERa protein that contains at least one amino acid mutation relative to wild type (the mutant protein may also be referred to as mutant “Era”). As used herein, an “ESR1 mutant” refers to at least one mutation in an ESR1 gene encoding an ERa protein. In some embodiments, the wild type ESR1 gene is SEQ ID NO: 3 and the amino acid sequence of the wild type ERa protein is SEQ ID NO: 1. The skilled artisan will recognize that different ESR1 mutations can result in different ERa proteins with various mutations including for example one or more of the following amino acid sequence mutations: E380Q,V392I, F404fs, V422del, S463P L536H, L536P, L536Q, L536R, Y537C, Y537D, Y537S, Y537N and D538G.

As used herein, a “constitutively active mutant” is a non-wild type protein that is active without the need of a bound ligand, e.g., an ERa protein active even in the absence of estrogen.

The cancer is preferably a cancer conventionally treated with a hormonal (/endocrine) therapy, i.e. a hormone receptor-positive (HR⁺) cancer (cancerous tumor), preferably an estrogen receptor-positive (ER+) cancer. In a preferred embodiment the ER+cancer is selected from breast cancer, ovarian cancer, and type I endometrial cancer. Even more preferably the cancer is a breast cancer, in particular a metastatic and/or advanced breast cancer.

In a particular aspect, the cancer, typically the HR+, preferably ER+, cancer, is a human epidermal growth factor receptor 2 positive (HER2+) cancer.

In another particular aspect, the cancer, typically the HR+, preferably ER+, cancer, is a human epidermal growth factor receptor 2 negative (HER2− or HER2neg) cancer.

In another particular aspect, the cancer, typically the HR+, preferably ER+, cancer, is a human epidermal growth factor receptor 2 low (HER2 low) cancer.

In a particular aspect, the tumor is a HR+, in particular ER+, and “mutated ESR1” or “mutated ERa” tumor.

This tumor may also be a HR+, in particular ER+, mutated ESR1 (or mutated ERa) and HER2-/HER2neg tumor. In some such aspects, the HR+/HER2− cancer, in particular breast cancer, is refractory to conventional treatment of cancer treatment hereafter defined and preferably with an antineoplastic agent preferably an endocrine therapeutic agent selected for example from a Selective ER Modulator (SERM), a Selective ER down-regulator Degrader (SERD), an Aromatase Inhibitor (AI) inhibitor, a CERAN, an hormone in particular a Luteinizing hormone-releasing hormone (LHRH) agonist, a gonadotropin releasing hormone (GnRH) agonist, a cyclin-dependent kinase (CDK) inhibitor, for example a CDK4/6 inhibitor (such as palbociclib, ribociclib or abemaciclib), and a pharmaceutically acceptable salt thereof. In some such embodiments, the HR+/HER2− breast cancer is resistant to treatment with an endocrine therapeutic agent selected for example from a Selective ER Modulator (SERM), a Selective ER down-regulator Degrader (SERD), an Aromatase Inhibitor (AI) inhibitor, a CERAN, an hormone in particular a Luteinizing hormone-releasing hormone (LHRH) agonist, a gonadotropin releasing hormone (GnRH) agonist a cyclin-dependent kinase (CDK) inhibitor preferably a CDK4/6 inhibitor (such as palbociclib, ribociclib or abemaciclib), and a pharmaceutically acceptable salt thereof. In some embodiments, the HR+/HER2− breast cancer is characterized by amplification or overexpression of cyclin E1 (CCNE1) and/or cyclin E2 (CCNE2).

In some other aspects, the HR+/HER2− cancer, in particular breast cancer, is characterized by amplification or overexpression of cyclin E1 (CCNE1). In some aspects of each of the foregoing, the HR+/HER2− cancer is advanced or metastatic HR+/HER2− breast cancer.

In some other aspects, the cancer is HR−/HER2+, in particular HR−/HER2+ breast cancer. In some embodiments wherein the cancer is HER2+, the methods, combinations and uses described herein further comprise an additional anti-cancer agent, wherein the additional anti-cancer agent is a HER2-targeted agent (e.g., trastuzumab emtansine, fam-trastuzumab deruxtecan, pertuzumab, lapatinib, neratinib or tucatinib), or an agent targeting the PI3K/AKT molecular pathway (e.g., ipatasertib).

In another particular aspect, the tumor is a HR+, in particular ER+, mutated ESR1 (or mutated ERa) and HER2+/HER2pos tumor.

In another particular aspect, the tumor is a HR+, in particular ER+, mutated ESR1 (or mutated ERa) and HER2 low tumor.

In some aspects, the breast cancer is associated with the BRCA1 or BRCA2 gene.

In another aspect, the breast cancer is triple negative breast cancer (TNBC), i.e., the breast cancer is ER−, PR− and HER2−. In some aspects, the TNBC is refractory to treatment with an endocrine therapeutic agent selected for example from a Selective ER Modulator (SERM), a Selective ER down-regulator Degrader (SERD), an Aromatase Inhibitor (AI) inhibitor, a CERAN, an hormone in particular a Luteinizing hormone-releasing hormone (LHRH) agonist, a gonadotropin releasing hormone (GnRH) agonist, a cyclin-dependent kinase (CDK) inhibitor preferably a CDK4/6 inhibitor (such as palbociclib, ribociclib or abemaciclib), and a pharmaceutically acceptable salt thereof. In some aspects, the TNBC is resistant to treatment with an endocrine therapeutic agent selected for example from a Selective ER Modulator (SERM), a Selective ER down-regulator Degrader (SERD), an Aromatase Inhibitor (AI) inhibitor, a CERAN, an hormone in particular a Luteinizing hormone-releasing hormone (LHRH) agonist, a gonadotropin releasing hormone (GnRH) agonist, a cyclin-dependent kinase (CDK) inhibitor preferably a CDK4/6 inhibitor (such as palbociclib, ribociclib or abemaciclib), and a pharmaceutically acceptable salt thereof. In some aspects, the TNBC is characterized by amplification or overexpression of cyclin E1 (CCNE1) and/or cyclin E2 (CCNE2). In some such aspects, the TNBC is characterized by amplification or overexpression of cyclin E1 (CCNE1). In some aspects, the TNBC is locally recurrent/advanced or metastatic TNBC. In some aspects, the TNBC is advanced or metastatic TNBC. In a particular embodiment, the breast cancer is an advanced or metastatic breast cancer characterized by a mutated Estrogen Receptor alpha (ERα, ERa or ESR1) cancerous tumor wherein the mutation occurs in the ligand-binding domain of the ERa wild-type sequence of SEQ ID NO: 1, and/or wherein the mutated ERa is characterized by a modification of the conformation of its ligand-binding domain.

In a preferred embodiment, the mutation occurs on at least one residue selected from residue 380, 392, 404, 422, 463, 536, 537 and 538, and is even more preferably an amino acid substitution selected from E380Q,V392I, F404fs, V422del, S463P, L536H, L536P, L536Q, L536R, Y537C, Y537D, Y537S, Y537N, D538G, and is even more preferably Y537S and/or D538G.

In some aspects of each of the foregoing, the cancer, in particular the breast cancer, is refractory or resistant to treatment with one or more standard of care agents.

In some such embodiments, the breast cancer is refractory or resistant to treatment with an antineoplastic agent preferably an endocrine/hormonal therapeutic agent, such as for example AIs, CERAN, SERD, or SERM. In some embodiments, the cancer, in particular the breast cancer, is refractory or resistant to treatment with a cyclin-dependent kinase (CDK) inhibitor preferably a CDK4/6 inhibitor. For example, in some embodiments, the breast cancer is refractory or resistant to treatment with palbociclib, ribociclib or abemaciclib, or any pharmaceutically acceptable polymorph, enantiomer, stereoisomer, salt, solvate or tautomer thereof. In other embodiments, the breast cancer is refractory or resistant to treatment with, or has progressed on, treatment with antineoplastic chemotherapeutic agents such as platinum agents, taxanes, anthracyclines or anti-metabolites.

In the context of the present invention, a “conventional treatment of cancer” (also herein identified as “standard-of-care treatment” or “main mode of cancer therapy”) may be an anti-cancer agent selected for example from an endocrine (/hormonal) therapeutic agent, an anti-angiogenic agent, a signal transduction inhibitor, an antineoplastic agent, a small molecule, an antibody or a fragment thereof (including a single chain antibody), an antibody-drug conjugate (ADC), and an antisense molecule, preferably from an anti-angiogenic agent, a signal transduction inhibitor, an antineoplastic agent, a small molecule, a growth factor receptor agent, an antibody or a fragment thereof (including a single chain antibody), an antibody-drug conjugate (ADC), and an antisense molecule.

The conventional treatment of cancer is for example selected from a hormonotherapy, an immunotherapy, a specific kinase inhibitor-based therapy, an antiangiogenic agent based-therapy, an antibody-based therapy, in particular a monoclonal antibody-based therapy, in particular an antibody drug conjugate-based therapy, a chemotherapy, a radiation therapy and surgery.

The term “conventionally” means that the therapy is applied or, if not routinely applied, is appropriate and at least recommended by health authorities. The “conventional” treatment is chosen by the oncologist depending on the specific cancer to be prevented or treated. The term “adjuvant therapy” refers to additional treatment given after a main mode of therapy.

In the context of cancer, a conventional treatment of cancer is chemotherapy. The chemotherapeutic agent may be selected for example from an alkylating agent, a platinum coordination complex, a cytotoxic antibiotic, an antimetabolite, a taxane, a topoisomerase inhibitor and a vinca alkaloid.

In the context of cancer, in particular a hormone receptor-positive (HR+) cancer such as breast cancer, a conventional treatment of cancer is an hormonal treatment which involves the administration of a drug selected from a Selective ER Modulator (SERM), a Selective ER down-regulator Degrader (SERD), an Aromatase Inhibitor (AI) and a Complete Estrogen Receptor Antagonist (CERAN). In a particular aspect, the conventional treatment of such a HR+ cancer involves the use of an antineoplastic agent and/or of a signal transduction inhibitor.

The conventional treatment of cancer preferably involves a Cyclin-dependent kinase (CDK) inhibitor, an endocrine therapeutic agent selected for example from a Selective ER Modulator (SERM), a Selective ER down-regulator Degrader (SERD), an Aromatase Inhibitor (AI) inhibitor, a CERAN, an hormone in particular a Luteinizing hormone-releasing hormone (LHRH) agonist, a gonadotropin releasing hormone (GnRH) agonist, a progestin or an anti-androgen, a CYP17 inhibitor, an adrenolytic agent; a PI3K/AKT/mTOR (“PAM”) pathway inhibitor, and any combination thereof, in particular the combination of a CDK inhibitor and a hormonal therapeutic agent, a CDK inhibitor and a SERD and/or a SERM, or the combination of a CDK inhibitor, a SERD and a PI3K/AKT/mTOR (“PAM”) pathway inhibitor.

Particular conventional treatments of cancer involve the administration of a drug selected from an inhibitor of a compound of the ER pathway, of the PI3K/Akt/mTOR (“PAM”) pathway, of a growth factor receptor pathway(s) [involving the epidermal growth factor receptor (EGFR), fibroblast growth factor receptor (FGFR), insulin growth factor receptor (IGFR) and/or vascular endothelial growth factor receptor (VEGFR)], of the cell cycle, of the ubiquitin-proteasome pathway, or of the bromodomain and/or extra-terminal domain of proteins. Preferably said drug is an inhibitor of the cell cycle or an inhibitor of the PI3K/AKT/mTOR (“PAM”) pathway, or a targeted agent, typically a anti-hormone-receptor targeted agent such as a HER-2 targeted agent.

Other conventional treatments, in particular adjuvant treatments, of cancer include surgery, radiation therapy (also herein identified as “radiotherapy”), chemotherapy, immunotherapy, endocrine therapy and targeted therapy (in particular a targeted therapy focusing on a tumor's specific gene or protein).

In a particular aspect, the conventional treatment is not an EGFR inhibitor, in particular the treatment is not an EGFR inhibitor selected from gefitinib, erlotinib, afatinib, brigatinib, icotinib, osimertinib and cetuximab. In a particular aspect, the treatment is not cetuximab.

The term “anti-angiogenic agent” designates in particular a VEGF inhibitor, VEGFR inhibitor, TIE-2 inhibitor, PDGFR inhibitor, angiopoietin inhibitor, PKCf3 inhibitor, cyclooxygenase II (COX-2) inhibitor, integrin (alpha-v/beta-3), matrix-metalloproteinase 2 (MMP-2) inhibitor or matrix-metalloproteinase 9 (MMP-9) inhibitor. The anti-angiogenic agent may be selected for example from gefitinib, Cilengitide, Ziv-aflibercept (Zaltrap®) and a pharmaceutically acceptable polymorph, enantiomer, stereoisomer, salt, solvate or tautomer form of any of the foregoing. It is preferably selected from cilengitide, Ziv-aflibercept (Zaltrap®) and a pharmaceutically acceptable polymorph, enantiomer, stereoisomer, salt, solvate or tautomer form of any of the foregoing.

The term “signal transduction inhibitor” designates in particular a kinase inhibitor [e.g., an inhibitor of a tyrosine kinase, serine/threonine kinase including for example a p21-activated kinase 1 (PAKI), cyclin dependent kinase, or a member of the HER family of tyrosine kinases such as Epidermal Growth Factor Receptor-2 (HER2) or Receptor-3 (HER3)], a proteasome inhibitor, an Isocitrate dehydrogenase 1 and 2 (IDH1 and IDH2) inhibitor, a neurotrophin receptor kinase (NTRK) inhibitor, a Hedgehog pathway inhibitor, a PARP inhibitor, a B-cell lymphoma 2 (BCL2) inhibitor, a EZH2 Inhibitor, a FGFR pathway inhibitor, a Rearranged during Transfection (RET) inhibitor, a Notch inhibitor, a selective inhibitor of nuclear export (SINE), a SRC3 inhibitor, a MYC inhibitor, a BET bromodomain inhibitor, a Farnesyl transferase inhibitor, a HMG-CoA reductase inhibitor, a PI3K/AKT/mTOR (“PAM”) pathway inhibitor, a RAS/RAF/MEK/ERK (“MAPK”) pathway inhibitor, a S6K inhibitor, a elF4A/E inhibitor or a CDK inhibitor.

The kinase inhibitor may be selected for example from abivertinib, acalabrutinib, afatinib, alectinib, almonertinib, azenosertib (ZN-c5), axitinib, binimetinib (Mektovi or ARRY-162), bosutinib, brigatinib, ceritinib, cobimetinib, crizotinib, dabrafenib, dacomitinib, dasatinib, encorafenib, epertinib, erlotinib, gefitinib, gilteritinib, ibrutinib, icotinib, imatinib, lapatinib, larotinib, larotrectinib, lazertinib, lifirafenib, lorlatinib, midostaurin, mobocertinib, naquotinib, nazartinib, neratinib, nilotinib, olmutinib, osimertinib, pazopanib, pimasertib, ponatinib, poziotinib, pyrotinib, refametinib, regorafenib, ruxolitinib, sapitinib, selatinib, sirotinib, tepotinib, tesevatinib, trametinib, tucatinib, varlitinib, vemurafenib, yinlitinib, zanubrutinib, zorifertinib, ABP-1119, ABP-1130, AG-101, AI-6802, AM-105, AMX-3009, APL-1898, ASK-120067, AST-2818, BAY-2476568, BBT-176, BDTX-189, BEBT-108, BEBT-109, BH-2922, BI-4020, BLU-4810, BMX-002, BO-1978, BPI-15086, BPI-7711 C-005, DS-2087b, CK-101, CLM-29, CLM-3, CMAB-017, CR-13626, CSHEGF-29, D-0316, DBPR-112, DGD-1202, DTRMWXHS-12, DZD-9008, EO-1001, ES-072, FCN-411, FHND-9041, FLAG-001, FLAG-003, FmAb-2, GB-263, GC-1118A, HA-12128, HMPL-309, HMPL-813, HS-627, IPA3, JMT-101, JRF-103, JS-111, JZB-29, KBP-5209, KNP-501, KU-004, LL-191, MCLA-129, MCLA-158, MDC-22, MP 0274, mRX-7, MTX-211, MVC-101, NRC-2694, NT-004, NT-113, OBX-1012, ORIC-114, PB-357, PF-0779954, QL-1 105, QL-1203, RXDX-105, SAH-EJ1, SCT-200, SKLB-1028, SKLB-1206, SPH-118811, SYN-004, TAS-6417, TGRX-360, TQB-3804, UBP-1215, VRN-071918, VRN-6, WBP-297, WJ-13404, WSD-0922, XZP-5809, YZJ-0318, ZNE-4, ZR-2002, ZSP-0391, ZW49 and a pharmaceutically acceptable polymorph, enantiomer, stereoisomer, salt, solvate or tautomer of any of the foregoing.

The p21-activated kinase 1 (PAKI) inhibitor may be for example IPA3 or a pharmaceutically acceptable polymorph, enantiomer, stereoisomer, salt, solvate or tautomer thereof.

In a particular embodiment, the signal transduction inhibitor is not a kinase inhibitor.

The proteasome inhibitor may be selected for example from bortezomib, carfilzomib, ixazomib, marizomib, and a pharmaceutically acceptable polymorph, enantiomer, stereoisomer, salt, solvate or tautomer of any of the foregoing.

The isocitrate dehydrogenase 1 and 2 (IDH1 and IDH2) inhibitor may be for example enasidenib, ivosidenib or a pharmaceutically acceptable polymorph, enantiomer, stereoisomer, salt, solvate or tautomer of any of the foregoing.

The neurotrophin receptor kinase (NTRK) inhibitor may be selected for example from entrectinib, sunitinib, M074-2865, PF-07265028 or a pharmaceutically acceptable polymorph, enantiomer, stereoisomer, salt, solvate or tautomer of any of the foregoing.

The Hedgehog pathway inhibitor may be selected for example from glasdegib, sonidegib, vismodegib and a pharmaceutically acceptable polymorph, enantiomer, stereoisomer, salt, solvate or tautomer of any of the foregoing.

The PARP inhibitor may be selected for example from niraparib, olaparib, rucaparib, talazoparib and a pharmaceutically acceptable polymorph, enantiomer, stereoisomer, salt, solvate or tautomer of any of the foregoing.

The B-cell lymphoma 2 (BCL2) inhibitor may be for example venetoclax or a pharmaceutically acceptable polymorph, enantiomer, stereoisomer, salt, solvate or tautomer thereof.

The EZH2 Inhibitor may be for example tazemetostat or PF-06821497, or a pharmaceutically acceptable polymorph, enantiomer, stereoisomer, salt, solvate or tautomer of any of the foregoing.

The FGFR pathway inhibitor may be selected for example from lucitanib, dovitinib, AZD4547, erdafitinib, infigratinib (BGJ398), BAY-1163877 and a pharmaceutically acceptable polymorph, enantiomer, stereoisomer, salt, solvate or tautomer of any of the foregoing.

The Rearranged during Transfection (RET) inhibitor may be selected for example from vandetanib, cabozantinib, lenvatinib, sorafenib, selpercatinib, pralsetinib and a pharmaceutically acceptable polymorph, enantiomer, stereoisomer, salt, solvate or tautomer of any of the foregoing.

The Notch inhibitor may be for example MK-0752, [(s,s)-2-(3,5-difluorophenyl)-acetylamino]-N-(1-methyl-2-oxo-5-phenyl-2,3-dihydro-1H-benzo[e][1,4]diazepin-3-yl)-propionamide, or 11-endo-N-(5,6,7,8,9,10-hexahydro-6,9-methano benzo[9][8]annulen-11-yl)-thiophene-2-sulfonamide or a pharmaceutically acceptable polymorph, enantiomer, stereoisomer, salt, solvate or tautomer of any of the foregoing.

The selective inhibitor of nuclear export (SINE) may be for example selinexor or eltanexor (KPT-8602) or a pharmaceutically acceptable polymorph, enantiomer, stereoisomer, salt, solvate or tautomer of any of the foregoing.

The SRC3 inhibitor may be for example SI-2 or a pharmaceutically acceptable polymorph, enantiomer, stereoisomer, salt, solvate or tautomer thereof.

The MYC inhibitor may be for example omomyc or a pharmaceutically acceptable polymorph, enantiomer, stereoisomer, salt, solvate or tautomer thereof.

The BET bromodomain inhibitor may be selected for example from JQ1, I-BET762, OTX015, I-BET151, RVX-208, MS417, ABBV-075, ABBV-744, SJ432, AZD5153, INCB054329 and a pharmaceutically acceptable polymorph, enantiomer, stereoisomer, salt, solvate or tautomer of any of the foregoing.

The Farnesyl transferase inhibitor may be for example lonafarnib or tipifarnib or a pharmaceutically acceptable polymorph, enantiomer, stereoisomer, salt, solvate or tautomer of any of the foregoing.

The HMG-CoA reductase inhibitor may be for example atorvastatin (Caduet, Lipitor, Lypqozet) or a pharmaceutically acceptable polymorph, enantiomer, stereoisomer, salt, solvate or tautomer thereof.

The PI3K/AKT/mTOR (“PAM”) pathway inhibitor designates a compound that is designed to inhibit the phosphoinositide 3-kinase (PI3K) and/or the Protein kinase B (Akt) and/or the mammalian target of rapamycin (mTOR). It is for example a phosphoinositide 3-kinase (PI3K) pathway inhibitor, an ATP-competitor, a dual inhibitor of class I PI3K and mTORC1/2; a “pan-PI3K” inhibitor which inhibits all four isoforms of class I PI3K (α, β, δ, γ); an isoform-specific inhibitor of anyone of the various PI3K isoforms such as class II PI3K (PI3K-C2α, PI3K-C2β, and PI3K-C2γ) or class III PI3K; an allosteric and catalytic inhibitor of AKT; an ATP-competitive inhibitor of mTOR only (and thus of both mTORC1 and mTORC2) or a pharmaceutically acceptable polymorph, enantiomer, stereoisomer, salt, solvate or tautomer of any of the foregoing. The PAM pathway inhibitor may be selected for example from a mTOR inhibitor such as bimiralisib, dactolisib tosylate, detorsertib, everolimus, monepantel, omipalisib, onatasertib, ridaforolimus, sapanisertib, sirolimus, Streptomyces sp OA293, temsirolimus, vitusertib, AL-5880S, AL-58922, AUM-302, CA-102, CA-103, CC-115, CC-223, CT-365, DFN-S29, DHM-ZS, FP-208, FT-I S18, HEC-68498, LXI-15029, ME-344, NSC-765844, OSI-027, OSU-53, OT-043, PQR-514, PTX-367, QR-213, RMC-5552, SN-202, SPR-965 or TAM-03, WXFL-10030390 or XP-105; a AKT/PDK1 inhibitor such as afuresertib, borussertib, capivasertib (AZD 5363), celecoxib or a celecoxib derivative, dordaviprone (ONC-201), enzastaurin, ipatasertib, miransertib, uprosertib (GSK2141795), ALM-301, ARQ-751, AT-13148, AZD8055, BAY-1125976, BX795, BX912, COTI-2, DC-120, FXY-1, GSK470, JRP-890, JX06, KS-99, LY-2503029, MK-2206, NISC-6, OSU-03012, PHT-427, PTX-200, RX-0201, RX-0301, SBF1 or TAS-117; a PI3K inhibitor such as alpelisib, buparlisib (BKM120), copanlisib, duvelisib, idelalisib, paxalisib, pictilisib (GDC0941), or sonalisib; and a pharmaceutically acceptable polymorph, enantiomer, stereoisomer, salt, solvate or tautomer of any of the foregoing. The PAM inhibitor may also be selected from AZD8055, GDC0941, selumetinib and a pharmaceutically acceptable polymorph, enantiomer, stereoisomer, salt, solvate or tautomer of any of the foregoing. The inhibitor of the PAM pathway is preferably selected from buparlisib, pilaralisib, pictilisib, sonolisib, dactolisib, sapanisertib, voxtalisib, serabelisib, alpelisib, perifosine, MK2206, ipatasertib, GSK690693, temsirolimus, ridaforolimus, sirolimus, everolimus, AZD8055, OSI-027 and a pharmaceutically acceptable polymorph, enantiomer, stereoisomer, salt, solvate or tautomer of any of the foregoing, or any mixture thereof in all ratios.

The RAS/RAF/MEK/ERK (“MAPK”) pathway inhibitor may be selected for example from VS-6766, TNO-155, SHP099, RMC-4550, RMC-4630, RMC-5845, 1ACS-13909, JAB-3068, JAB-3312, RLY-1971, BBP-398, ERAS-601, HBI-2376, ICP-189, BR790, ETS-001, PF-07284892, RX-SHP2i, SH3809, TAS-ASTX, X-37-SHP2, BMS-SCH, BAY-293, BI-3406, BI-1701963, SDGR-5, AZ6197, BIERKi, CC-90003, ERAS-007, HMPL-295, IPN-ERK, KO-947, LTT462, SCH772984, TK216, ASTX-029, HH-2710, LY-3214996, selumetinib, trametinib, ulixertinib, ASN-007, ATG-017, BPI-27336, JSI-1187, MK-8353, JRP-890, JRF-108, a dual RAF/MEK inhibitor as identified in WO2022/170060 and a pharmaceutically acceptable polymorph, enantiomer, stereoisomer, salt, solvate or tautomer of any of the foregoing.

In a particular aspect, the MAPK pathway inhibitor is not a MEK inhibitor.

In a particular aspect, the MEK inhibitor is not trametinib, cobimetinib, selumetinib, refametinib or pimasertib. Preferably, the MEK inhibitor is not pimasertib.

The S6K inhibitor may be selected for example from LY2584702, a piperazinyl-pyrimidine derivative (such as PF-4708671 or PF-4708671), A77 1726 (active metabolite of leflunomide), FS-115, FL772, LY2780301, LYS6K2, AD80/AD81, gingerenone and a pharmaceutically acceptable polymorph, enantiomer, stereoisomer, salt, solvate or tautomer of any of the foregoing.

The elF4A/E inhibitor may be selected for example from a rapamycin analogue, ribavirin, zotatifin, and a pharmaceutically acceptable polymorph, enantiomer, stereoisomer, salt, solvate or tautomer of any of the foregoing.

The Cyclin-dependent kinase (CDK)inhibitor is selected for example from a CDK 1, 2, 4, 5, 6 and/or 7 inhibitor, and may be in particular and for example abemaciclib (also known as LY2835219), AG024322, aloisine A, aloisine B, alsterpaullone, aminopurvalanol, AT7519, AZD-5438, AZD5597, BLU-222, BMS-387032, birociclib (XZP 3287), bohemine, butyrolactone, CYC065, dalpiciclib (SHR-6390), dinaciclib, ETH-155008, flavopiridol, FCN-437c, GLR2007, indirubin, indirubin-3′-monoxime, JNJ-7706621, kenpaullone, lerociclib (also known as G1T38), meriolin 3, milciclib, narazaciclib (ON123300), NVP-LCQ19, olomoucine, olomoucine II, palbociclib (also known as Ibrance, PD-0332991 or PF-00080665), PF-07220060, PF-07104091, PF-06873600, PHA-793887, purvalanol A, purvanol B, R-CR8, RGB-286638, RGB286147, ribociclib (also known as LEE-011), riviciclib hydrochloride (P276-00), roniciclib, R-roscovitine, Ro4584820, SRX-3177, TG02, TQB3303, trilaciclib (also known as GTI128), voruciclib, xylocydin, ZK304709, 10Z-Hymenialdisine, 5-Iodo-indirubin-3′-monoxime, (1R,3S)-3-[3-(([3-(methoxymethyl)-1-methyl-1H-pyrazol-5-yl]carbonyl)amino)-1Hpyrazol-5-yl]cyclopentyl propan-2-ylcarbamate, or a pharmaceutically acceptable polymorph, enantiomer, stereoisomer, salt, solvate or tautomer of any of the foregoing. A particular example of a CDK7 inhibitor is samuraciclib (also known as CT7001 or ICE0942).

Other examples of CDK inhibitors are described in WO2022/018596. The CDK inhibitor is preferably a CDK4/6 inhibitor such as for example abemaciclib, palbocilcib, ribociclib and/or a pharmaceutically acceptable polymorph, enantiomer, stereoisomer, salt, solvate or tautomer of any of the foregoing.

The term “antineoplastic agent” designates in particular a chemotherapeutic agent and a hormonal (/endocrine) therapeutic agent.

The term “chemotherapeutic agent” designates in particular an alkylating agent, a platinum coordination complex, a cytotoxic antibiotic, an antimetabolite, a biologic response modifier, a histone deacetylase (HDAC) inhibitor, a growth factor inhibitor, a taxane, a topoisomerase inhibitor or a vinca alkaloid. The term also covers other compounds such as for example asparaginase (pegaspargase), bexarotene, eribulin, eribulin mesylate fosbretabulin, hydroxyurea, ixabepilone, lenalidomide, mitotane, omacetaxine, pomalidomide, tagraxofusp, telotristat, thalidomide and pharmaceutically acceptable polymorphs, enantiomers, stereoisomers, salts, solvates and tautomers of any of the foregoing.

In a preferred aspect, the chemotherapeutic agent is selected from capecitabin, cyclophosphamide, docetaxel, doxorubicin, epirubicin, eribulin mesylate, fluorouracil, 5-fluorouracil, gemcitabine, liposomal doxorubicin, paclitaxel, vinorelbine, and a pharmaceutically acceptable polymorph, enantiomer, stereoisomer, salt, solvate or tautomer of any of the foregoing.

The alkylating agent may be selected for example from apaziquone, altretamine, bendamustine, busulfan, carboquone, carmustine, chlorambucil, chlormethine, cyclophosphamide, dacarbazine, fotemustine, glufosfamide, ifosfamide, improsulfan tosilate, lomustine, mechlorethamine, melphalan, mitobronitol, mitolactol, nimustine, palifosfamide, pipobroman, procarbazine, ranimustine, streptozocin, temozolomide, N,N′N′-triethylenethiophosphoramide (ThioTEPA), trabectedin, treosulfan. Trofosfamide, uramustine and a pharmaceutically acceptable polymorph, enantiomer, stereoisomer, salt, solvate or tautomer of any of the foregoing.

The platinum coordination complex may be selected for example from carboplatin, cisplatin, eptaplatin, miriplatine hydrate, oxaliplatin, lobaplatin, nedaplatin, picoplatin, satraplatin and a pharmaceutically acceptable polymorph, enantiomer, stereoisomer, salt, solvate or tautomer of any of the foregoing.

The cytotoxic antibiotic may be selected for example from aclarubicin, bleomycin, dactinomycin, daunorubicin, doxorubicin, epirubicin, levamisole, idarubicin, miltefosine, mitomycin, mitoxantrone, plicamycin, valrubicin and a pharmaceutically acceptable polymorph, enantiomer, stereoisomer, salt, solvate or tautomer of any of the foregoing.

The antimetabolite may be selected for example from an antifolate (such as methotrexate, pemetrexed, pralatrexate or trimetrexate); a purine analogue (such as azathioprine, cladribine, fludarabine, mercaptopurine or thioguanine); a pyrimidine analogue (such as azacitidine, capecitabine, cytarabine, decitabine, floxuridine, fluorouracil, 5-fluorouracil, gemcitabine or trifluridine/tipracil) and a pharmaceutically acceptable polymorph, enantiomer, stereoisomer, salt, solvate or tautomer of any of the foregoing.

The biologic response modifier may be selected for example from aldesleukin (IL-2), denileukin diftitox, and interferon gamma.

The histone deacetylase (HDAC) inhibitor may be selected for example from belinostat, Panobinostat, vorinostat, romidepsin and a pharmaceutically acceptable polymorph, enantiomer, stereoisomer, salt, solvate or tautomer of any of the foregoing.

The taxane may be selected for example from cabazitaxel, docetaxel, paclitaxel (for example a paclitaxel albumin-stabilized nanoparticle formulation), tesetaxel and a pharmaceutically acceptable polymorph, enantiomer, stereoisomer, salt, solvate or tautomer of any of the foregoing.

The topoisomerase inhibitor be selected for example from etoposide, irinotecan, teniposide, topotecan and a pharmaceutically acceptable polymorph, enantiomer, stereoisomer, salt, solvate or tautomer of any of the foregoing.

The vinca alkaloid be selected for example from vinblastine, vindesine, vincristine, vinflunine, vinorelbine and a pharmaceutically acceptable polymorph, enantiomer, stereoisomer, salt, solvate or tautomer of any of the foregoing.

The term “hormonal (/endocrine) therapeutic agent” designates in particular an antiandrogen such as abiraterone (abiraterone acetate), apalutamide, bicalutamide, cyproterone, enzalutamide, flutamide, and nilutamide; an hormone such as a Luteinizing Hormone-Releasing Hormone (LHRH) agonist or a Gonadotropin Releasing Hormone (GnRH) agonist such as buserelin, cetrorelix, gonadorelin, lanreotide, octreotide, somatostatine (pasireotide), degarelix, goserelin, leuprolide or triptorelin, or triptorelix; a progestin such as medroxyprogesterone acetate or megestrol acetate; a CYP17 inhibitor such as abiraterone or ketoconazole; an adrenolytic agent such as mitotane; an antiestrogen; and a pharmaceutically acceptable polymorph, enantiomer, stereoisomer, salt, solvate or tautomer of any of the foregoing.

The antiestrogen may be selected for example from a Selective ER down-regulator or Selective ER degraders (“SERD”), a Selective ER Modulator (“SERM”), an aromatase Inhibitor (“AI”) and a Complete Estrogen Receptor Antagonist (“CERAN”).

The term “Selective ER Modulator (“SERM”)” designates anti-estrogens compounds that are designed to compete with estrogen for ER binding and show mixed agonist/antagonist capabilities in a tissue-specific fashion. The SERM is typically selected from a triphenylethylene, a benzothiophene, a phenylindole, a tetrahydronaphthalene, and a pharmaceutically acceptable polymorph, enantiomer, stereoisomer, salt, solvate or tautomer of any of the foregoing.

The triphenylethylene is preferably tamoxifen or a “tamoxifen-like” compound such as 4 hydroxytamoxifen, afimoxifene, endoxifen, toremifene, droloxifene or idoxifene; the benzothiophene is preferably raloxifene or arzoxifene; the phenylindole is preferably bazedoxifene or pipindoxifene; the tetrahydronaphthalene is preferably lasofoxifene.

The terms “Selective ER down-regulator or Selective ER degraders (“SERD”)” designate anti-estrogens compounds designed to create an unstable protein complex, inducing ER protein degradation via proteasome. The SERD is preferably selected from fulvestrant, elacestrant [(R)-6-(2-(ethyl(4-(2-(ethylamino)ethyl)benzyl)amino)-4-methoxyphenyl)-5, 6,7,8-tetrahydronaphthalen-2-ol, also identified as RAD-1901], amcenestrant [(S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid, also identified as SAR439859], brilanestrant [(E)-3-(4-((E)-2-(2-chloro-4-fluorophenyl)-1-(1H-indazol-5-yl)but-1-en-1-yl)phenyl)acrylic acid, also identified as ARN-810 or GDC-0810], camizestrant [N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine, also identified as AZD9833], giredestrant [3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol, also identified as RG6171 or GDC9545], rintodestrant [(E)-3-(4-((2-(4-fluoro-2,6-dimethylbenzoyl)-6-hydroxybenzo[b]thiophen-3-yl)oxy)phenyl)acrylic acid, also identified as G1T48], AZD9496 [(E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid], (E)-3-(4-((2-(2-(1,1-difluoroethyl)-4-fluorophenyl)-6-hydroxybenzo [b]thiophen-3-yl)oxy)phenyl)acrylic acid (also identified as LSZ102), D-0502, LY3484356, GDC-0927, SHR9549, and a pharmaceutically acceptable polymorph, enantiomer, stereoisomer, salt, solvate or tautomer of any of the foregoing. In another preferred aspect, the SERD compound is elacestrant or a pharmaceutically acceptable polymorph, enantiomer, stereoisomer, salt, solvate or tautomer thereof.

In a particularly preferred embodiment, the SERD is selected from amcenestrant [(S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid, also identified as SAR439859], azenosertib (ZN-c5) [1-[(7R)-7-ethyl-7-hydroxy-5,6-dihydrocyclopenta[b]pyridin-2-yl]-6-[4-(4-methylpiperazin-1-yl)anilino]-2-prop-2-enylpyrazolo[3,4-d]pyrimidin-3-one], borestrant [(7a,17b)-7-[9-[(4,4,5,5,5pentafluoropentyl)Sulfinyl]nonyl]estra-1,3,5(10)-triene-3,17-diol-3 boronic acid], brilanestrant [(E)-3-(4-((E)-2-(2-chloro-4-fluorophenyl)-1-(1H-indazol-5-yl)but-1-en-1-yl)phenyl)acrylic acid, also identified as ARN-810 or GDC-0810], camizestrant [N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine, also identified as AZD9833], elacestrant [(R)-6-(2-(ethyl(4-(2-(ethylamino)ethyl)benzyl)amino)-4-methoxyphenyl)-5,6,7,8-tetrahydronaphthalen-2-ol, also identified as RAD-1901], fulvestrant [(7a,17b)-7-[9-[(4,4,5,5,5pentafluoropentyl)Sulfinyl]nonyl]estra-1,3,5(10)-triene-3,17-diol], giredestrant [3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol, also identified as RG6171 or GDC9545], imlunestrant [(5R)-5-[4-[2-[3-(fluoromethyl)azetidin-1-yl]ethoxy]phenyl]-8-(trifluoromethyl)-5H-chromeno[4,3-c]quinolin-2-ol], rintodestrant [(E)-3-(4-((2-(4-fluoro-2,6-dimethylbenzoyl)-6-hydroxybenzo [b]thiophen-3-yl)oxy)phenyl)acrylic acid, also identified as G1T48], AZD9496 [(E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid], (E)-3-(4-((2-(2-(1,1-difluoroethyl)-4-fluorophenyl)-6-hydroxybenzo[b]thiophen-3-yl)oxy)phenyl)acrylic acid (also identified as LSZ102), D-0502, LY3484356, GDC-0927, SHR9549, and a pharmaceutically acceptable polymorph, enantiomer, stereoisomer, salt, solvate or tautomer of any of the foregoing. In a preferred aspect, the SERD compound is selected from amcenestrant, azenosertib (ZN-c5), borestrant, brilanestrant, camizestrant, elacestrant, fulvestrant, giredestrant, imlunestrant, rintodestrant, AZD9496 (LSZ102), D-0502, LY3484356, GDC-0927 or SHR9549, and a pharmaceutically acceptable polymorph, enantiomer, stereoisomer, salt, solvate or tautomer of any of the foregoing.

The term “aromatase Inhibitor (“AI”)” designates a compound that reduces systemic levels of estrogen. The aromatase inhibitors can be a steroidal aromatase inhibitor or a non-steroidal aromatase inhibitor. For example, the one or more aromatase inhibitors can be selected from aminoglutethimide, exemestane (steroidal aromatase inhibitor), testolactone (steroidal aromatase inhibitor), anastazole (non-steroidal aromatase inhibitor), letrozole (non-steroidal aromatase inhibitor), fadrozole (non-steroidal aromatase inhibitor), formestane (steroidal aromatase inhibitor), vorozole (non-steroidal aromatase inhibitor) and AZD9496 (non-steroidal aromatase inhibitor), including any pharmaceutically acceptable polymorph, enantiomer, stereoisomer, salt, solvate or tautomer of any of the foregoing. The AI is preferably selected from aminoglutethimide, anastrozole, exemestane, fadrozole, letrozole, vorozole, AZD9496, and a pharmaceutically acceptable polymorph, enantiomer, stereoisomer, salt, solvate or tautomer of any of the foregoing.

The term “Complete Estrogen Receptor Antagonist (“CERAN”)” designates a compound that potently and completely inactivates the estrogen receptor (ER). The CERAN is preferably OP-1250, ARV-471 ((3S)-3-[6-[4-[[1-[4-[(1R,2S)-6-hydroxy-2-phenyl-1,2,3,4-tetrahydronaphthalen-1-yl]phenyl]piperidin-4-yl]methyl]piperazin-1-yl]-3-oxo-1H-isoindol-2-yl]piperidine-2,6-dione), H3B-6545 ((E)-N,N-dimethyl-4-[2-[5-[(Z)-4,4,4-trifluoro-1-(3-fluoro-2H-indazol-5-yl)-2-phenylbut-1-enyl]pyridin-2-yl]oxyethylamino]but-2-enamide), or N-(2-(4-(Adamantan-2-ylidene(4-hydroxyphenyl)methyl)phenoxy)ethyl)-adamantane-1-carboxamide, or a pharmaceutically acceptable polymorph, enantiomer, stereoisomer, salt, solvate or tautomer of any of the foregoing. Additional examples of similar CERAN compounds are described in WO19/241231.

The term inhibitor of the cell cycle designates a compound that is designed to slow down or stop cell cycle progression through various mechanisms. Cell cycle arrest can be induced at different stages, decreasing the rate of cell division and the number of actively cycling cells.

The inhibitor of the cell cycle is preferably selected from palbociclib, ribociclib, abemaciclib and a pharmaceutically acceptable polymorph, enantiomer, stereoisomer, salt, solvate or tautomer of any of the foregoing.

The term “growth factor receptor agent” designates a compound that is designed to target the epidermal growth factor receptor (EGFR), fibroblast growth factor receptor (FGFR), insulin growth factor receptor (IGFR) and/or vascular endothelial growth factor receptor (VEGFR).

The term “targeted agent” designates an inhibitor of the cell cycle, of the PI3K/Akt/mTOR pathway, or of a growth factor receptor; and/or a compound targeting alterations in the ubiquitin-proteasome pathway; and/or a compound increasing the activity of the bromodomain and/or extra-terminal domain of a protein.

The targeted agent is preferably selected from palbociclib, ribociclib, abemaciclib, buparlisib, pilaralisib, pictilisib, sonolisib, dactolisib, sapanisertib, voxtalisib, serabelisib, alpelisib, perifosine, MK2206, ipatasertib, GSK690693, temsirolimus, ridaforolimus, sirolimus, everolimus, AZD-8055, OSI-027, lapatinib and a pharmaceutically acceptable polymorph, enantiomer, stereoisomer, salt, solvate or tautomer of any of the foregoing. The targeted agent is even more preferably palbociclib, ribociclib, abemaciclib and/or a pharmaceutically acceptable polymorph, enantiomer, stereoisomer, salt, solvate or tautomer of any of the foregoing.

The term “small molecule” designates in particular a small molecule compound such as for example tipiracil, vistonuridine (uridine triacetate), zoledronic or a pharmaceutically acceptable polymorph, enantiomer, stereoisomer, salt, solvate or tautomer of any of the foregoing.

In the context of the present invention, the term “antibody” (or “immunoglobulin”) designates any kind of antibody such as a monoclonal antibody, a multispecific antibody (i.e. an antibody comprising a first antigen binding site and at least one second different antigen binding site; e.g. a bispecific antibody) or a single chain antibody. This term also covers as any (functional) fragment thereof.

An typical antibody consists of a glycoprotein comprising at least two heavy (H) chains and two light (L) chains inter-connected by disulfide bonds. Each heavy chain comprises a heavy chain variable region (or domain) (abbreviated herein as VH) and a heavy chain constant region (hereafter CH). Heavy chains are classified as gamma, mu, alpha, delta, or epsilon, and define the antibody's isotype as IgG, IgM, IgA, IgD, and IgE, respectively. The heavy chain constant region of the immunoglobulin IgG, IgD, and IgA (γ, δ and α chains respectively) comprises three domains (CH1, CH2, and CH3) and a hinge region for added flexibility, and the heavy chain constant region of the immunoglobulin IgM and IgE contains 4 domains (CH1, CH2, CH3, and CH4). The antibody of the invention can be of the IgG, IgM, IgA, IgD, and IgE isotype, depending on the structure of its heavy chain. However, in a preferred embodiment, the antibody of the invention is of the IgG isotype, i.e., its heavy chain is of the gamma (γ) type.

IgG antibodies are classified in four distinct subtypes, namely IgG1, IgG2, IgG3 and IgG4 in the order of their abundance in serum (IgG1 being the most abundant). The structure of the hinge regions in the γ chain gives each of these subtypes its unique biological profile (even though there is about 95% similarity between their Fc regions, the structure of the hinge regions is relatively different).

The antibody of the invention can be of the IgG1, IgG2, IgG3 or IgG4 subtype. However, in a preferred embodiment, the antibody of the invention is of the IgG1 subtype.

Each light chain comprises a light chain variable region (abbreviated herein as VL) and a light chain constant region comprising only one domain, CL. There are two types of light chain in mammals: the kappa (κ) chain, encoded by the immunoglobulin kappa locus on chromosome 2, and the lambda (λ) chain, encoded by the immunoglobulin lambda locus on chromosome 22. In a preferred embodiment, the antibody of the invention has a Kappa light chain. The VH and VL regions can be further subdivided into regions of hypervariability, termed “Complementarity Determining Regions” (CDR), which are primarily responsible for binding an epitope of an antigen, and which are interspersed with regions that are more conserved, termed “Framework Regions (FR). Each VH and VL is composed of three CDRs and four FRs, arranged from amino-terminus to carboxy-terminus in the following order: FR1, CDR1, FR2, CDR2, FR3, CDR3, FR4. The functional ability of the antibody to bind a particular antigen depends on the variable regions of each light/heavy chain pair, and is largely determined by the CDRs. The variable region of the heavy chain differs in antibodies produced by different B cells, but is the same for all antibodies produced by a single B cell or B cell clone (or hybridome).

By contrast, the constant regions of the antibodies mediate the binding of the immunoglobulin to host tissues or factors, including various cells of the immune system (e.g. effector cells) and the first component (Clq) of the classical complement system.

As used herein, the term “antibody fragments” intends to designate Fab, Fab′, F(ab′)2, scFv, dsFv, ds-scFv, single chain antibody, dimers, minibodies, nanobodies, diabodies, and multimers thereof and bispecific antibody fragments. Antibodies can be fragmented using conventional techniques. Various techniques have been developed for the production of antibody fragments. Traditionally, these fragments were derived via proteolytic digestion of intact antibodies. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, nanobodies, diabodies, bispecific antibody fragments and other fragments can also be synthesized by recombinant techniques.

Typically, the antibody fragment of the invention is a functional fragment, i.e. an antibody fragment capable of binding and preferably inhibiting or neutralizing the activity of a molecule of interest as does the antibody it is deriving from.

In another particular embodiment, the antibody of the invention is a monoclonal antibody.

A “monoclonal antibody”, as used herein, designates an antibody arising from a nearly homogeneous population of antibodies. More particularly, the antibodies of a given subject are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one isotype and subtype, and light chains of only one type. In addition, in contrast with preparations of polyclonal antibodies, each monoclonal antibody is directed to a single epitope of an antigen.

To produce monoclonal antibodies, antibody producing cells (lymphocytes) can be harvested from an immunized animal as described above and fused with myeloma cells by standard somatic cell fusion procedures thereby immortalizing these cells and yielding hybridoma cells. Such techniques are well known in the art (e.g. the hybridoma technique originally developed by Kohler and Milstein (1975)) as well as other techniques such as the human B-cell hybridoma technique, the EBV-hybridoma technique to produce human monoclonal antibodies, and screening of combinatorial antibody libraries. Hybridoma cells can be screened immunochemically for production of antibodies specifically reactive with the target polypeptide(s) so that only monoclonal antibodies binding to said polypeptide(s) are isolated.

The antibody or a fragment thereof of the invention may be a human, chimeric, humanized, murine, CDR-grafted, phage-displayed, bacteria-displayed, yeast-displayed, transgenic-mouse produced, mutagenized, or randomized antibody or fragment.

A chimeric antibody is a molecule in which different portions are derived from different animal species, such as those having a variable region derived from a murine monoclonal antibody (mAb) and a human immunoglobulin constant region.

Humanized forms of antibodies of the invention are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin (recipient antibody) are replaced by corresponding non-human residues of the donor antibody. Furthermore, humanized antibodies may comprise residues that are not found in the recipient antibody or in the donor antibody.

In general, the humanized antibody may comprise substantially all of at least one, typically two, variable domains, in which all or substantially all of the hypervariable loops correspond to those of a non-human immunoglobulin (donor antibody having the desired specificity, affinity, and capacity) and all or substantially all of the FRs are those of a human immunoglobulin sequence. Methods for humanizing non-human antibodies have been described in the art. Preferably, a humanized antibody has one or more amino acid residues introduced into it from a source, which is non-human. These non-human amino acid residues are often referred to as “import” residues, which are typically taken from an “import” variable domain. Humanization may be essentially performed by substituting hypervariable region sequences for the corresponding sequences of a human antibody. Accordingly, such humanized antibodies are chimeric wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. Other methods generally involve conferring donor CDR binding affinity onto an antibody acceptor variable region framework. One method involves simultaneously grafting and optimizing the binding affinity of a variable region binding fragment. Another method relates to optimizing the binding affinity of an antibody variable region.

The antibody or fragment thereof of the invention may be administered in their “naked” or unconjugated form, or may have other agents conjugated to them, such as drug, toxin or radioactive atom.

The “antibody” is for example alemtuzumab, amelimumab, amivantamab, atezolizumab, avelumab, besilesomab, bevacizumab, blinatumomab, brentuximab, catumaxomab, cemiplimab, cetuximab, dalotuzumab, daratumumab, denosumab, dinutuximab, durvalumab, elotuzumab, elranatamab, epratuzumab, farletuzumab, gemtuzumab, inotuzumab ozogamicin, ipilimumab, matuzumab, mogamulizumab, moxetumomab pasudotox, nimotuzumab, PF-07260437, PF-07257876, PF-07062119, necitumumab, nivolumab, obinutuzumab, ocaratuzumab, onartuzumab ofatumumab, olaratumab, oregovomab, panitumumab, pembrolizumab, pertuzumab, racotumomab, ramucirumab, rilotumumab, rituximab, siltuximab, tabalumab, tocilizumab, tomuzotuximab, tositumomab, trastuzumab, zalutumumab, zanolimumab or zenocutuzumab.

The term “antibody-drug conjugate” (“ADC”) designates in particular an ADC compound such as for example trastuzumab emtansine, fam-trastuzumab deruxtecan-nxki (Enhertu®), trastuzumab duocarmazine, disitamab vedotin, ladiratumab vedotin (also known as SGN-LIV1A), depatuxizumab mafodotin, SN-38 or a pharmaceutically acceptable polymorph, enantiomer, stereoisomer, salt, solvate or tautomer of any of the foregoing.

The term “antisense molecule” designates in particular several classes of oligonucleotide molecules that contain sequence complementarity to target RNA molecules, such as mRNA, viral RNA, or other RNA species, and that inhibit the function of their target RNA after sequence-specific binding. The antisense molecule may be selected from an antisense oligodeoxyribonucleotide (ODN), i.e., a single-stranded DNA molecule, a small interfering RNA (siRNA) molecule, a ribozyme, a DNAzyme and a pharmaceutically acceptable polymorph, enantiomer, stereoisomer, salt, solvate or tautomer of any of the foregoing.

Within the context of the present invention, “non-responder” or “resistant” refers to the phenotype of a subject who does not respond to a cancer treatment, in particular to a conventional treatment of cancer as herein identified, i.e., the volume of the tumor does not substantially decrease, or the symptoms of the cancer in the subject are not alleviated, or the cancer progresses, for example the volume of the tumor increases and/or the tumor generates local or distant metastasis. The terms “non-responder” or “resistant” also refer to the phenotype of a subject who will die from the cancer.

Within the context of the present invention, “responder” or “sensitive” refers to the phenotype of a patient who responds to a treatment of cancer, in particular to a conventional treatment of cancer as herein defined, i.e. the volume of the tumor is decreased, at least one of his symptoms is alleviated, or the development of the cancer is stopped, or slowed down.

A subject responding to a cancer treatment is, in the sense of the present invention, a subject who typically has a much longer disease free survival (“DFS”) or metastasis free survival (“MFS”) chance than a patient who has not been identified, with a method as herein described, as sensitive to a treatment of cancer. In a preferred embodiment, a subject who responds to a cancer treatment is a subject who will be completely treated (cured), i.e., a subject who will survive to the cancer [the detected or measured parameter (for example the expression product of gene as herein disclosed) has a beneficial impact on the “overall survival” (OS)].

Within the context of this invention, the term pathological complete response (“pCR”) means that the tumor size dramatically decreases under anti-cancer treatment, typically after a neoadjuvant hormonal therapy, and then becomes operable (possible treatment by surgery) or becomes undetectable.

Within the context of this invention, the term “progressive disease” refers to an at least 20 percent growth in the size of the tumor or spread of the tumor since the beginning of treatment. In other words, if the size of a tumor is 20 percent larger on a scan than that originally measured, it would be called progressive disease.

The terms “primary endocrine resistance” or “primary hormonal resistance” used to characterize a patient refer to the patient's relapse on adjuvant hormone therapy within the first two years, or to progressive disease within the first 6 months on first-line hormonal therapy in the metastatic setting.

The terms “secondary endocrine resistance” or “secondary hormonal resistance” used to characterize a patient refer to the patient's relapse on adjuvant hormonal therapy after 2 years, to the patient's relapse within the first 12 months of completing adjuvant therapy, or to progressive disease 6 months after initiating endocrine therapy in the metastatic setting.

The term “early relapse” refers to a relapse occurring less than 12 months on adjuvant endocrine therapy.

The term “late relapse” refers to a relapse occurring more than 12 months on adjuvant therapy.

The term “sample” refers to a material or mixture of materials containing one or more components of interest. A sample from a subject refers to a sample obtained from the subject, including samples of biological tissue or fluid origin, obtained, reached, or collected in vivo or in situ. A sample can be obtained from a region of a subject containing precancerous or cancer cells or tissues or from another tissue or fluid in the subject. Such samples can be, but are not limited to, organs, tissues, fractions and cells isolated from a mammal. Exemplary samples include lymph node, whole blood, partially purified blood, serum, plasma, bone marrow, and peripheral blood mononuclear cells (“PBMC”). A sample can be also a tissue biopsy. Exemplary samples also include cell lysate, a cell culture, a cell line, a tissue, an organ, a biological fluid, a blood sample, a urine sample, and the like.

The sample preferably includes tumor cells or tumoral nucleic acid, for example DNA or RNA. The tumoral DNA may be for example cell-free DNA (“cfDNA”) or circulating tumor DNA (“ctDNA”). cfDNA may be found, for example, in a blood sample of the subject. ctDNA may be found, for example, in a plasma sample of the subject.

A sample from a subject can have multiple copies of the ESR1 gene. These copies can encode wild type and/or mutant ERa proteins. As used herein, a sample from a subject having an ESR1 mutation can also have one or more copies of the wild type ESR1 gene and/or the wild type ERa protein.

The term “about” which is used to modify a numerically defined parameter means that the parameter may vary by as much as 10% above or below the stated numerical value for that parameter. For example, a dose of “about 5 mg” means 5 mg±10%, i.e., the dose may vary between 4.5 mg and 5.5 mg.

As used herein, an “effective dosage” or “effective amount” of a compound, combination or composition is an amount sufficient to affect any one or more beneficial or desired outcomes, including biochemical, histological and/or behavioral symptoms, of the disease, its complications and intermediate pathological phenotypes presenting during development of the disease.

As used herein, an “effective dosage” or “effective amount” of a compound or pharmaceutical composition is an amount sufficient to affect any one or more beneficial or desired outcomes, including biochemical, histological and/or behavioral symptoms, of the disease, its complications and intermediate pathological phenotypes presenting during development of the disease.

For therapeutic use, a “therapeutically effective amount” refers to that amount of a compound or combination being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or (7) delaying the progression of the disease in a patient.

An effective dosage can be administered in one or more administrations. For the purposes of this invention, an effective dosage of drug, compound, combination or pharmaceutical composition is an amount sufficient to accomplish prophylactic or therapeutic treatment either directly or indirectly. As is understood in the clinical context, an effective dosage of a drug, compound or pharmaceutical composition may or may not be achieved in conjunction with another drug, compound, combination or pharmaceutical composition.

As used herein, a “pharmaceutically acceptable carrier” refers to a carrier, excipient or diluent that does not cause significant irritation to an organism and does not abrogate the biological activity and properties of the active compound or therapeutic agent. The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form. Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes.

Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

As used herein, the terms “combination” or “combination therapy” refer to the administration of each therapeutic agent of the combination therapy of the invention, either alone or in the form of a pharmaceutical composition or medicament, either sequentially, concurrently, or simultaneously.

As used herein, the term “sequential” or “sequentially” refers to the administration of each therapeutic agent of the combination therapy of the invention, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

As used herein, the term “concurrently” refers to the administration of each therapeutic agent in the combination therapy of the invention, either alone or in separate medicaments, the second therapeutic agent being administered immediately after the first therapeutic agent, and the therapeutic agents being administered in any order. In a preferred embodiment the therapeutic agents are administered concurrently.

As used herein, the term “simultaneous” refers to the administration of each therapeutic agent of the combination therapy of the invention in the same medicament. As will be understood by those skilled in the art, the combination therapy may be usefully administered to a subject during different stages of their treatment.

In some embodiments of each of the methods, combinations and uses herein, the combination therapy is administered to a subject who is previously untreated, i.e. is treatment naive.

In some preferred embodiments of each of the methods, combinations and uses herein, the combination therapy is administered to a subject who has failed to achieve a sustained response after at least one prior therapy with an anti-cancer agent as herein identified, i.e. is a treatment experienced-subject.

### Quinazoline Carboxamide Azetidine Compounds

The quinazoline carboxamide azetidine compounds useful in the context of the present invention, in particular in the treatment of a hormone dependent disease, in particular of a cancer as herein described, preferably of breast cancer, are compounds defined by Formula (I):

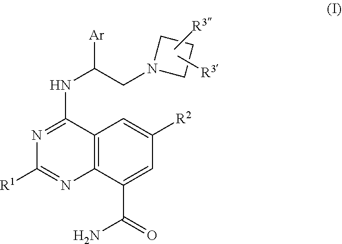

and/or any pharmaceutically acceptable polymorph, enantiomer, stereoisomer, salt, solvate or tautomer thereof, including any mixture thereof in all ratios, wherein:
R¹ is H or LA;
R² is Hal, O(LA), N(LA)(LA)′, CONH(LA), Ar, CONH₂ or A;
R^{3′}, R^{3″} independently are H, LA or Hal;
Ar is a mono- or bicyclic aromatic homo- or heterocycle having 0, 1, 2, 3 or 4 N, O and/or S atoms and 5, 6, 7, 8, 9, or 10 skeleton atoms, which may be unsubstituted or, independently of one another, mono-, di- or trisubstituted by Hal, A, Art, OH, SH, OA, O(Ar1), NH₂, NHA, NH(Ar1), NA₂>NO₂, CN, OCN, SCN, COOH, COOA, CONH₂, CONHA, CONH(Art), CONA₂, NHCOA, NHCO(Art), NHCONHA, NHCONH(Art), NHCONH₂, NHSO₂A, NHSO₂(Ar1), COA, CO(Ar1), SO₂NH₂, SO₂A, SO₂(Ar1) and/or SO₂Hal, and in which a ring N-atom may be substituted by an O-atom to form an N-oxide group, and in which in the case of a bicyclic aromatic cycle on one of the two rings may be partly saturated; Ar1 is a monocyclic aromatic homo- or heterocycle having 0, 1, 2 or 3 N, O and/or S atoms and 5 or 6 skeleton atoms, which may be unsubstituted or, independently of one another, mono-, di- or trisubstituted by Hal, LA, OH, SH, O(LA), NH₂, NH(LA), N(LA)₂, NO₂, CN, OCN, SCN, COOH, COO(LA), CONH₂, CONH(LA), CON(LA)₂, NHCO(LA), CHO, CO(LA), SO₂NH₂, SO₂(LA) and/or SO₂Hal;
A is unbranched or branched linear or cyclic alkyl having 1, 2, 3, 4, 5, 6, 7 or 8 C atoms, in which one or two CH₂ groups may be replaced by an O or S atom and/or by an —NH—, —CO—, —NHCOO—, —NHCONH—, —N(LA)-, —CONH—, —NHCO— or —CH═CH— group, and in which 1-3H atoms may be replaced by Hal, and in which one or two CH₃ groups may be replaced by OH, SH, NH₂, NH(LA), N(LA)₂, NHCOOH, NHCONH₂ or CN;
LA is unbranched or branched, linear alkyl having 1, 2, 3 or 4 C atoms, wherein 1, 2 or 3H atoms may be replaced by Hal, e.g., methyl, ethyl, trifluoromethyl, difluoromethyl, 1,1,1-trifluoroethyl, propyl, isopropyl, butyl, isobutyl, sec-butyl or tert-butyl; and
Hal is F, Cl or Br, preferably F or Cl, most preferably F.

A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2.2-dimethylpropyl, 1-ethylpropyl, hexyl, 1-, 2-, 3- or 4-methylpentyl, 1,1-, 1,2-, 1.3-, 2, 2—, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl.

A further preferably denotes alkyl as defined above, in which one or two CH₂ groups may be replaced by O or S atoms and/or by NH, N(LA), CONH, NHCO or —CH═CH— groups and/or in addition 1-3H atoms may be replaced by F and/or CI, such as, for example, trifluoromethyl, pentafluoroethyl, 1,1-difluoromethyl, 1,1,1-trifluoroethyl, methoxy, ethoxy, n-propoxy, isopropoxy, n-butoxy, isobutoxy, sec-butoxy or tert-butoxy.

In a preferred embodiment, the novel quinazoline carboxamide azetidine compounds are further defined by Formula (II):

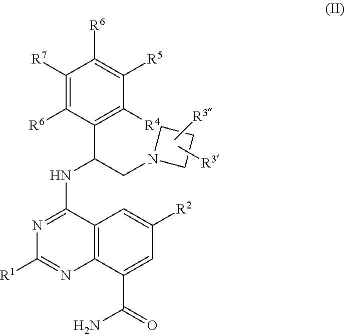

and/or any pharmaceutically acceptable polymorph, enantiomer, stereoisomer, salt, solvate or tautomer thereof, including any mixtures thereof in all ratios, wherein:
R⁴, R⁵, R⁶, R⁷, R⁸, independently are H, Hal, LA, OH, SH, O(LA), NH₂, NH(LA), N(LA)₂, NO₂, CN, OCN, SCN, COOH, COO(LA), CONH₂, CONH(LA), CON(LA)₂, NHCO(LA), NHCONH(LA), NHCONH₂>NHSO₂(LA)(CO(LA), SO₂NH₂, SO₂(LA) or SO₂Hal,
R⁵, R⁶ together with the phenyl group they are attached to, may form a 9 or 10 membered bicyclic ring system, in which 1 or 2 of the non-phenyl carbon atoms may be independently replaced by NH, O or S, in which the cycle formed by R⁵ and R⁶ may be unsubstituted or mono- or disubstituted by Hal or LA, one of R⁵, R⁶, R⁷ may be AM, O(Ar1), NH(A), CONH(Ar1), NHCO(Ar1), NHCONH(Ar1), NHSO₂(Ar1), CO(Ar1) or SO₂(Ar1), while the other two of R⁵, R⁶, R⁷ are not Ar1, O(Ar1), NH(Ar1), CONH(A), NHCO(Ar1), NHCONH(Ar1), NHSO₂(Ar1), CO(Ar1) or SO₂(Ar1), and the remaining substituents have the meanings indicated for Formula (I).

In a more preferred embodiment of Formulae (I) and (II), the stereochemistry at the central chiral carbon atom is as shown in Formulae (I′) and (II′):

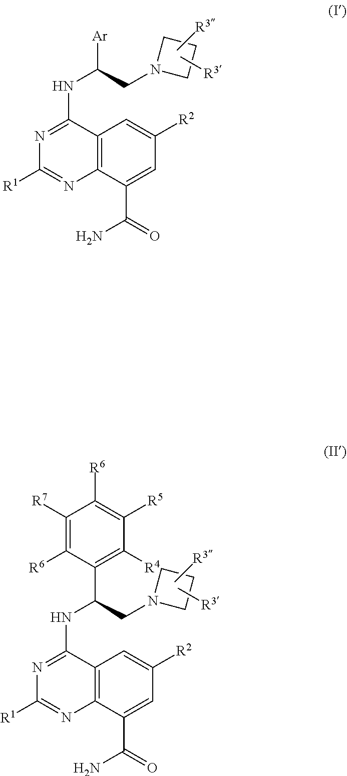

In general, all residues which occur more than once may be identical or different, i.e., are independent of one another. Above and below, the residues and parameters have the meanings indicated for Formula (I), Formula (II), Formula (I′) and Formula (II″) unless expressly indicated otherwise.

Further preferred are compounds of subformulae 1 to 12 of Formulae (II) and (II′), wherein in Subformula 1:
R⁴, R⁵, R⁶, R⁷, R⁸ independently are H, F, Cl, Br, OH, LA, O(LA), CN, C(Hal)₃, OC(Hal)₃
in Subformula 2:
R¹, R² are H
in Subformula 3:
R^{3′}, R^{3″} independently are H, OH or F in Subformula 4:
R⁴, R independently are H, F or CI
in Subformula 5:
R⁵, R⁷ independently are H, F, Cl, Br, CN, methoxy or CF₃
in Subformula 6:
R⁵, R⁶ together with the phenyl group they are attached to form benzo-1,2-dioxolyl, of which the carbon atom bridging the two oxygen atoms may be unsubstituted, or mono- or disubstituted by F or methyl in Subformula 7:
R⁶ is H, F, CI or CF₃
in Subformula 8:
R⁵, R⁶ independently are H, F, Cl, Br, methyl, CHF₂ or CF₃
in Subformula 9:
R¹, R², R^{3′}, R^{3″}, R⁴, R⁷, R⁸ are H
in Subformula 10:
R¹, R², R^{3′}, R^{3″}, R⁴, R⁷, R⁸ are H,
R⁵, R⁶ are independently H, F, Cl, Br, methyl, CHF₂ or CF₃
in Subformula 11:
R¹, R², R^{3′}, R^{3″}, R⁴, R⁸ are H,
R⁵ is Br, methyl, CHF₂ or CF₃,
R⁶ is F, Cl or CF₃,
R⁷ is H or F
in Subformula 12:
R¹, R², R⁴, R⁸ are H,
R^{3′} is F, or methyl,
R^{3″} is H,
R⁵ is Br, methyl, CHF₂ or CF₃,
R⁶ is F, Cl or CF3,
R⁷ is H or F,
and the remaining residues have the meaning as indicated for Formula (I).

The compounds of the Formula (I), Formula (II), Formula (I′) and Formula (II′) may have one or more centers of chirality. They may accordingly occur in various enantiomeric forms and be in racemic or optically active form. The invention therefore also relates to the optically active forms (stereoisomers), the enantiomers, the racemates, and the diastereomers of these compounds. Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic enantio-mer resolution with the aid of an optically active resolving agent (for example dinitrobenzoylphenylglycine, cellulose triacetate or other derivatives of carbohydrates or chirally derivatised methacrylate polymers immobilized on silica gel). Suitable eluents for this purpose are aqueous or alcoholic solvent mixtures, such as, for example, hexane/isopropanol/acetonitrile, for example in the ratio 82:15:3. An elegant method for the resolution of racemates containing ester groups (for example acetyl esters) is the use of enzymes, in particular esterases.

The compounds of the present invention can be in the form of a prodrug compound. “Prodrug compound” means a derivative that is converted into a biologically active compound according to the present invention under physiological conditions in the living body, e.g., by oxidation, reduction, hydrolysis or the like, each of which is carried out enzymatically, or without enzyme involvement.

Examples of prodrugs are compounds, wherein the amino group in a compound of the present invention is acylated, alkylated or phosphorylated, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated, or wherein a sulfhydryl group forms a disulfide bridge with a carrier molecule, e.g. a peptide, that delivers the drug selectively to a target and/or to the cytosol of a cell. These compounds can be produced from compounds of the present invention according to well-known methods. Other examples of prodrugs are compounds, wherein the carboxylate in a compound of the present invention is for example converted into an alkyl-, aryl-, choline-, amino-, acyloxymethylester, or linolenoyl-ester.

Metabolites of compounds of the present invention are also within the scope of the present invention.

Where tautomerism, e.g., keto-enol tautomerism, of compounds of the present invention or their prodrugs may occur, the individual forms, e.g., the keto or the enol form, are claimed separately and together as mixtures in any ratio. The same applies for stereoisomers, e.g., enantiomers, cis/trans isomers, conformers and the like.

If desired, isomers can be separated by methods well known in the art, e.g. by liquid chromatography. The same applies for enantiomers, e.g., by using chiral stationary phases. Additionally, enantiomers may be isolated by converting them into diastereomers, i.e., coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials.

The compounds of the present invention can be in the form of a pharmaceutically acceptable salt or a solvate.

The term “pharmaceutically acceptable salts” refers to salts prepared from pharmaceutically acceptable non-toxic bases or acids, including inorganic bases or acids and organic bases or acids. In cases where the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present in salt form, and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the present invention which contain one or more basic groups, i.e. groups which can be protonated, can be present in salt form, and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to a person skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable solvates” means addition forms with pharmaceutically acceptable solvents that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, e.g. a mono- or dihydrate.

If the solvent is alcohol, the solvate formed is an alcoholate, e.g., a methanolate or ethanolate. If the solvent is an ether, the solvate formed is an etherate, e.g., diethyl etherate.

Therefore, the following items are also in accordance with the invention:
a) all stereoisomers or tautomers of the compounds, including mixtures thereof in all ratios,
b) prodrugs of the compounds, or stereoisomers or tautomers of these prodrugs,
c) pharmaceutically acceptable salts of the compounds and of the items mentioned under (a) and (b),
d) pharmaceutically acceptable solvates of the compounds and of the items mentioned under (a), (b) and (c).

It should be understood that all references to compounds above and below are meant to include these items, in particular pharmaceutically acceptable solvates of the compounds, or pharmaceutically acceptable solvates of their pharmaceutically acceptable salts.

In the context of the present invention, a preferred quinazoline carboxamide azetidine compound is 4-[(S)-2-Azetidin-1-yl-1-(4-chloro-3-trifluoromethyl-phenyl)-ethylamino]-quinazoline-8-carboxylic acid amide (also herein identified as “M2698”).

Quinazoline carboxamide azetidine compound used in the present invention, such as compounds of formulae (I), (II), (I′) and (II′) as above defined, and preferably M2698, are described further in the international patent application WO2012/69146, “Quinazoline carboxamide azetidines”. A skilled person can refer to this patent application for the synthesis of these quinazoline carboxamide azetidine compounds.

The present invention also provides a composition comprising a quinazoline carboxamide azetidine compound, preferably M2698. In a particular embodiment, the composition comprises a quinazoline carboxamide azetidine compound, preferably M2698, and a pharmaceutically acceptable excipient, carrier, or diluent.

### Additional Therapeutic Agents, Preferably Anti-Cancer Agents

As taught herein above, in a preferred aspect, the combination or composition comprises, in addition to the quinazoline carboxamide azetidine compound, in particular in addition to 4-(S)-2-Azetidin-1-yl-1-(4-chloro-3-trifluoromethyl-phenyl)-ethylamino]-quinazoline-8-carboxylic acid amide, or to a pharmaceutically acceptable polymorph, enantiomer, stereoisomer, salt, solvate or tautomer thereof, at least one distinct therapeutic agent, preferably anti-cancer agent.

In a preferred embodiment, the at least one distinct therapeutic agent, preferably anti-cancer agent, is an antineoplastic agent, a signal transduction inhibitor or a mixture thereof, as herein described in the “definition” section.

The combination or composition of the invention may comprise one or more additional distinct therapeutic agents, preferably anti-cancer agents (also herein identified as agent (c)), such as in particular an endocrine/hormonal therapeutic agent, an anti-angiogenic agent, a signal transduction inhibitor, an antineoplastic agent, a small molecule, an antibody or a fragment thereof, an antibody-drug conjugate (ADC) and an antisense molecule as herein described in the “definition section”, preferably an anti-angiogenic agent, a signal transduction inhibitor, an antineoplastic agent, a small molecule, a growth factor receptor agent, an antibody or a fragment thereof, an antibody-drug conjugate (ADC) or an antisense molecule as herein described, wherein the amounts are together effective in treating the cancer.

In some embodiments, the additional anti-cancer agent is selected from the group consisting of abemaciclib (LY2835219), abiraterone, abiraterone acetate, abivertinib, acalabrutinib, aclarubicin, aldesleukin (IL-2), ado-trastuzumab emtansine, afatinib dimaleate, afimoxifene, afuresertib, alectinib, alemtuzumab, altretamine, almonertinib, aloisine A, aloisine B, alpelisib, alsterpaullone, amifostine, amcenestrant (SAR439859), amelimumab, aminoglutethimide, aminopurvalanol, anastrozole, amivantamab, apalutamide, apaziquone, aprepitant, arsenic trioxide, arzoxifene, asparaginase (pegaspargase), atezolizumab, atorvastatin (Caduet, Lipitor, Lypqozet), avapritinib, avelumab, axicabtagene ciloleucel, axitinib, azacytidine, azathioprine, azenosertib (ZN-c5), bazedoxifene, belinostat, bendamustine hydrochloride, bevacizumab, bexarotene, bicalutamide, bimiralisib, binimetinib (Mektovi or ARRY-162), birociclib (XZP 3287), bleomycin sulfate, blinatumomab, bohemine, borestrant, bortezomib, borussertib, bosutinib, brentuximab vedotin, brigatinib, brilanestrant (GDC-0810), buparlisib (BKM120), busulfan, butyrolactone, cabazitaxel, cabozantinib-s-malate, calaspargase pegol-mknl, camizestrant (AZD9833), capecitabine, capivasertib (AZD5363), caplacizumab-yhdp, capmatinib hydrochloride, carboquone, carboplatin, carfilzomib, carmustine, cemiplimab-rwlc, celecoxib, celecoxib derivative, cemiplimab, ceritinib, cetuximab, chlorambucil, chlormethine, cilengitide cisplatin, cladribine, clofarabine, cobimetinib, copanlisib hydrochloride, crizotinib, cyclophosphamide, cyproterone, cytarabine, dabrafenib mesylate, dacarbazine, dacomitinib, dactinomycin, dactolisib tosylate, dalpiciclib (SHR-6390), daratumumab, daratumumab and hyaluronidase-fihj, darbepoetin alfa, darolutamide, dasatinib, daunorubicin hydrochloride, decitabine, defibrotide sodium, degarelix, denileukin, depatuxizumab mafodotin, detorsertib, diftitox, denosumab, dexamethasone, dexrazoxane hydrochloride, dichloroacetate, dinaciclib, dinutuximab, disitamab vedotin (RC48-ADC), docetaxel, dordaviprone (ONC-201), dovitinib, doxorubicin hydrochloride, droloxifene, durvalumab, duvelisib, elacestrant, elotuzumab, eltanexor, eltrombopag olamine, elranatamab, emapalumab-Izsg, eltanexor (KPT-8602), enasidenib mesylate, encorafenib, enfortumab vedotin-ejfv, endoxifen, entrectinib, enzalutamide, enzastaurin, epertinib, epirubicin hydrochloride, epoetin alfa, eptaplatin, erdafitinib, eribulin mesylate, erlotinib hydrochloride, etoposide, etoposide phosphate, everolimus, exemestane, fadrozole, fedratinib hydrochloride (Filgrastim®), flavopiridol, floxuridine, fludarabine phosphate, fluorouracil, 5-fluorouracil, flutamide, formestane, fosbretabulin, fostamatinib disodium, fotemustine, fulvestrant, gefitinib, gemcitabine hydrochloride, gemtuzumab ozogamicin, gingerenone, gilteritinib fumarate, giredestrant (GDC9545), glasdegib maleate, glucarpidase, glufosfamide, goserelin acetate, granisetron, granisetron hydrochloride, histrelin, hydroxyurea, ibntumomab tiuxetan, ibrutinib, icotinib, idarubicin hydrochloride, idelalisib, idoxifene, ifosfamide, imatinib mesylate, imiquimod, improsulfan tosilate, indirubin, indirubin-3′-monoxime, infigratinib (BGJ398), inotuzumab ozogamicin, interferon alfa-2b recombinant, interferon gamma, iobenguane I-131, ipatasertib, ipilimumab, irinotecan hydrochloride, isatuximab-irfc, ivosidenib, ixabepilone, ixazomib citrate, kenpaullone, ketoconazole, ladiratumab vedotin (also known as SGN-LIV1A), lanreotide acetate, lapatinib ditosylate, ladiratumab vedotin (SGN-LIV1A), larotrectinib sulfate, larotinib, larotrectinib, lasofoxifene, lazertinib, lenalidomide, lenvatinib mesylate, letrozole, lerociclib (G1T38), leucovorin calcium, leuprolide acetate, levamisole, lifirafenib, lobaplatin, lomustine, lonafarnib, lorlatinib, lucitanib, lurbinectedin, luteinizing Hormone-Releasing Hormone (LHRH) agonist, marizomib, mechlorethamine, medroxyprogesterone acetate, megestrol acetate, melphalan, melphalan hydrochloride, mercaptopurine, meriolin 3, methotrexate, midostaurin, milciclib, miltefosine, mitobronitol, mitolactol, miriplatine hydrate, miransertib, mitomycin, mitotane, mitoxantrone hydrochloride, mobocertinib, mogamulizumab, mogamulizumab-kpkc, monepantel, moxetumomab pasudotox-tdfk, naquotinib, narazaciclib (ON123300), nazartinib, necitumumab, nedaplatin, nelarabine, neratinib maleate, nilotinib, nilutamide, nimotuzumab, nimustine, niraparib tosylate monohydrate, nivolumab, obinutuzumab, octreotide, ofatumumab, olaparib, olaratumab, olmutinib, olomoucine, olomoucine II, omacetaxine omepesuccinate, omipalisib, omomyc, ondansetron hydrochloride, onatasertib, osimertinib mesylate, oxaliplatin, paclitaxel, paclitaxel albumin-stabilized nanoparticle formulation, palbociclib (Ibrance, PD-0332991 or PF-00080665), palifermin, palifosfamide, palonosetron hydrochloride, pamidronate disodium, panitumumab, panobinostat, paxalisib, pazopanib hydrochloride, pegaspargase, pegfilgrastim, peginterferon alfa-2b, pembrolizumab, pemetrexed disodium, pemigatinib, pertuzumab, pexidartinib hydrochloride, pictilisib (GDC0941), pimasertib, piperazinyl-pyrimidine derivatives (PF-4708671, PF-4708671), plerixafor, picoplatin, pipindoxifene, pipobroman, plicamycin, polatuzumab vedotin-piiq, pomalidomide, ponatinib hydrochloride, pralsetinib, poziotinib, pralatrexate, prednisone, pralsetinib, procarbazine hydrochloride, propranolol hydrochloride, purvalanol A, purvalanol B, radium 223 dichloride, pyrotinib, raloxifene hydrochloride, ramucirumab, ranimustine, rapamycin analogue, rasburicase, ravulizumab-cwvz, recombinant interferon alfa-2b, refametinib, regorafenib, ribavirin, ribociclib (LEE-011), ridaforolimus, rintodestrant, ripretinib, rituximab, rolapitant, riviciclib hydrochloride (P276-00), roniciclib, romidepsin, romiplostim, R-roscovitine, rucaparib camsylate, ruxolitinib phosphate, sacituzumab govitecan-hziy, sapanisertib, sapitinib, satraplatin, selatinib, selinexor, selpercatinib, selumetinib sulfate, Streptomyces sp OA293, streptozocin, siltuximab, sipuleucel-t, sonalisib, sonidegib, sirolimus, sirotinib, somatostatine (Pasireotide), sorafenib tosylate, streptomyces sp OA293, streptozocin, sunitinib, tagraxofusp-erzs, talazoparib tosylate, talimogene laherparepvec, tamoxifen, tazemetostat hydrobromide, telotristat, temozolomide, temsirolimus, teniposide, tepotinib, tesevatinib, tesetaxel, testolactone, thalidomide, thioguanine, ThioTEPA (N,N′N′-triethylenethiophosphoramide), tipifarnib, tipracil, tisagenlecleucel, tocilizumab, tomuzotuximab, topotecan hydrochloride, toremifene, tositumomab, trabectedin, trametinib, trastuzumab, trastuzumab deruxtecan (Enhertu®), trastuzumab duocarmazine (SYD985), trastuzumab and hyaluronidase-oysk (Herceptin HylectaT^{M}), trastuzumab emtansine, treosulfan, trilaciclib (GTI128), trifluridine and tipiracil hydrochloride, trimetrexate, triptorelin, trofosfamide, tucatinib, ulixertinib, uprosertib (GSK2141795), uramustine valrubicin, vandetanib, varlitinib, vemurafenib, venetoclax, vinblastine, vinblastine sulfate, vindesine, vincristine sulfate, vinflunine, vinorelbine tartrate, vismodegib, vistonuridine (uridine triacetate), vitusertib, vorinostat, vorozole, voruciclib, xylocydin, yinlitinib, zanubrutinib, ziv-aflibercept, zenocutuzumab, Ziv-aflibercept (Zaltrap®), zotatifin, zoledronic acid, zorifertinib, zotatifin, A77 1726 (active metabolite of leflunomide), ABBV-075, ABBV-744, ABP-1119, ABP-1130, AD80/AD81, AG024322, AG-101, AI-6802, AL-5880S, AL-58922, ALM-301, AM-105, AMX-3009, APL-1898, ARQ-751, ARV-471, ASK-120067, ASN-007, AST-2818, ASTX-029, AT-13148, AT7519, ATG-017, AUM-302, AZ6197, AZD4547, AZD5153, AZD5438, AZD5597, AZD8055, AZD9496, BAY-1125976, BAY-1163877, BAY-2476568, BAY-293, BBP-398, BBT-176, BDTX-189, BEBT-108, BEBT-109, BH-2922, BI-1701963, BI-3406, BI-4020, BIERKi, BLU-222, BLU-4810, BMS-387032, BMS-SCH, BMX-002, BO-1978, BPI-15086, BPI-27336, BPI-7711, BR790, BX795, BX912, C-005, CA-102, CA-103, CC-115, CC-223, CC-90003, CK-101, CLM-29, CLM-3, CMAB-017, COTI-2, CR-13626, CSHEGF-29, CT-365, CYC065, D-0316, D-0502, DBPR-112, DC-120, DFN-S29, DGD-1202, DHM-ZS, DS-2087b, DTRMWXHS-12, DZD-9008, EO-1001, ERAS-007, ERAS-601, ES-072, ETH-155008, ETS-001, FCN-411, FCN-437c, FHND-9041, FL772, FLAG-001, FLAG-003, FmAb-2, FP-208, FS-115, FT-I S18, FXY-1, GB-263, GC-1118A, GDC-0927, GLR2007, GSK470, HA-12128, HBI-2376, HEC-68498, HH-2710, HMPL-295, HMPL-309, HMPL-813, HS-627, H3B-6545, IACS-13909, I-BET151, I-BET762, ICP-189, INCB054329, IPA3, IPN-ERK, JAB-3068, JAB-3312, JMT-101, JNJ-7706621, JQ1, JRF-103, JRF-108, JRP-890, JRP-890, JS-111, JSI-1187, JX06, JZB-29, KBP-5209, KNP-501, KO-947, KS-99, KU-004, LL-191, LSZ102, LTT462, LXI-15029, LY2503029, LY2584702, LY2780301, LY3214996, LY3484356, LYS6K2, M074-2865, MCLA-129, MCLA-158, MDC-22, ME-344, MK-0752, MK-2206, MK-8353, MP 0274, mRX-7, MS417, MTX-211, MVC-101, NISC-6, NRC-2694, NSC-765844, NT-004, NT-113, NVP-LCQ19, OBX-1012, OP-1250, ORIC-114, OSI-027, OSU-03012, OSU-53, OT-043, OTX015, PB-357, PF-06821497, PF-06873600, PF-07062119, PF-07104091, PF-07220060, PF-07257876, PF-07260437, PF-07265028, PF-07284 892, PF-0779954, PF-4708671, PHA-793887, PHT-427, PQR-514, PTX-200, PTX-367, QL-1 105, QL-1203, QR-213, R-CR8, RG6171, RGB286147, RGB-286638, RLY-1971, RMC-4550, RMC-4630, RMC-5552, RMC-5845, Ro4584820, RVX-208, RX-0201, RX-0301, RXDX-105, RX-SHP2i, SAH-EJ1, SBF1, SCH772984, SCT-200, SDGR-5, SH3809, SHPO99, SHR9549, SI-2, SJ432, SKLB-1028, SKLB-1206, SN-202, SN-38, SPH-118811, SPR-965, SRX-3177, SYN-004, TAM-03, TAS-117, TAS-6417, TAS-ASTX, TGO2, TGRX-360, TK216, TNO-155, TQB3303, TQB-3804, UBP-1215, VRN-071918, VRN-6, VS-6766, WBP-297, WJ-13404, WSD-0922, WXFL-10030390, X-37-SHP2, XP-105, XZP-5809, YZJ-0318, ZK304709, ZNE-4, ZR-2002, ZSP-0391, ZW49, 10Z-Hymenialdisine, 5-Iodo-indirubin-3′-monoxime or free base, and a pharmaceutically acceptable polymorph, enantiomer, stereoisomer, salt, solvate or tautomer form of any of the foregoing or any combination thereof. The additional anti-cancer agent may also be samuraciclib (also known as CT7001 or ICE0942).

In a preferred aspect, the additional anti-cancer agent is selected from the group of compounds listed herein above, wherein said list does not include afatinib dimaleate, brigatinib, cetuximab, erlotinib hydrochloride, gefitinib, icotinib, simertinib, pimasertib and/or tepotinib.

### Pharmaceutical Compositions and Combinations

A “pharmaceutical composition” refers to a mixture of one or more of the therapeutic agents described herein, or a pharmaceutically acceptable polymorph, enantiomer, stereoisomer, salt, solvate, tautomer, hydrate or prodrug thereof as an active ingredient, and at least one pharmaceutically acceptable carrier or excipient. In some embodiments, the pharmaceutical composition comprises two or more pharmaceutically acceptable carriers and/or excipients.

Thus, “pharmaceutical composition” typically means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention, typically in a therapeutically effective amount, preferably at least (a) a quinazoline carboxamide azetidine compound of formula (I) such as 4-[(S)-2-Azetidin-1-yl-1-(4-chloro-3-trifluoromethyl-phenyl)-ethylamino]-quinazoline-8-carboxylic acid amide (or a pharmaceutically acceptable polymorph, enantiomer, stereoisomer, salt, solvate or tautomer thereof, including any mixture thereof in all ratios) and (b) a distinct therapeutic agent, preferably an antineoplastic agent and/or a signal transduction inhibitor, and pharmaceutically acceptable carrier(s). A pharmaceutical composition of the present invention may additionally comprise one or more other compounds as active ingredients, such as one or more additional therapeutic, preferably anti-cancer, agent (“(c)”) of the present invention, or a prodrug compound or other known substance active against the hormone dependent disease, preferably against cancer.

The (pharmaceutical) combinations or pharmaceutical compositions of the invention include combinations and compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the condition(s) being treated and on the nature of the active ingredient. They may be conveniently presented in unit dosage form and prepared by any of the methods well-known in the art of pharmacy.

The (pharmaceutical) combination or pharmaceutical composition may be, for example, in a form suitable for oral administration as a tablet, capsule, pill, powder, sustained release formulation, solution or suspension, for parenteral injection as a sterile solution, suspension or emulsion, for topical administration as an ointment or cream, or for rectal administration as a suppository.

Exemplary parenteral administration forms include solutions or suspensions of an active compound in a sterile aqueous solution, for example, aqueous propylene glycol or dextrose solutions. Such dosage forms may be suitably buffered, if desired.

The combination or pharmaceutical composition may be in unit dosage forms suitable for single administration of precise amounts.

Combination or pharmaceutical compositions suitable for the delivery of the therapeutic agents of the combination therapies of the present invention, and methods for their preparation will be readily apparent to those skilled in the art. Such compositions and methods for their preparation may be found, for example, in ‘Remington's Pharmaceutical Sciences’, 19th Edition (Mack Publishing Company, 1995), the disclosure of which is incorporated herein by reference in its entirety.

Therapeutic agents of the combination therapies of the invention may be administered orally. Oral administration may involve swallowing, so that the therapeutic agent(s) enters the gastrointestinal tract, or buccal or sublingual administration may be employed by which the therapeutic agent(s) enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups, and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Therapeutic agents of the combination therapies of the present invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the therapeutic agent(s) may make up from 1 wt % to 80 wt % of the dosage form, more typically from 5 wt % to 60 wt % of the dosage form. In addition to the active agent(s), tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch, and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives, and taste-masking agents. Exemplary tablets may contain up to about 80 wt % active agent, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release.

Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al., Pharmaceutical Technology On-line, 25(2), 1-14 (2001). The use of chewing gum to achieve controlled release is described in WO 2000/035298. The disclosures of these references are incorporated herein by reference in their entireties.

The kits described herein may be particularly suitable for administering different dosage forms, for example, oral and parenteral, for administering the separate active (in particular therapeutic) agents of the combination or compositions at different dosage intervals, or for titrating the active (in particular therapeutic) agents of the combination or compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like. The kit may further comprise any other material that may be useful in stratifying prognosis of metastatic breast cancer by identifying tumors with evolving resistance to endocrine therapy, such as a PCR test.

A particular kit comprises i) (a) a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a distinct therapeutic agent as herein described such as an antineoplastic agent and/or a signal transduction inhibitor in different containers, or a composition comprising the combination of (a) and (b) and a pharmaceutically acceptable carrier, and ii) material(s) for administering compounds (a) and/or (b).

A more particular kit comprises i) (a) a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a distinct therapeutic agent as herein described such as an antineoplastic agent as for example a Selective ER modulator (“SERM”), a Selective ER down-regulator/degrader (“SERD”), an aromatase Inhibitor (“AI”), a Complete Estrogen Receptor Antagonist (CERAN), in different containers, or a composition comprising the combination of (a) and (b) and a pharmaceutically acceptable carrier, and ii) material(s) for administering compounds (a) and/or (b).

An even more particular kit comprises i) (a) a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a distinct therapeutic agent as herein described such as a Selective ER down-regulator/degrader (“SERD”) for example selected from amcenestrant, azenosertib (ZN-c5), borestrant, brilanestrant, camizestrant, elacestrant, fulvestrant, giredestrant, imlunestrant, rintodestrant AZD9496, D-0502 LY3484356, GDC-0927 and SHR9549, and even more preferably elacestrant, in different containers, or a composition comprising the combination of (a) and (b) and a pharmaceutically acceptable carrier, and ii) material(s) for administering compounds (a) and/or (b).

Another particular kit comprises i) (a) a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a distinct therapeutic agent as herein described such as a signal transduction inhibitor such as for example a cyclin-dependent kinase (CDK) inhibitor or a PI3K/Akt/mTOR (“PAM”) pathway inhibitor, in different containers, or a composition comprising the combination of (a) and (b) and a pharmaceutically acceptable carrier, and ii) material(s) for administering compounds (a) and/or (b).

Another particular kit comprises i) (a) a quinazoline carboxamide azetidine compound which is preferably 4-[(S)-2-Azetidin-1-yl-1-(4-chloro-3-trifluoromethyl-phenyl)-ethylamino]-quinazoline-8-carboxylic acid amide (M2698) and/or a pharmaceutically acceptable polymorph, enantiomer, stereoisomer, salt, solvate or tautomer thereof, including any mixture thereof in all ratios, and (b) a distinct therapeutic agent as herein described such as a cyclin-dependent kinase (CDK) inhibitor, preferably a CDK 4/6 inhibitor such as for example abemaciclib, palbocilcib, ribociclib, even more preferably abemaciclib, in different containers, or a composition comprising the combination of (a) and (b) and a pharmaceutically acceptable carrier, and ii) material(s) for administering compounds (a) and/or (b).

When treating or preventing the cancer mentioned herein with a quinazoline carboxamide azetidine compound, in particular M2698, generally satisfactory results are obtained when said compound is administered at a daily dosage of from about 15 milligrams (mg) to about 800 mg, about 50 mg to about 800 mg, or about 15 milligrams (mg) to about 400 mg, possibly given as a single daily dose. The daily dosage of M2698 administered to the subject is for example of about 60 milligrams to about 300 milligrams, preferably of about 80 milligrams to about 280 or 300 milligrams, and even more preferably about 160 milligrams to about 240 milligrams.

In a particular aspect, the quinazoline carboxamide azetidine compound of formula (I) is the 4-[(S)-2-Azetidin-1-yl-1-(4-chloro-3-trifluoromethyl-phenyl)-ethylamino]-quinazoline-8-carboxylic acid amide (“M2698”), and said compound is present in the combination or composition at a dose of about 50 mg to about 800 mg, preferably about 80 mg to about 300 mg, and more preferably about 240 mg.

In particular embodiments, the antineoplastic agent is elacestrant, elacestrant being present in the combination or composition at a dose of about 200 mg to about 500 mg, preferably about 300 mg to about 400 mg, more preferably about 350 mg. Elacestrant is possibly present in said combination or composition as a particular active ingredient, another different active ingredient also present in the combination or composition being the 4-[(S)-2-Azetidin-1-yl-1-(4-chloro-3-trifluoromethyl-phenyl)-ethylamino]-quinazoline-8-carboxylic acid amide (“M2698”) (used as the quinazoline carboxamide azetidine compound).

Other examples of therapeutically effective amounts of a elacestrant or solvates (e.g., hydrate) or salts thereof for use in the methods disclosed herein include, without limitation, about 150 to about 1 500 mg, about 200 to about 1 500 mg, about 250 to about 1 500 mg, or about 300 to about 1 500 mg dosage q.d. for subjects having resistant ER-driven tumors or cancers; about 150 to about 1 500 mg, about 200 to about 1 000 mg or about 250 to about 1 000 mg or about 300 to about 1 000 mg dosage q.d. for subjects having both wild-type ER driven tumors and/or cancers and resistant tumors and/or cancers; and about 300 to about 500 mg, about 300 to about 550 mg, about 300 to about 600 mg, about 250 to about 500 mg, about 250 to about 550 mg, about 250 to about 600 mg, about 200 to about 500 mg, about 200 to about 550 mg, about 200 to about 600 mg, about 150 to about 500 mg, about 150 to about 550 mg, or about 150 to about 600 mg q.d. dosage for subjects having (majorly) wild-type ER driven tumors and/or cancers. In certain embodiments, the dosage of the antineoplastic agent (e.g., elacestrant) or a polymorph, enantiomer, stereoisomer, salt, solvate or tautomer thereof for use in the presently disclosed invention may be approximately 150 mg, 170 mg, 200 mg, 250 mg, 300 mg, 350 mg, 400 mg, 30 mg to 2 000 mg, 100 mg to 1 500 mg, or 150 mg to 1 500 mg, and any amount in between for example 172 mg, 258 mg, or 345 mg, p.o., q.d. for an adult subject. This daily dosage may be achieved via a single administration or multiple administrations.

An effective dosage of the anti-cancer agent, in particular of a small molecule inhibitor, is typically in the range of from about 0.001 to about 100 mg per kg body weight per day, preferably about 1 to about 35 mg/kg/day, in single or divided doses. For a 70 kg human subject, this would amount to about 0.01 to about 7 g/day, preferably about 0.02 to about 2.5 g/day, and more preferably from about 0.02 to about 1.0 g/day. In some instances, dosage levels at the lower limit of the aforesaid range may be more than adequate, while in other cases still larger doses may be employed without causing any harmful side effect, provided that such larger doses are first divided into several small doses for administration throughout the day. The dosage may be administered as a single dose (QD), or optionally may be subdivided into smaller doses, suitable for BID (twice daily), TID (three times daily) or QID (four times daily) administration.

The therapeutically effective amount of a compound depends on a number of factors, including, for example, the age and weight of the animal, the precise condition that requires treatment, and its severity, the nature of the formulation and the method of administration, and is ultimately determined by the treating doctor or vet. However, an effective amount of a compound according to the invention is generally in the range from 0.1 to 100 mg/kg of body weight of the recipient (mammal subject) per day and particularly typically in the range from 1 to 10 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt, solvate, polymorph, tautomer, enantiomer or stereoisomer thereof can be determined as the fraction of the effective amount of the compound according to the invention per se.

In some embodiments, the CDK inhibitor, or a pharmaceutically acceptable polymorph, enantiomer, stereoisomer, salt, solvate or tautomer thereof, is administered at a daily dosage of from about 1 mg to about 1000 mg per day. In some embodiments, the compound or a pharmaceutically acceptable polymorph, enantiomer, stereoisomer, salt, solvate or tautomer thereof, is administered at a daily dosage of from about 10 mg to about 500 mg per day. In some embodiments, it is administered at a dosage of from about 25 mg to about 300 mg per day. In some embodiments, it is administered at dosages of about 1,2, 5, 10, 15, 20, 25, 30, 35, 40,45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220,225, 230, 235, 240, 245, 250, 260, 270, 275, 280, 290, 300, 325, 350, 375, 400, 425, 450, 475 or 500 mg on a QD, BID, TID or QID schedule.

In certain embodiments, the signal transduction inhibitor is a cyclin-dependent kinase (CDK) inhibitor selected for example from a CDK 1, 2, 4, 5, 6 and/or 7 inhibitor and any mixture thereof, more preferably a CDK4/6 inhibitor, and is for example palbociclib, ribociclib or abemaciclib, or a pharmaceutically acceptable polymorph, enantiomer, stereoisomer, salt, solvate or tautomer thereof, which is administered orally at a daily dosage of about 25 mg to about 600 mg per day, and sometimes at a dosage of 25 mg, 50 mg, 75 mg, 100 mg, 125 mg, 150 mg, 200 mg, 250 mg, 300 mg, 350 mg, 400 mg, 450 mg, 500 mg, 550 mg, or 600 mg per day. In other embodiments, the CDK4/6 inhibitor is abemaciclib or a pharmaceutically acceptable polymorph, enantiomer, stereoisomer, salt, solvate or tautomer thereof, which is administered orally at a daily dosage of about 100 mg to about 300 mg per day; or the CDK4/6 inhibitor is palbociclib, or a pharmaceutically acceptable polymorph, enantiomer, stereoisomer, salt, solvate or tautomer thereof, which is administered orally at a daily dosage of about 75 mg to about 125 mg per day; or the CDK4/6 inhibitor is ribociclib, or a pharmaceutically acceptable polymorph, enantiomer, stereoisomer, salt, solvate or tautomer thereof, which is administered orally at a daily dosage of about 200 mg to about 600 mg per day.

This dosage regimen may be adjusted by the oncologist to provide the optimal therapeutic response to the patient.

In a particular example, the quinazoline carboxamide azetidine compound, in particular M2698, can be formulated as an active compound of the combination or a pharmaceutical composition administered orally for example at 80 mg per dose, administered one or several times a day, preferably several times a day, to obtain the desired therapeutic effect. In a preferred aspect of the invention, the patient receives a dose of M2698 of 240 mg/day.

Alternatively, acceptable (pharmaceutical) combinations or compositions described herein may be administered in the form of suppositories for rectal or vaginal administration. These can be prepared by mixing the active compounds of the present application with suitable non-irritating excipients or carriers that are solid at room temperature but liquid at body (e.g. rectal or vaginal) temperature and therefore will melt in the rectum or vaginal cavity to release the active compound(s). Such materials include for example cocoa butter, a suppository wax (e.g., beeswax) and/or polyethylene glycols.

In practical use, the compounds of the present invention can be combined as the active ingredient in intimate admixture with a pharmaceutical carrier according to conventional pharmaceutical compounding techniques. As herein above explained, the carrier may take a wide variety of forms depending on the form of preparation desired for administration, e.g., oral or parenteral (including intravenous).

Pharmaceutically acceptable combinations and compositions described herein may be orally administered in any orally acceptable dosage form including, but not limited to, capsules, tablets, aqueous suspensions or solutions. In such solid dosage forms, the active compound may be admixed with at least one inert diluent such as sucrose, lactose or starch. Such dosage forms may also comprise, as is normal practice, additional substances other than inert diluents, e.g., lubricants and other tableting aids such a magnesium stearate and microcrystalline cellulose. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and/or silicic acid, b) binders such as, for example, carboxymethylcellulose, alginates, gelatin, polyvinylpyrrolidinone, sucrose, and/or acacia, c) humectants such as glycerol, d) disintegrating agents such as agar-agar, calcium carbonate, potato or tapioca starch, alginic acid, certain silicates, and/or sodium carbonate, e) solution retarding agents such as paraffin, f) absorption accelerators such as quaternary ammonium compounds, g) wetting agents such as, for example, cetyl alcohol and/or glycerol monostearate, h) absorbents such as kaolin and/or bentonite clay, and/or i) lubricants such as talc, calcium stearate, magnesium stearate, solid polyethylene glycols, sodium lauryl sulfate, and any mixtures thereof. In the case of capsules, tablets and pills, the dosage form may also comprise buffering agents. The active compound(s) can also be in micro-encapsulated form with one or more excipients as noted above.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings (i.e. buffering agents) and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent (%) of active compound. The percentage of active compound(s) in these compositions may, of course, be varied and may conveniently be between about 2 percent to about 80 percent of the weight of the unit, for example 60% of the weight of the unit. The amount of active compound(s) in such therapeutically useful compositions is such that an effective (i.e., therapeutic) dosage will be obtained. The active compound(s) can also be administered intranasally as, for example, through liquid drops or spray.

Liquid dosage forms for oral administration include, but are not limited to, pharmaceutically acceptable emulsions, microemulsions, solutions, suspensions, syrups and elixirs. In addition to the active compound(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols, and fatty acid esters of sorbitan, as well as any mixtures thereof. Besides inert diluents, the oral compositions can also include adjuvants such as wetting agents, emulsifying and suspending agents, sweetening, flavoring, and perfuming agents.

Compounds of the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oil(s). Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol) and vegetable oils or any suitable mixtures thereof.

Any suitable route of administration may be employed for providing a mammal, especially a human, with an effective dose of a compound of the present invention. For example, oral, rectal, topical, parenteral, ocular, pulmonary, and/or nasal route, and the like may be employed. Dosage forms include tablets, dispersions, suspensions, solutions, capsules, creams, ointments, aerosols, and the like. Preferably, compounds of the present invention are administered orally.

The effective dosage of any active ingredient herein described may vary depending on the particular compound employed, the mode of administration, the condition of treatment and the severity of the condition being treated. Such dosage may be ascertained readily by a person skilled in the art, typically by an oncologist.

### Subject

As explained herein above, in the context of the present invention, the term “patient” or “subject” refer to any single subject for which therapy is desired or that is participating in a clinical trial, epidemiological study or used as a control, including humans and mammalian veterinary patients.

The “subject” or “patient” is typically a mammal. The subject can be a human or a non-human mammal such as a rodent, for example a mouse or a rat, a rabbit, a primate such as a monkey, a dog, a cat, a bovid, an equine, for example a horse, or transgenic species thereof.

In a particular aspect, the mammal is a human being, whatever its age or sex.

In some embodiments, the subject is an adult human subject. The subject is preferably a woman.

In some such embodiments, the subject is a post-menopausal woman or a man.

In some such embodiments, the subject is a pre-menopausal or peri-menopausal woman.

In some such embodiments, the subject is a pre-menopausal or peri-menopausal woman treated with a luteinizing hormone-releasing hormone (LHRH) and/or a gonadotropin releasing hormone (GnRH) agonist such as buserelin, cetrorelix, gonadorelin, goserelin, leuprolide, triptorelin or triptorelix.

In some such embodiments, the subject is a man. In some such embodiments, the subject is a man treated with a luteinizing hormone-releasing hormone (LHRH) and/or a gonadotropin releasing hormone (GnRH) agonist such as goserelin, leuprolide, triptorelin, and degarelix.

In some embodiments, the subject is a human child between the ages of birth and 18 years old.

The subject is typically a subject having a hormone dependent disease, preferably a cancer. Unless otherwise specified in the present disclosure, the cancer is characterized by malignant tumor and/or metastasis present preferably in the brain, a bone, a lung, or the liver.

In a particular aspect, the subject is a child between the ages of birth and 15 years old having a pediatric cancer.

The subject is preferably a subject or patient having a Hormone Receptor positive [“HR+”, for example estrogen receptor+(“ER+”) and/or progesterone receptor+(“PgR+”)]begnin, pre-malignant or malignant tumor, preferably an estrogen receptor-positive (ER+) pre-malignant or malignant tumor.

In a particular aspect, the subject is suffering of a malignant tumor which is ER+, irrespective of her/his Epidermal Growth Factor Receptor-2 status (the tumor or subject may be “HER2positive”, “HER2-negative” or “HER2-low”).

In a preferred embodiment, the subject is suffering of a breast cancer, preferably of an advanced breast cancer, in particular of a metastatic advanced breast cancer.

In a typical aspect, the patient has a histologically and/or cytologically confirmed diagnosis of breast cancer with hormone receptor positive status (ER and/or PgR positive). The patient may have in addition an Epidermal Growth Factor Receptor-2-negative (“HER2-”) status, an Epidermal Growth Factor Receptor-2-positive (“HER2+”) status, or an Epidermal Growth Factor Receptor-2-low (“HER2low”) status as defined by Bergeron A et al., 2023 and Peiffer D. et al., 2023.

In a particular and preferred embodiment of the present invention, the subject is a subject undergoing a treatment of cancer, in particular a conventional treatment of cancer, preferably a hormonal treatment of cancer, or who has undergone such treatment.

This means that, typically, before assessing the sensitivity of the subject to a particular treatment of cancer, or before treating the subject with a compound, combination or composition according to the invention, this subject has been exposed to said particular treatment of cancer. The subject may have been exposed to the complete conventional treatment protocol or to part of a complete conventional treatment protocol, for example to at least one cycle of the total planned treatment protocol. In a preferred embodiment, the subject has been treated with, or exposed to, a drug used in hormonal therapy as herein defined, for example a drug selected from a Selective ER Modulator (SERM), a Selective ER down-regulator Degrader (SERD), an Aromatase Inhibitor (AI) and a Complete Estrogen Receptor Antagonist (“CERAN”).

In a particular aspect, the subject is a subject who has been treated with a drug selected for example from a drug used in hormonal (/endocrine) therapy, a CDK inhibitor, a PI3K/AKT/mTOR (“PAM”) pathway inhibitor, or any combination thereof, in particular the combination of a CDK inhibitor and an hormonal (/endocrine) therapy, the combination of a CDK inhibitor and of a SERD, or the combination of a CDK inhibitor, a SERD and a PI3K/AKT/mTOR (“PAM”) pathway inhibitor.

The subject may be a subject who had exhausted all standard acceptable treatment options, with measurable disease by Response Evaluation Criteria in Solid Tumors (RECIST1.1) criteria (E. A. Eisenhauer, Eur J Cancer. 2009 January; 45(2):228-47) and tumor accessible to biopsy.

The herein above-described subject is preferably a subject or patient having a breast cancer tumor, even more preferably a breast cancer tumor expressing the estrogen receptor alpha (ERa) protein (encoded by the ESR1 gene).

In a preferred aspect, the ESR1 mutational status of the subject is determined on blood circulating tumor deoxyribonucleic acid (ctDNA) using a method known by the skilled person in the art such as the Guardant360 CDx assay before any treatment with a combination or composition of the invention, or during said treatment in order to monitor disease's progression or response of the subject to the treatment. Preferably the ESR1 mutational status is limited to ESR1 missense mutations in the ligand binding domain (between codons 310 to 547).

Patients are preferably treated with the combination or composition of the invention when disease progression and/or an unacceptable therapy induced-toxicity are observed (or detected for example by analyzing the ESR1 mutational status) following a distinct previously applied therapy.

The subject may be a subject who suffers from de novo resistance to hormonal therapy.

The subject is preferably a subject who suffers from an acquired resistance to hormonal therapy.

In a preferred aspect of the invention, the subject is a subject who has developed (acquired) endocrine resistance during or after hormonal/endocrine treatment and whose tumor is characterized by a mutated Estrogen Receptor alpha (ERa). The mutation of ERa is typically responsible for the ER-independent growth of cancer cells or tumor.

In a preferred aspect, the cancer sample or cells obtained from the subject harbors genetic alterations, typically mutation(s) affecting the sequence of the translated protein, and resulting for example in the loss (deletion) of one or more amino acids, in the addition of one or more amino acids, and/or in the substitution of at least one amino acid by another one, in the Estrogen Receptor 1 (ESR1) gene of SEQ ID NO: 3, responsible for the expression of the mutated ERa. In a preferred aspect, the mutation (including for example an addition, a deletion, a substitution or a frame shift mutation) occurs in the ligand-binding domain of the ERa protein wild-type sequence of SEQ ID NO: 1. In a particular aspect, the tumor is characterized by a change in conformation of the ligand-binding domain (SEQ ID NO: 2) of the ERa protein responsible for the constitutive activation of the ER receptor, even in the absence of estrogen.

The subject may be a subject who suffers from a pre-existing mutation in the ESR1 gene, typically of a mutation responsible for the expression of a mutated ERa, said mutation being responsible for the constitutive activation of the ER receptor, even in the absence of estrogen.

Patients' Estrogen Receptor 1 (ESR1) or ERa protein status can be determined by detecting mutations in the ESR1 gene or ERa protein.

Those of skill in the art will recognize that various methods and techniques may be used to determine the ESR1 or ERa status, including sequencing techniques well-known in the art, such as next generation sequencing (NGS) and droplet digital PCR (ddPCR). For example, one useful tool is the Guardant360™, a high sensitivity next-generation sequencing platform (Guardant Health, USA). This diagnostic test can detect any ESR1 mutation with a low sensitivity limit of 0.05%. This tool is for example used in the experimental part for a test performed on liquid tumor biopsy.

Another useful tool is the Sysmex® Inostics Liquid Biopsy (ONCOBEAM™) ctDNA Biomarker standard test (cf. www.sysmex-inostics.com; https://cdn2.hubspot.net/hubfs/5871980/OncoBEAM_ctDNA_Testing_in_Clinical_Practice_NSCLC_web.pdf. Further description and use of this assay can be found at Oxnard, G. R. et al. J. Clin. Oncol. 34(28):3375-3382 (2016); Wu, Y. L. et al. MA08.03 J. Thorac. Oncol. 12, S386 (2017); Mok, T. S. et al. N. Engl. J. Med. 376, 629-640 (2017); and Thress K. et al. Poster presented at: European Society for Medical Oncology 2014 Congress; 2014 Sep. 26-30; Madrid, Spain; #1270P; 25. Murtaza M. et al. Nature. 497, 108-112 (2013)). This diagnostic test can detect any ESR1 mutation with a low level sensitivity limit of 0.05%.

In a preferred aspect of the invention, the ER+ breast cancer is characterized by a mutated Estrogen Receptor alpha (ERa) cancerous tumor wherein the mutation occurs in the ligand-binding domain (i.e. in SEQ ID NO: 2) of the ERa wild-type sequence of SEQ ID NO: 1, is characterized by the change in conformation of the ligand-binding domain (SEQ ID NO: 2) of the ERa protein, and/or is characterized by the expression of ERa in a constitutively active form which does not need the presence or binding of its hormone ligand to be active.

In another preferred aspect of the invention, the ERa mutation can be confirmed by determining a first mutant allele frequency (“MAF”) value of a first ERa mutant equal to or above (>) 0.5% and/or a second MAF value of a second ERa mutant value below (<) 0.5%. The mutant allele frequency or “MAF” is the ratio expressed as a decimal of the number of individual genetic reads bearing a particular mutation relative to a wild-type sequence at a particular location, divided by the total number of individual genetic reads covering the same locus. For example, for a particular sequence position, if the total sequencing depth is 10,000 with an adenine (A) base accounting for 9,900 distinct occurrences, the remaining distinct sequencing occurrences may include, e.g., 23 occurrences having a thymine (T) base in the same position, 42 occurrences having a cytosine (C) base in the same position, and the remaining 35 occurrences having a guanine (G) base in the same position. As the vast majority of sequences have the adenine base in that position (thereby conferring adenine in that position as the “wild type” base), the mutant allele frequency of having a thymine (T) base is calculated as (T)/(T+C+G+A), or here, 23/10000=0.0023. Because the skilled artisan understands that the genetic code is redundant, mutant allele frequency can also be calculated based upon codon coding for a particular amino acid in a particular protein sequence. MAF values reflecting amino acid mutations would therefore group all nucleic acid sequences that encode the same mutation. The skilled artisan also appreciates that a single gene may encode amino acid mutations in different locations. Hence, MAF values may be calculated for multiple amino acid mutations at different locations in a single gene.

In a preferred embodiment, the mutation occurs at at least one residue selected from 380, 392, 404, 422, 463, 536, 537 and 538 of SEQ ID NO: 1, and is preferably an amino acid substitution selected from E380Q, V392I, F404fs, V422del, S463P L536H, L536P, L536Q, L536R, Y537C, Y537D, Y537S, Y537N, D538G even more preferably the Y537S and/or D538G mutation(s).

The skilled artisan will recognize that different ESR1 mutations can result in ERa proteins with various mutations including for example one or more of the following amino acid sequence mutations: E380Q, V392I, F404fs, V422del, S463P, L536H, L536P, L536Q, L536R, Y537C, Y537D, Y537S, Y537N and D538G. In a particular aspect, mutation(s) in the ERa protein sequence at particular positions is/are evaluated in the methods disclosed herein, in particular mutations at amino acid positions 537 and/or 538 in SEQ ID NO: 1 (as opposed to ESR1 genetic mutations in the codons for those positions that have altered nucleic acid sequences but still encode wild type amino acid residues for the given positions within the ERa protein).

In a preferred aspect, the patient is one who has been treated with (exposed to) or who is still treated with a drug used for treating cancer, in particular a drug used in hormonal therapy such as a Selective ER and Modulator (SERM), a Selective ER down-regulator Degrader (SERD), an Aromatase Inhibitor (AI) and/or a Complete Estrogen Receptor Antagonist (“CERAN”), a luteinizing hormone-releasing hormone (LHRH) and/or gonadotropin releasing hormone (GnRH) agonists, for example a Selective ER and Modulator (SERM), a Selective ER down-regulator Degrader (SERD), an Aromatase Inhibitor (AI) and/or a Complete Estrogen Receptor Antagonist (“CERAN”), as further exemplified below. This patient is preferably a subject who does not respond (or in other terms who is resistant) to hormonal therapy.

In an even more preferred aspect, the patient is one who has been treated with (exposed to) or who is still treated with a drug used for treating cancer, in particular a drug selected from a drug used in hormonal (/endocrine) therapy, a CDK inhibitor, a PI3K/AKT/mTOR (“PAM”) pathway inhibitor, and any combination thereof, in particular:
the combination of i) a CDK inhibitor [such as abemaciclib (also known as LY2835219), AG024322, aloisine A, aloisine B, alsterpaullone, aminopurvalanol, AT7519, AZD-5438, AZD5597, BLU-222, BMS-387032, birociclib (XZP 3287), bohemine, butyrolactone, CYC065, dalpiciclib (SHR-6390), dinaciclib, ETH-155008, flavopiridol, FCN-437c, GLR2007, indirubin, indirubin-3′-monoxime, JNJ-7706621, kenpaullone, lerociclib (also known as G1T38), meriolin 3, milciclib, narazaciclib (ON123300), NVP-LCQ19, olomoucine, olomoucine II, palbociclib (also known as Ibrance, PD-0332991 or PF-00080665), PF-07220060, PF-07104091, PF-06873600, PHA-793887, purvalanol A, purvanol B, R-CR8, RGB-286638, RGB286147, ribociclib (also known as LEE-011), riviciclib hydrochloride (P276-00), roniciclib, R-roscovitine, Ro4584820, samuraciclib (also known as CT7001 or ICE0942), SRX-3177, TG02, TQB3303, trilaciclib (also known as GTI128), voruciclib, xylocydin, ZK304709, 10Z-Hymenialdisine, 5-Iodo-indirubin-3′-monoxime and (1R,3S)-3-[3-(([3-(methoxymethyl)-1-methyl-1H-pyrazol-5-yl]carbonyl)amino)-1Hpyrazol-5-yl]cyclopentyl propan-2-ylcarbamate; and related compounds as described in WO2022/018596)], and of ii) an hormonal therapeutic agent [such as a Selective ER Modulator (SERM), a Selective ER down-regulator Degrader (SERD), an Aromatase Inhibitor (AI) inhibitor, or a CERAN, an hormone, a Luteinizing hormone-releasing hormone (LHRH) agonist, a gonadotropin releasing hormone (GnRH) agonist, a progestin, an anti-androgen, a CYP17 inhibitor and an adrenolytic agent], or
the combination of i) a CDK inhibitor, for example any one of the herein above described CDK inhibitor, and of ii) a SERD [such as fulvestrant, elacestrant [(R)-6-(2-(ethyl(4-(2-(ethylamino)ethyl)benzyl)amino)-4-methoxyphenyl)-5, 6,7,8-tetrahydronaphthalen-2-ol, also identified as RAD-1901], amcenestrant [(S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid, also identified as SAR439859], brilanestrant [(E)-3-(4-((E)-2-(2-chloro-4-fluorophenyl)-1-(1H-indazol-5-yl)but-1-en-1-yl)phenyl)acrylic acid, also identified as ARN-810 or GDC-0810], camizestrant [N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine, also identified as AZD9833], giredestrant [3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol, also identified as RG6171 or GDC9545], rintodestrant [(E)-3-(4-((2-(4-fluoro-2,6-dimethylbenzoyl)-6-hydroxybenzo[b]thiophen-3-yl)oxy)phenyl)acrylic acid, also identified as G1T48], AZD9496 [(E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid], (E)-3-(4-((2-(2-(1,1-difluoroethyl)-4-fluorophenyl)-6-hydroxybenzo[b]thiophen-3-yl)oxy)phenyl)acrylic acid (also identified as LSZ102), D-0502, LY3484356, GDC-0927, SHR9549, D0502. In another aspect the SERD may be for example amcenestrant [(S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid, also identified as SAR439859], azenosertib (ZN-c5) [1-[(7R)-7-ethyl-7-hydroxy-5,6-dihydrocyclopenta[b]pyridin-2-yl]-6-[4-(4-methylpiperazin-1-yl)anilino]-2-prop-2-enylpyrazolo[3,4-d]pyrimidin-3-one], borestrant [(7a,17b)-7-[9-[(4,4,5,5,5pentafluoropentyl)Sulfinyl]nonyl]estra-1,3,5(10)-triene-3,17-diol-3 boronic acid], brilanestrant [(E)-3-(4-((E)-2-(2-chloro-4-fluorophenyl)-1-(1H-indazol-5-yl)but-1-en-1-yl)phenyl)acrylic acid, also identified as ARN-810 or GDC-0810], camizestrant [N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine, also identified as AZD9833], elacestrant [(R)-6-(2-(ethyl(4-(2-(ethylamino)ethyl)benzyl)amino)-4-methoxyphenyl)-5,6,7,8-tetrahydronaphthalen-2-ol, also identified as RAD-1901], fulvestrant [(7a,17b)-7-[9-[(4,4,5,5,5pentafluoropentyl)Sulfinyl]nonyl]estra-1,3,5(10)-triene-3,17-diol], giredestrant [3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol, also identified as RG6171 or GDC9545], imlunestrant [(5R)-5-[4-[2-[3-(fluoromethyl)azetidin-1-yl]ethoxy]phenyl]-8-(trifluoromethyl)-5H-chromeno[4,3-c]quinolin-2-ol], rintodestrant [(E)-3-(4-((2-(4-fluoro-2,6-dimethylbenzoyl)-6-hydroxybenzo[b]thiophen-3-yl)oxy)phenyl)acrylic acid, also identified as G1T48], AZD9496 [(E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid], (E)-3-(4-((2-(2-(l,l-difluoroethyl)-4-fluorophenyl)-6-hydroxybenzo[b]thiophen-3-yl)oxy)phenyl)acrylic acid (also identified as LSZ102), D-0502, LY3484356, GDC-0927, or SHR9549], or
the combination of i) a CDK inhibitor, for example any one of the herein above described CDK inhibitor, ii) a SERD, for example any one of the herein above described SERD, and iii) a PI3K/AKT/mTOR (“PAM”) pathway inhibitor [such as bimiralisib, dactolisib tosylate, detorsertib, everolimus, monepantel, omipalisib, onatasertib, ridaforolimus, sapanisertib, sirolimus, Streptomyces sp. OA293, temsirolimus, vitusertib, AL-5880S, AL-58922, AUM-302, CA-102, CA-103, CC-115, CC-223, CT-365, DFN-S29, DHM-ZS, FP-208, FT-I S18, HEC-68498, LXI-15029, ME-344, NSC-765844, OSI-027, OSU-53, OT-043, PQR-514, PTX-367, QR-213, RMC-5552, SN-202, SPR-965 or TAM-03, WXFL-10030390 or XP-105; furesertib, borussertib, capivasertib (AZD5363), celecoxib or a celecoxib derivative, dordaviprone (ONC-201), enzastaurin, ipatasertib, miransertib, uprosertib (GSK2141795), ALM-301, ARQ-751, AT-13148, AZD8055, BAY-1125976, BX795, BX912, COTI-2, DC-120, FXY-1, GSK470, JRP-890, JX06, KS-99, LY-2503029, MK-2206, NISC-6, OSU-03012, PHT-427, PTX-200, RX-0201, RX-0301, SBF1 or TAS-117, alpelisib, buparlisib (BKM120), copanlisib, duvelisib, idelalisib, paxalisib, pictilisib (GDC0941) and sonalisib].

This patient is preferably a subject who does not respond (or in other terms who is resistant) to the treatment.

In a particular aspect, the patient is one who has been exposed, or who is still exposed, to a standard-of-care treatment with an anti-cancer agent and more preferably with an antineoplastic agent and/or a signal transduction inhibitor.

In another particular aspect, the patient is one who has been exposed, or who is still exposed, to a SERD compound such as borestrant, camizestrant and/or fulvestrant, combined with a CDK4/6 inhibitor such as abemaciclib, palbociclib and/or ribociclib.

In another particular aspect, the patient is one who has been exposed, or who is still exposed, to a SERD compound such as camizestrant combined with a CDK4/6 inhibitor such as Palbociclib.

In another particular aspect, the patient is one who has been exposed, or who is still exposed, to a SERD compound such as borestrant, camizestrant, elacestrant and/or fulvestrant combined with a CDK4/6 inhibitor such as abemaciclib, palbociclib and/or ribociclib, and with a PI3K/AKT/mTOR (“PAM”) pathway inhibitor such as alpelisib, capivasertib and/or everolimus.

In another particular aspect, the patient is one who has been exposed, or who is still exposed, to a SERD compound such as fulvestrant combined with a CDK4/6 inhibitor such as abemaciclib, palbociclib and/or ribociclib, and with a PI3K/AKT/mTOR (“PAM”) pathway inhibitor such as capivasertib The treatment may have occurred in a neoadjuvant setting (i.e. before surgery) or not (i.e. after surgery).

In a particular aspect, the patient is a postmenopausal patient with advanced ER+ breast cancer who has been exposed to a SERM such as tamoxifen or to a SERD compound such as borestrant, camizestrant, elacestrant, and/or fulvestrant to an AI such as letrozole, or to a CERAN such as OP-1250, preferably to a SERD compound, in particular to fulvestrant.

In another particular aspect, the patient has been exposed, or is still exposed to, an hormonal agent, preferably a SERD compound such as borestrant, camizestrant, elacestrant and/or fulvestrant, an AI such as letrozole, a CERAN such as OP-1250, or a PI3K/Akt/mTOR (“PAM”) pathway inhibitor.

In a further particular aspect, the patient has been exposed, or is still exposed to, an hormonal therapeutic agent, preferably a SERD compound such as borestrant, camizestrant, elacestrant, and/or fulvestrant, a AI compound such as letrozole, a CERAN compound such as OP-1250, and a CDK4/6i such as palbociclib (PD0332991), ribociclib (LEE011) and/or abemaciclib (LY2835219).

In a further particular aspect, the patient has been exposed, or is still exposed to, a hormonal therapeutic agent, for example fulvestrant, letrozole or lapatinib, preferably a compound such as borestrant, fulvestrant, camizestrant, elacestrant, letrozole, and/or lapatinib.

In another particular aspect, the patient is a premenopausal patient with advanced ER+ breast cancer who has been exposed to ovarian ablation or neutralization (either by oophorectomy, radiotherapy, or by administering luteinizing hormone-releasing hormone antagonist(s)) and to a hormonal therapy involving the administration of a SERM such as tamoxifen.

In another particular aspect, the patient has been exposed, or is still exposed, to an antiestrogen agent in combination with a targeted agent, or in other words an inhibitor of the cell cycle, of the PI3K/Akt/mTOR pathway, of a growth factor receptor; a compound targeting alterations in the ubiquitin-proteasome pathway; or a compound increasing the activity of the bromodomain and/or extra-terminal domain of a protein.

In a further particular aspect, the patient has been successively exposed to identical or different hormonal therapies, preferably different (distinct) independent therapies, in successive sequences/lines of treatment (from 1 line up to 12 lines of treatment with a median of 6 lines of treatment). A particular hormonal treatment can occur several times, i.e., be used in several sequences of the successive sequences of treatment.

### Cancer

As explained herein above (see “definition” section), unless otherwise specified in the present disclosure, the cancer or tumor is a pre-malignant or malignant tumor.

In a preferred aspect of the invention, the cancer is an estrogen receptor-positive (ER+) pre-cancerous, cancerous, pre-malignant or malignant tumor.

In a particular aspect, the pre-malignant or malignant tumor is a ER+ and “mutated ESR1” or “mutated ERa” tumor. This tumor may also be a ER+, mutated ESR1 (or mutated ERa), irrespective of the HER2-tumor status which may be positive, low or negative.

In a particular aspect, the tumor may further have confirmed or potential alterations (mutations) in the BRCA1 and/or BRCA2 genes or proteins.

In another particular aspect, the tumor has confirmed or potential alterations (mutations) in any one of the following genes or proteins: IRS1, PTEN, PIK3CA, AKT1, AKT2, AKT3, mTOR, TSC1, TSC2, EGFR or KRAS.

In a preferred aspect of the invention, the cancer is characterized by malignant tumor and/or metastasis preferably to the brain, a bone, a lung, or the liver, and more especially to the brain.

### Method to Select Patients

Herein described is also a method to select/identify subjects, typically patients, most likely to be sensitive, or more responsive, to cancer treatment, in particular to be rendered sensitive again, or more responsive, to hormonal treatment, after having shown resistance to hormonal therapy. Those subjects are the most likely to benefit of the administration of a compound or composition of the present invention.

This method may be performed in vitro, ex vivo or in vivo, and typically comprises a step of determining if the tumor of the subject is a HR+ tumor, in particular a ER+ tumor, preferably a ER+ and mutated ESR1 tumor (irrespective of the HER2 status of the tumor), and, if confirmed, selecting the subject as likely to be sensitive again to the treatment of cancer, in particular to an hormonal treatment of cancer. The sensitivity, or susceptibility, of a subject to a treatment of cancer indicates whether the subject is “responder” or “non-responder”, in other words whether the subject will or will not, be at least partially treated (tumor growth retardation or regression), preferably be completely treated (cured), by said anti-cancer treatment, as already explained herein above.

The method to select a patient herein described is a predictive method, i.e., a method capable of assessing the ability of a subject to respond in the context of an anticancer treatment as herein defined and not only a prognostic method, only capable of indicating whether the subject will survive to the cancer or die from the cancer.

### Treatment of Cancer

Inventors advantageously herein describe a quinazoline carboxamide azetidine compound, combinations of compounds comprising a quinazoline carboxamide azetidine compound and preferably an antineoplastic agent and/or a signal transduction inhibitor, and pharmaceutical compositions comprising a quinazoline carboxamide azetidine compound, preferably together with an antineoplastic agent and/or a signal transduction inhibitor, and optionally a pharmaceutically acceptable carrier, for use as a medicament, preferably for treating a hormone dependent disease, in particular cancer, preferably an estrogen receptor-positive (ER⁺) cancer (as herein above described) in a subject in need thereof (as herein above described). In a preferred embodiment, the cancer is a breast cancer as herein above described and the subject is preferably a subject as herein above described.

The herein described products (compound(s) of the invention, combination or composition comprising such compound(s)) are preferably for use for treating ER+ breast cancer in a subject who does not respond to endocrine therapy.

Embodiments relate to therapeutic uses in oncology, and corresponding methods of treating cancer, preferably breast cancer, in a subject as herein described, preferably in a patient having a breast cancer tumor expressing the estrogen receptor alpha (ERα) protein (encoded by the ESR1 gene). The ERα protein may be a wild-type or mutated version of ERα.

A particular therapeutic use, or treatment of cancer, involves the use, typically administration, of a quinazoline carboxamide azetidine compound, preferably 4-[(S)-2-Azetidin-1-yl-1-(4-chloro-3-trifluoromethyl-phenyl)-ethylamino]-quinazoline-8-carboxylic acid amide (M2698) or a pharmaceutically acceptable polymorph, enantiomer, stereoisomer, salt, solvate or tautomer thereof, or a pharmaceutical composition comprising said quinazoline carboxamide azetidine compound, and in addition the use of, typically administration, in a subject in need thereof, of a drug selected from an antineoplastic agent, a signal transduction inhibitor and any combination thereof, for example, a Selective ER modulator (SERM) as herein described, a Selective ER down-regulator (SERD) as herein described, an aromatase Inhibitor (AI) as herein described, a Complete Estrogen Receptor Antagonist (“CERAN”) as herein described, an inhibitor of the cell cycle as herein described, a PI3K/Akt/mTOR (“PAM”) pathway inhibitor as herein described, or an inhibitor of the growth factor receptor as herein described, preferably of a drug selected from a SERD, a cyclin-dependent kinase (CDK) inhibitor, a SERM, an AI and a CERAN. In a preferred aspect, the drug is selected from a Selective ER modulator (SERM) as herein described, a Selective ER down-regulator (SERD) as herein described, an aromatase Inhibitor (AI) as herein described, a Complete Estrogen Receptor Antagonist (“CERAN”) as herein described, a cyclin-dependent kinase (CDK) inhibitor as herein described, and a PI3K/Akt/mTOR (“PAM”) pathway inhibitor as herein described.

In a particular aspect of the invention, the compound or composition is administered to the subject after the hormonotherapy first line (therapeutic) treatment step and before any later, typically second, line of treatment involving the administration to the subject of a distinct therapeutic compound used in the treatment of cancer, typically of a HR+ cancer, preferably of breast cancer.

The first step may further include the administration of, for example, a CDK4/6 inhibitor such as palbociclib, ribociclib or abemaciclib.

The later step may include the administration to the subject of a therapeutic compound selected from a SERD agent such as for example elacestrant, and/or fulvestrant; an AI such as exemestane; a PI3K/Akt/mTOR (“PAM”) inhibitor such as for example alpelisib or everolimus; a CERAN such as OP-1250; or a combination of a SERD agent and of a PI3K/Akt/mTOR inhibitor such as for example a combination of everolimus and exemestane.

In another particular aspect, inventors herein disclose a therapeutic use or method of treating a subject suffering from a hormone dependent disease, preferably a cancer, characterized by a mutation of Estrogen Receptor 1 (ESR1), wherein the mutation is, as herein above described, an activation mutation (i.e., it is responsible for the constitutive activation of ESR1). This use/method comprises a step of administering to the subject a quinazoline carboxamide azetidine compound, preferably the 4-[(S)-2-Azetidin-1-yl-1-(4-chloro-3-trifluoromethyl-phenyl)-ethylamino]-quinazoline-8-carboxylic acid amide (M2698) or a pharmaceutically acceptable polymorph, enantiomer, stereoisomer, salt, solvate or tautomer thereof alone, optionally in combination with one or several distinct compounds such as any of the herein described drugs, for example, and without limitation a SERM compound selected from tamoxifen, 4-hydroxytamoxifen, endoxifen, toremifene, droloxifene, idoxifene, raloxifene, arzoxifene, bazedoxifene, pipindoxifene and lasofoxifene; a SERD compound selected from amcenestrant, azenosertib (ZN-c5), borestrant, brilanestrant (ARN-810), camizestrant, elacestrant, fulvestrant, giredestrant, imlunestrant, rintodestrant AZD9496 (LSZ102), D-0502, LY3484356, GDC-0927 or SHR9549, in particular to elacestrant for example from amcenestrant, AZD9496, brilanestrant (ARN-810), camizestrant, D-0502, elacestrant (RAD-1901), fulvestrant, preferably elacestrant, GDC-0927, giredestrant, LY3484356 and rintodestrant; an AI inhibitor selected from aminoglutethimide, anastrozole, exemestane, fadrozole, letrozole and vorozole; and a CERAN compound such as OP-1250; a CDK inhibitor selected from abemaciclib (also known as LY2835219), AG024322, aloisine A, aloisine B, alsterpaullone, aminopurvalanol, AT7519, AZD-5438, AZD5597, BLU-222, BMS-387032, birociclib (XZP 3287), bohemine, butyrolactone, CYC065, dalpiciclib (SHR-6390), dinaciclib, ETH-155008, flavopiridol, FCN-437c, GLR2007, indirubin, indirubin-3′-monoxime, JNJ-7706621, kenpaullone, lerociclib (also known as G1T38), meriolin 3, milciclib, narazaciclib (ON123300), NVP-LCQ19, olomoucine, olomoucine II, palbociclib (also known as Ibrance, PD-0332991 or PF-00080665), PF-07220060, PF-07104091, PF-06873600, PHA-793887, purvalanol A, purvanol B, R-CR8, RGB-286638, RGB286147, ribociclib (also known as LEE-011), riviciclib hydrochloride (P276-00), roniciclib, R-roscovitine, Ro4584820, samuraciclib (also known as CT7001 or ICE0942), SRX-3177, TGO2, TQB3303, trilaciclib (also known as GTI128), voruciclib, xylocydin, ZK304709, 10Z-Hymenialdisine, 5-Iodo-indirubin-3′-monoxime, (1R,3S)-3-[3-(([3-(methoxymethyl)-1-methyl-1H-pyrazol-5-yl]carbonyl)amino)-1Hpyrazol-5-yl]cyclopentyl propan-2-ylcarbamate preferably from a CDK4/6 inhibitor selected from palbocociclib, ribociclib and abemaciclib; and/or a PI3K/Akt/mTOR (“PAM”) pathway inhibitor selected from buparlisib (BKM120), pilaralisib (XL147, SAR245408), pictilisib (GDC-0941), sonolisib (PX-866), dactolisib (BEZ235), sapanisertib (INK128, MLN0128), voxtalisib (XL765, SAR245409), serabelisib (MLN1117), alpelisib (BYL719), perifosine (KRX-0401), MK2206, ipatasertib (GDCO0068), GSK690693, temsirolimus (CCI-779), ridaforolimus (MK8669; deforolimus), sirolimus (rapamycin), everolimus (RAD001), AZD-8055 and OSI-027 (ASP7486). In a particular aspect, the SERD is selected from amcenestrant, azenosertib (ZN-c5), borestrant, brilanestrant, camizestrant, elacestrant, fulvestrant, giredestrant, imlunestrant, rintodestrant AZD9496, D-0502, LY3484356, GDC-0927 or SHR9549; the cyclin-dependent kinase (CDK) inhibitor is preferably a CDK4/6 inhibitor such as for example abemaciclib, palbocociclib or ribociclib; the SERM is selected from tamoxifen and lasofoxifene; the AI is selected from anastrozole and letrozole; or the CERAN is OP-1250.

In a further particular aspect, said therapeutic use or method comprises a step of administering to the subject a quinazoline carboxamide azetidine compound, preferably the 4-[(S)-2-Azetidin-1-yl-1-(4-chloro-3-trifluoromethyl-phenyl)-ethylamino]-quinazoline-8-carboxylic acid amide (M2698) or a pharmaceutically acceptable polymorph, enantiomer, stereoisomer, salt, solvate or tautomer thereof, wherein the amount of the composition comprising M2698 that is administered to the subject once daily is of about 160 mg to about 240 mg, the composition being preferably in the form of a capsule, a tablet, a solution, or a suspension.

In again a further particular aspect, the therapeutic use or method of treating a subject suffering from a hormone dependent disease, preferably a cancer, even more preferably a cancer selected from a breast cancer, an ovarian cancer, a endometrial cancer, in particular a type I endometrial cancer, and/or a cancer that has metastasized preferably to the brain, a bone, a lung, or the liver.

In another particular aspect, inventors herein disclose a therapeutic use or method of treating a subject suffering from a hormone dependent disease, preferably a cancer, characterized by a mutation of Estrogen Receptor 1 (ESR1) as herein described, comprising a step of administering a quinazoline carboxamide azetidine compound, preferably the 4-[(S)-2-Azetidin-1-yl-1-(4-chloro-3-trifluoromethyl-phenyl)-ethylamino]-quinazoline-8-carboxylic acid amide (M2698) or a pharmaceutically acceptable polymorph, enantiomer, stereoisomer, salt, solvate or tautomer thereof, wherein the patient has a (first) mutant allele frequency (“MAF”) value, detected for example on cfDNA in a biological sample, for example blood sample, obtained from a patient, of a first ERa mutant equal to or above (>) 0.5%, wherein said first ERa mutant is preferably Y537S or D538G, and/or a (second) MAF value of ERa mutant below (<) 0.5%, wherein said (second) ERa mutation is preferably D538G, L536H, L536P, L536Q, L536R, Y537C, Y537N, Y537D, Y537S, S463P, E380Q, V392I, F404fs or V422del.

In a further particular aspect, inventors also herein disclose a therapeutic use or method of treating a subject suffering from a hormone dependent disease, preferably a cancer, characterized by a mutation of Estrogen Receptor 1 (ESR1) as herein described, comprising a step of administering a quinazoline carboxamide azetidine compound, preferably the 4-[(S)-2-Azetidin-1-yl-1-(4-chloro-3-trifluoromethyl-phenyl)-ethylamino]-quinazoline-8-carboxylic acid amide (M2698) or a pharmaceutically acceptable polymorph, enantiomer, stereoisomer, salt, solvate or tautomer thereof, wherein the subject has a PgR and/or ER positive status.

The present disclosure also encompasses the advantageous therapeutic use of a quinazoline carboxamide azetidine compound, preferably of 4-[(S)-2-Azetidin-1-yl-1-(4-chloro-3-trifluoromethyl-phenyl)-ethylamino]-quinazoline-8-carboxylic acid amide (M2698) or a pharmaceutically acceptable polymorph, enantiomer, stereoisomer, salt, solvate or tautomer thereof, for treating a metastasized cancer, e.g., a cancer that has spread to the brain, a bone, a lung, the liver, or the central nervous system. As illustrated in table A below, M2698 is able to cross the blood brain barrier (“BBB”) when administered orally at 25 mg/kg of the subject's body weight, i.e. in analogous quantities to that of the Compound 1 defined in WO2021/007146 as an active compound from a cancer that has metastasized to the brain compared to active fulvestrant (which is unable to penetrate the blood-brain barrier).

TABLE A

| | | | Dose | Concentration |
|---|---|---|---|---|
| Matrix | Molecule | Dose | Unit | (ng/ml) |
| Plasma | M2698 | 25 | mg/kg | 1600 |
| Plasma | Compound1 | 10 | mg/kg | 1919 |
| Plasma | Compound1 | 30 | mg/kg | 5558 |
| Brain | fulvestrant | 5 | mg | 501 |
| Brain | Compound1 | 10 | mg/kg | 2147 |
| Brain | Compound1 | 30 | mg/kg | 9708 |
| Brain | M2698 | 25 | mg/kg | 4000 |

Accordingly, the present disclosure provides a therapeutic use or method of treating a subject suffering from a hormone dependent disease, preferably a cancer that has metastasized preferably to the brain, a bone, a lung, the liver or the central nervous system, comprising administering a quinazoline carboxamide azetidine compound of formula I:

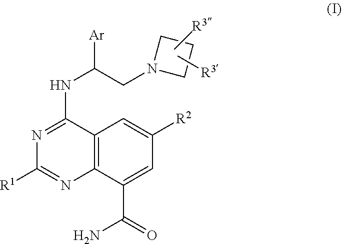

as defined herein, or of formula II:

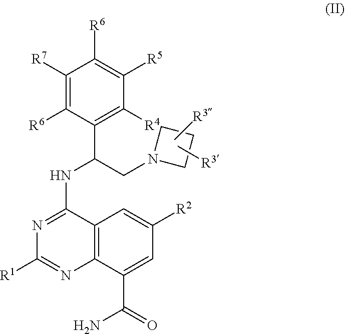

as defined herein preferably the 4-[(S)-2-Azetidin-1-yl-1-(4-chloro-3-trifluoromethyl-phenyl)-ethylamino]-quinazoline-8-carboxylic acid amide (“M2698”) or a pharmaceutically acceptable polymorph, enantiomer, stereoisomer, salt, solvate or tautomer thereof.

In some embodiments, the cancer subject in need of a treatment has one or more CNS metastatic tumors, for example brain metastasis, and optionally in addition metastases in a bone, a lung, or the liver.

All the references cited in this description are incorporated by reference in the present application. Others features and advantages of the invention appear in the following examples and figures which are given for purposes of illustration and not by way of limitation.

### FIGURES

FIG. 1. M2698 combination with elacestrant.

Graph showing the evolution over time (up to 40 days) of the calculated mean volume and standard deviation (SD) of MCF7 tumors in each group: vehicle (n=8 mice); elacestrant alone (n=8 mice); M2698 alone (n=8 mice) and M2698 plus elacestrant (n=6 mice).

FIG. 2. M2698 combination with abemaciclib

Graph showing the evolution over time (up to 40 days) of the calculated mean volume and SD of MCF7 tumors in each group: vehicle (n=8 mice); abemaciblib alone (n=8 mice); M2698 alone (n=8 mice) and M2698 plus abemaciclib (n=6 mice).

FIG. 3. M2698 combination with elacestrant.

Graph showing the evolution over time (up to 60 days) of the calculated mean volume and standard deviation (SD) of MCF7 tumors in each group: vehicle (n=8 mice); elacestrant alone (n=8 mice); M2698 alone (n=8 mice) and M2698 plus elacestrant (n=6 mice). ****p<0.0001, Tukey's range test.

FIG. 4. M2698 combination with abemaciclib

Graph showing the evolution over time (up to 60 days) of the calculated mean volume and SD of MCF7 tumors in each group: vehicle (n=8 mice); abemaciblib alone (n=8 mice); M2698 alone (n=8 mice) and M2698 plus abemaciclib (n=6 mice). ****p<0.0001, Tukey's range test.

### EXAMPLES

The following non-limiting examples are provided to further illustrate certain teachings provided by the present disclosure. Those of skill in the art, in light of the present application, will appreciate that various changes can be made in the specific embodiments that are illustrated in the present Examples without departing from the spirit and scope of the present teachings.

### Example 1

### Materials and Methods

### Patient Cohort Criteria

Female patients aged >18 years old with advanced breast metastatic cancer classified as Hormone receptor positive (HR+, ER+ and/or PgR+) and Epidermal Growth Factor Receptor-2 negative (HER2 neg or HER2−), whose tumors had confirmed or potential alterations in the PAM pathway (PAM+: e.g., PTEN, PIK3CA, AKT1, AKT3, mTOR, TSC1, or TSC2), who had exhausted all standard acceptable treatment options, with measurable disease by Response Evaluation Criteria in Solid Tumors (RECIST1.1) criteria (E. A. Eisenhauer, Eur J Cancer. 2009 January; 45(2):228-47) and tumor accessible to biopsy.

Patients' Estrogen Receptor 1 (ESR1) status was defined by detecting any mutation (including any deletion and/or frame shift) in the ESR1 gene on liquid tumor biopsies in particular at study entry through the Guardant360™, a high sensitivity next-generation sequencing platform (Guardant Health, USA) as retrospective analysis.

Patients with confounding EGFR, KRAS and AKT2 alterations were excluded.

Patients with asymptomatic brain metastases stable for >4 weeks after treatment were eligible.

All patients underwent cardiac function tests for eligibility.

The 26 patients had histologically and/or cytologically confirmed diagnosis of breast cancer with hormone receptor positive status (ER and/or PgR positive) and HER2-negative (“HER2-”) status with prior exposure to tamoxifen and/or an aromatase inhibitor and/or an aromatase inhibitor plus Palbociclib. Prior treatment with tamoxifen in the neoadjuvant setting was allowed but had to have been discontinued for at least 1 year prior to the first dose.

Patients were enrolled in 10 centers in the USA and provided written informed consent before any study procedures were performed. The study was conducted in accordance with ethical principles of the International Council for Harmonization guideline for Good Clinical Practice, the Declaration of Helsinki, and applicable local regulations.

### Drug Substances:

4-[(S)-2-azetidin-1-yl-1-(4-chloro-3-trifluoromethylphenyl)-ethylamino]-quinazoline-8-carboxylic acid amide (“M2698”—CAS no 1379545-95-5) capsule dosing at 80 mg/capsule.
2-[14-[(Z)-1,2-diphenylbut-1-enyl]phenoxy]-N,N-dimethylethanamine (tamoxifen-CAS no 10540-29-1) tablet dosing at 20 mg/tablet.

A list of examples of prior endocrine anti-cancer therapies (ET) that may be administered to patients is described in Table 1 below.

TABLE 1

| | | Treatment | | | | | |
|---|---|---|---|---|---|---|---|
| | | | | | | Tamoxifen | M2698 |
| Patient | Prior | Endocrine | mTor | CDK/6i | | (once | (Once |
| no | Line | Therapy | Inhibitor | inhibitor | Other | daily) | daily) |
| 1 | 7 | tamoxifen, | no | no | | 20 mg | 200 mg |
| | | anastrozole, | | | | | |
| | | fulvestrant | | | | | |
| 2 | 1 | letrozole | no | palbociclib | | 20 mg | 160 mg |
| 3 | 6 | tamoxifen, | everolimus | palbociclib | | 20 mg | 160 mg |
| | | fulvestrant, | | | | | |
| | | letrozole | | | | | |
| 4 | 3 | anastrozole, | everolimus | no | | 20 mg | 80 mg |
| | | fulvestrant, | | | | | |
| | | exemestane | | | | | |
| 5 | 8 | anastrozole, | everolimus | palbociclib | | 20 mg | 160 mg |
| | | exemestane, | | | | | |
| | | fulvestrant | | | | | |
| 6 | 10 | anastrozole, | everolimus | palbociclib | | 20 mg | 160 mg |
| | | tamoxifen, | | | | | |
| | | exemestane, | | | | | |
| | | fulvestrant, | | | | | |
| | | letrozole | | | | | |
| 7 | 9 | letrozole, | everolimus | no | trastuzumab | 20 mg | 240 mg |
| | | tamoxifen, | | | deruxtecan | | |
| | | exemestane | | | | | |
| 8 | 4 | anastrozole, | no | no | | 20 mg | 200 mg |
| | | fulvestrant, | | | | | |
| 9 | 3 | anastrozole, | no | no | | 20 mg | 160 mg |
| | | tamoxifen | | | | | |

ET: Endocrine Therapy includes in particular Aromatase inhibitors (AI), such as letrozole, exemestane and anastrozole as well as Selective Estrogen Receptor Degraders or Modulators (SERD/M) such as tamoxifen and fulvestrant.
Everolimus is the only inhibitor of mTOR (“mTORi”) approved for the treatment of recurrent metastatic ER+ breast cancer.
Palbociclib is one of the 3 inhibitors of the CDK4/6 kinase (CDK4/6i) approved for treatment-naïve advanced ER+ breast cancer. Ribociclib and Abemaciclib could be used similarly. Trastuzumab deruxtecan is an anti-HER2 ADC (antibody drug conjugate).

### Estrogen Receptor 1 (ESR1) Status:

Peripheral blood samples were collected from the patients at study entry. Plasma was isolated from 20 ml whole blood, 20 ng DNA was extracted, and hybrid capture-based genomic profiling of circulating tumor DNA (ctDNA) was carried out in a CLIA-certified/CAP-accredited laboratory (Guardant Health, USA) to identify substitutions, short insertions/deletions, rearrangements/fusions, and amplifications in the ESR1 gene as a component of the multi-gene panel of Guardant360 assay.

### Study Design and Dosing Schedule

The two drug substances were orally administered at the same time, once a day with a full glass of water (approximately 200 mL/8 fl oz) by swallowing tablets whole. Participants took the total assigned dose (one to three capsule(s) of 80 mg of 4-[(S)-2-azetidin-1-yl-1-(4-chloro-3-trifluoromethylphenyl)-ethylamino]-quinazoline-8-carboxylicacid amide and 20 mg of tamoxifen) of in the morning at the same time each day. Participants were instructed to fast for at least 2 hours prior and 1 hour post dosing.

### Statistical Analysis

Progression-free survival (PFS) refers to the time between treatment initiation and tumor progression based on investigator evaluation or death from any cause. Disease progression is defined by the Response Evaluation Criteria in Solid Tumors (RECIST) as an increase in the sum of maximum tumour diameters of at least 20%, the development of any new lesions, or an unequivocal increase in non-measurable malignant disease (E. A. Eisenhauer, Eur J Cancer. 2009 January; 45(2):228-47) and tumor accessible to biopsy. PFS was estimated using the Kaplan-Meier method.

### Results

Among the 26 patients with recurrent ER+ HER2− metastatic breast cancer treated with M2698+tamoxifen:
1 patient received M2698 for only 1 day and was not evaluable for duration of treatment or PFS analyses
6 patients had non evaluable ESR1 status (questionable result due to sample contamination suspicion or absence of data).
19 patients had evaluable ESR1 gene mutational status at study entry.
   9 patients exhibited one or more mutations (Y537D and D538G for around 90%)
   10 patients had unmutated wild-type (WT) ESR1 gene sequence.

TABLE 2

| | | Number of prior | | |
|---|---|---|---|---|
| | Number of | anti-cancer | Duration of | Progression- |
| | evaluable | therapies | Treatment | Free Survival |
| | patients | (median) | (median) | (median) |
| ESR1 | 9 | 6 | 6 months | 5.5 months |
| mutated | | | | |
| ESR1 WT | 10 | 5.5 | 3.17 months | 2.65 months |

Both ESR1 mutated and ESR1 WT patients had very advanced cancer with unfavorable prognosis: both groups had received a median of ~6 prior anti-cancer therapies that had failed. Both were deemed to have exhausted all available therapeutic options to treat their disease. Patients with mutated ESR1 gene stayed on M2698+ tamoxifen combination treatment for around twice as long patients with WT ESR1: median durations of treatment where 6 and 3.17 months, respectively, which means that, in the subgroup of ESRI mutated patients, half of them had disease stabilization lasting longer than 6 months, whereas in the ESRI WT subgroup, half of them had disease stabilization lasting longer than only 3.17 months. Median Progression Free Survival (PFS) in the ESR1 mutated subgroup was nearly twice as long than in the ESR1 WT group. It means that M2698+ tamoxifen delayed disease recurrence for a longer time in the group of patients bearing ESR1 mutations as compared to ESR1 WT: half the patients of the ERS1 mutated group experienced cancer relapse after 5.5 months of treatment, whereas it was only after 2.65 months for the ESR1 WT group.

The provided table 2 illustrates that the patients with ESR1 mutated ER+ breast cancer were unable to respond and had developed resistance to all available therapies, irrespective of their pharmaceutical class and mode of action. Nevertheless, they were able to derive significant clinical benefit (in terms of duration of disease stabilization and time to disease recurrence) when treated with M2698+tamoxifen.

### CONCLUSIONS

Mutations in the ESR1 gene are markers of acquired resistance to Endocrine Therapy of all classes. They are associated with poorer clinical outcome and shorter survival.

The combined use of M2698 compound and tamoxifen, or of M2698 with any other endocrine therapy selected among those herein described, offers an advantageous therapeutic benefit to relapsed/refractory ER+ breast cancer patients having tumors exhibiting mutation(s) in the ESR1 gene.

The M2698 compound can be safely combined with endocrine therapy, the endocrine drug being selected from a Selective ER Modulator (SERM), a Selective ER down-regulator Degrader (SERD), an Aromatase Inhibitor (AI) and a Complete Estrogen Receptor Antagonist (CERAN).

The M2698 compound can be safely combined more specifically with a Selective ER down-regulator Degrader (SERD) selected from amcenestrant, azenosertib (ZN-c5), borestrant, brilanestrant, camizestrant, elacestrant, fulvestrant, giredestrant, imlunestrant, rintodestrant AZD9496 (LSZ102), D-0502, LY3484356, GDC-0927 and SHR9549, and even more specially with elacestrant.

Additionally, the M2698 compound can be safely combined with an endocrine therapeutic compound as herein above described and a compound acting in the ER pathway, the PI3K/Akt/mTOR pathway, a growth factor receptor pathway [involving the epidermal growth factor receptor (EGFR), fibroblast growth factor receptor (FGFR), insulin growth factor receptor (IGFR) or vascular endothelial growth factor receptor (VEGFR)], in the cell cycle, in the ubiquitin-proteasome pathway or in the bromodomain and/or extra-terminal domain of proteins.

The M2698 compound is able to restore the sensitivity of an ER+ tumor to endocrine therapy, which is the cornerstone treatment for ER+ cancers such as for example breast cancer, ovarian cancer, and type I endometrial cancer, preferably for ER+ breast cancers. The ER+ tumor also includes any ER+ tumor that has metastasized to the brain, bone, lung, or liver and more specifically to the brain.

Additionally, the M2698 compound is able to restore the sensitivity of an ESR1 mutated ER+ tumor to endocrine therapy, which is the cornerstone treatment for ER+ cancers such as for example breast cancer, ovarian cancer, and type I endometrial cancer, preferably for ESR1 mutated ER+ breast cancers. The ESR1 mutated ER+ tumor also includes any ESR1 mutated ER+ tumor that has metastasized to the brain, bone, lung, or liver and more specifically to the brain.

### Example 2

Antitumor efficacy of anticancer compounds in a xenograft model of SCID-CB17 mice implanted with MCF7 cells, and assessment of the substrates phosphorylation.

### Materials and Methods

### Experimental Design

MCF7 cells are amplified in vitro in complete medium prior implantation. 24h prior cells injection, the mice are implanted with an estrogen pellet (Innovative Research of America at 0.18 mg/pellet).

On the day of injection, cells are harvested, counted thanks to a trypan blue viability dye (acceptability cut-off ≥80% viability), and resuspended in 50:50 PBS:Matrigel at the appropriate concentration. The cells are then injected subcutaneously in the right flank at 5.10⁶ cells/mouse in 200 μL PBS:Matrigel within 30 minutes after harvesting, in 120 mice.

Mice (female mice, 5 weeks old, specie: SCID-CB17 with a average weight of 20 g (range 18-22 g), are randomized when the tumors reach a mean volume of 100 (+/−25) mm³ for the 8 groups (for a total of 100 mice). After implantation, all the mice are observed in order to detect any toxic effects of the products. The endpoints are defined by animal ethics. They include the assessment of a tumor diameter of >18 mm (1600 mm³), significant weight loss or alteration of animal well-being.

In order to assess the effectiveness of the compounds on tumorigenesis, tumor volume are measured twice per week for 8 weeks and mice are weighed once per week for 8 weeks.

The sizes of the primary tumors are measured using calipers and the tumor volume (TV) is extrapolated to a sphere using the formula TV=4/3π×r3, by calculating the mean radius from the two measurements.

### Treatments

Mice are randomized at tumor uptake and treated daily for 5 weeks, following the treatments indicated in the following Table 3:

TABLE 3

| | No. | | Dose | | Treatment |
|---|---|---|---|---|---|
| Group | Animals | Treatment | (mg/kg) | Route | schedule |
| 1 | 14 | Vehicle (NaCl) | | Per os | qd, 5 weeks |
| 2 | 8 | Vehicle | | Per os | qd, 5 weeks |
| | | M2698 | | | |
| 3 | 8 | Vehicle | | Per os | qd, 5 weeks |
| | | Abemaciclib | | | |
| 4 | 14 | M2698 | 20 | Per os | qd, 5 weeks |
| 5 | 14 | Elacestrant | 50 | Per os | qd, 5 weeks |
| 6 | 14 | Abemaciclib | 40 | Per os | qd, 5 weeks |
| 7 | 14 | M2698 | 20 | Per os | qd, 5 weeks |
| | | Elacestrant | 50 | Per os | qd, 5 weeks |
| 8 | 14 | M2698 | 20 | Per os | qd, 5 weeks |
| | | Abemaciclib | 40 | Per os | qd, 5 weeks |

After 4 days of treatment, tumor sampling of 3 satellite mice 4 h after the last treatment and 3 satellite mice 8 h after the last treatment for 6 groups (Groups 1; 4; 5; 6; 7; 8) for Western Blot analysis (36 total mice). The Western blot assay of 3x2 tumors samples from 6 satellite mice from 6 groups for the semi-quantification of the following proteins will be performed: The whole manipulation is performed on ice. Proteins are extracted using FastPrep fp120 ® (Bio101). Tumors are put in Lysing Matrix D tube with 100 μL of RIPA lysis buffer and are shaken for 12 s at 6.5 m/sec. Then, after 1 h of incubation, they are centrifuged. The supernatant is recovered to be dosed. Lysates are dosed using the Bradford protein assay and adjusted at 50 μg in each sample. Then, each sample is heated at 95° C. for 5 min, loaded on precast gels (Mini-PROTEAN ®, BIO-RAD) to apply a PAGE-SDS. After the end of electrophoresis, protein are transferred using the iBlot ®2 (Thermofisher) with a PVDF Mini Stacks.

### Statistical Analysis

Statistical analysis are performed thanks to GraphPad Prism software. GraphPad Prism combines scientific graphing, comprehensive curve fitting, understandable statistics, and data organization.

Unless specified, the Mann-Whitney t-test (unpaired, two-tailed test) is performed on the tumor volume values (mm³).

### Results

The results are expressed via tumor growth curves.

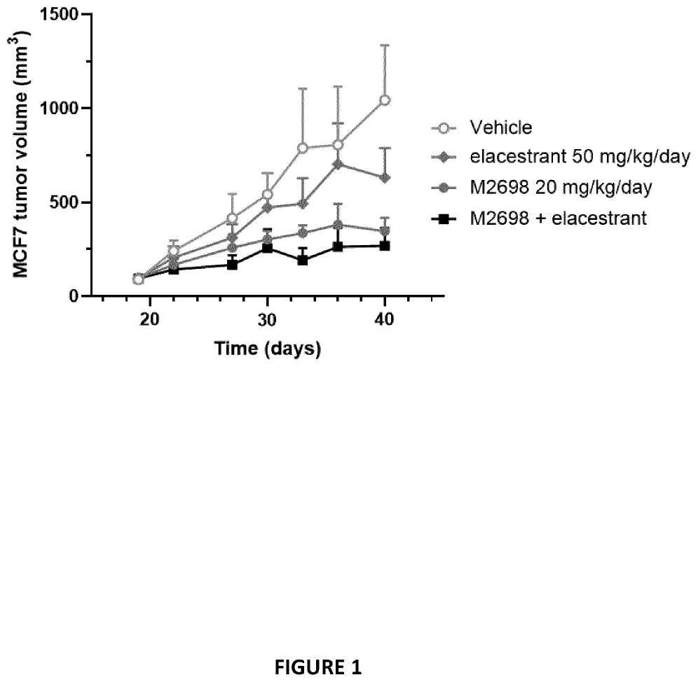

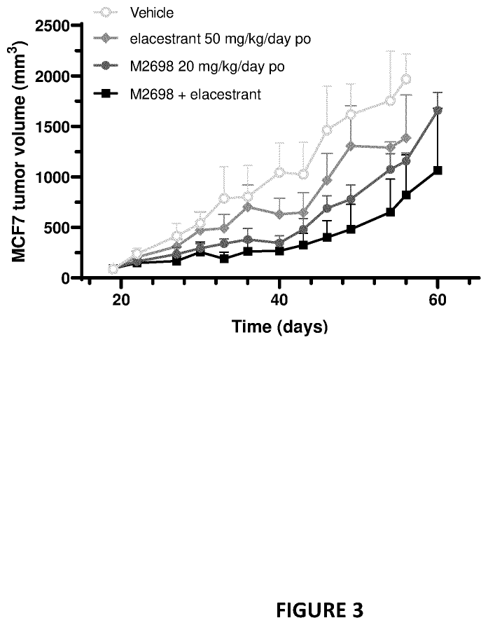

The graphs of FIGS. 1 and 3 show the evolution over time (up to 40 days and up to 60 days respectively) of the calculated mean volume and standard deviation (SD) of MCF7 tumors in each group: vehicle (n=8 mice); elacestrant alone (n=8 mice); M2698 alone (n=8 mice) and M2698 plus elacestrant (n=6 mice).

Elacestrant (50 mg/kg/day) weakly inhibited MCF7 tumor growth, while M2698 (20 mg/kg/day) exhibited strong antitumor efficacy. M2698 and elacestrant combination is significantly more efficient than M2698 alone.

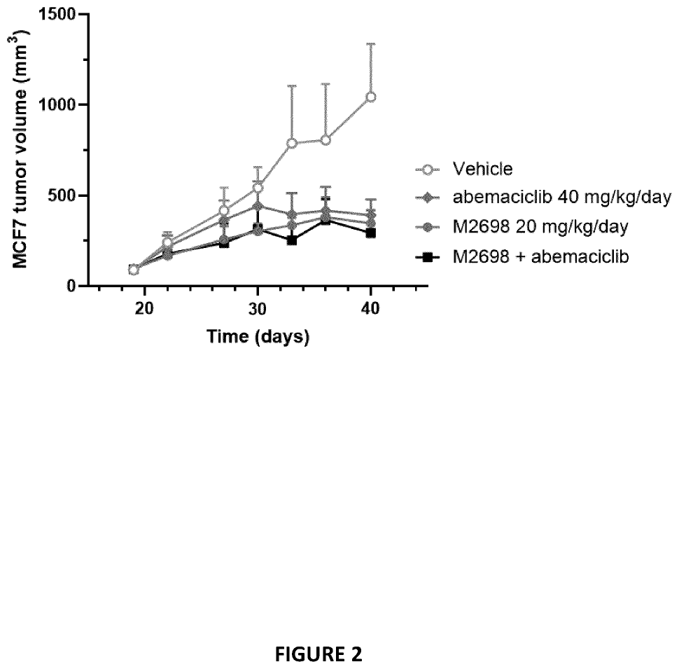

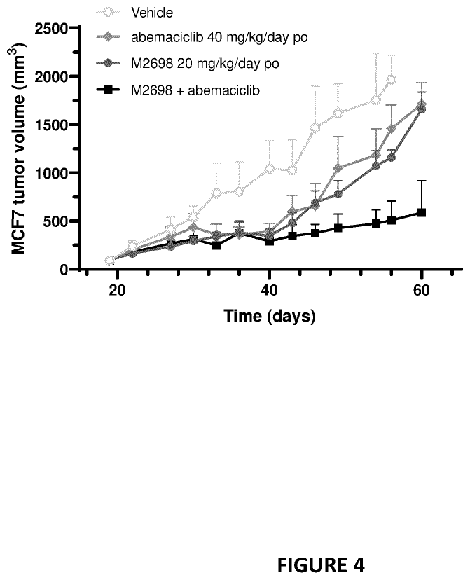

The graphs of FIGS. 2 and 4 show the evolution over time (up to 40 days and up to 60 days respectively) of the calculated mean volume and SD of MCF7 tumors in each group: vehicle (n=8 mice); abemaciclib alone (n=8 mice); M2698 alone (n=8 mice) and M2698 plus abemaciclib (n=6 mice).

M2698 (20 mg/kg/day) strongly inhibited tumor growth, as efficiently as abemaciclib (40 mg/kg/day) alone. The combinations of M2698 and abemaciclib is significantly more efficient than each individual compound.

1-33. (canceled)

34. A combination of (a) a quinazoline carboxamide azetidine compound and (b) a distinct anti-cancer agent selected from an anti-angiogenic agent, a signal transduction inhibitor, an antineoplastic agent, a therapeutic antibody or a fragment thereof, an antibody-drug conjugate, an antisense molecule, a small molecule, a growth factor receptor agent, and any combination thereof, or a composition comprising the combination of (a) and (b) and a pharmaceutically acceptable carrier.

35. The combination or composition according to claim 34, wherein the quinazoline carboxamide azetidine compound is a compound of formula (I): 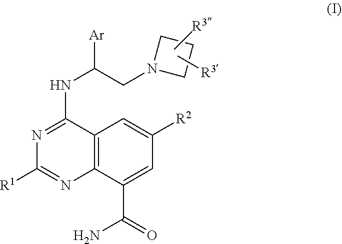
wherein:
R1 is H or LA;
R2 is Hal, O(LA), N(LA)(LA)′, CONH(LA), Ar, CONH2 or A;
R3′, R3″ independently are H, LA or Hal;
Ar is a mono- or bicyclic aromatic homo- or heterocycle having 0, 1, 2, 3 or 4 N, O and/or S atoms and 5, 6, 7, 8, 9, or 10 skeleton atoms, which may be unsubstituted or, independently of one another, mono-, di- or trisubstituted by Hal, A, Art, OH, SH, OA, O(Ar1), NH2, NHA, NH(Ar1), NA2>NO2, CN, OCN, SCN, COOH, COOA, CONH2, CONHA, CONH(Art), CONA2, NHCOA, NHCO(Art), NHCONHA, NHCONH(Art), NHCONH2, NHSO2A, NHSO2(Ar1), COA, CO(Ar1), SO2NH2, SO2A, SO2(Ar1) and/or SO2Hal, and in which a ring N-atom may be substituted by an O-atom to form an N-oxide group, and in which in the case of a bicyclic aromatic cycle on one of the two rings may be partly saturated;
Ar1 is a monocyclic aromatic homo- or heterocycle having 0, 1, 2 or 3 N, O and/or S atoms and 5 or 6 skeleton atoms, which may be unsubstituted or, independently of one another, mono-, di- or trisubstituted by Hal, LA, OH, SH, O(LA), NH2, NH(LA), N(LA)2, NO2, CN, OCN, SCN, COOH, COO(LA), CONH2, CONH(LA), CON(LA)2, NHCO(LA), CHO, CO(LA), SO2NH2, SO2(LA) and/or SO2Hal;
A is unbranched or branched linear or cyclic alkyl having 1, 2, 3, 4, 5, 6, 7 or 8 C atoms, in which one or two CH2 groups may be replaced by an O or S atom and/or by an —NH—, —CO—, —NHCOO—, —NHCONH—, —N(LA)-, —CONH—, —NHCO— or —CH═CH— group, and in which 1-3H atoms may be replaced by Hal, and in which one or two CH3 groups may be replaced by OH, SH, NH2, NH(LA), N(LA)2, NHCOOH, NHCONH2 or CN;
LA is unbranched or branched, linear alkyl having 1, 2, 3 or 4 C atoms, wherein 1, 2 or 3H atoms may be replaced by Hal, methyl, ethyl, trifluoromethyl, difluoromethyl, 1,1,1-trifluoroethyl, propyl, isopropyl, butyl, isobutyl, sec-butyl or tert-butyl; and
Hal is F, CI or Br,
and/or a pharmaceutically acceptable polymorph, enantiomer, stereoisomer, salt, solvate or tautomer thereof, including any mixture thereof in all ratios, and
the distinct therapeutic agent (b) is an antineoplastic agent and/or a signal transduction inhibitor.

36. The combination or composition according to claim 34, wherein the quinazoline carboxamide azetidine compound of formula (I) is 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof.

37. The combination or composition according to claim 34, wherein the distinct therapeutic agent is an antineoplastic agent and said antineoplastic agent is an hormonal therapeutic agent or a chemotherapeutic agent.

38. The combination or composition according to claim 37, wherein the hormonal therapeutic agent is selected from a Selective Estrogen Receptor Degrader (SERD), a Selective Estrogen Receptor Modulator (SERM), an Aromatase Inhibitor (AI) and a Complete Estrogen Receptor Antagonist (CERAN).

39. The combination or composition according to claim 38, wherein the SERD compound is selected from amcenestrant, azenosertib (ZN-c5), borestrant, brilanestrant, camizestrant, elacestrant, fulvestrant, giredestrant, imlunestrant, rintodestrant AZD9496, D-0502, LY3484356, GDC-0927 and SHR9549.

40. The combination or composition according to claim 39, wherein the SERD compound is elacestrant and any pharmaceutically acceptable polymorph, enantiomer, stereoisomer, salt, solvate or tautomer thereof.

41. The combination or composition according to claim 37, wherein the chemotherapeutic agent is selected from an alkylating agent, a platinum coordination complex, a cytotoxic antibiotic, an antimetabolite, a taxane, a topoisomerase inhibitor and a vinca alkaloid.

42. The combination or composition according to claim 37, wherein the chemotherapeutic agent is selected from capecitabin, cyclophosphamide, docetaxel, doxorubicin, epirubicin, eribulin mesylate, fluorouracil, 5-fluorouracil, gemcitabine, liposomal doxorubicin, paclitaxel, vinorelbine, and any pharmaceutically acceptable polymorph, enantiomer, stereoisomer, salt, solvate or tautomer thereof.

43. The combination or composition according to claim 34, wherein the signal transduction inhibitor is a cyclin dependent kinase (CDK) inhibitor.

44. The combination or composition according to claim 43, wherein the CDK inhibitor is a CDK4/6 inhibitor selected from abemaciclib, palbociclib, ribociclib and a pharmaceutically acceptable polymorph, enantiomer, stereoisomer, salt, solvate or tautomer thereof.

45. The combination according to claim 34, wherein the quinazoline carboxamide azetidine compound, the antineoplastic agent and/or the signal transduction inhibitor are formulated for simultaneous, concurrent or sequential administration.

46. A method of treating a hormone dependent disease in a subject comprising administering a combination or composition according to claim 34 to a subject having a hormone dependent disease.

47. The method according to claim 46, wherein:
a) the hormone dependent disease is a hormone receptor-positive (HR+) cancer or an estrogen receptor-positive (ER+) cancer;
b) the hormone dependent disease is a hormone receptor-positive (HR+) cancer or an estrogen receptor-positive (ER+) cancer selected from the group consisting of brain cancer, breast cancer, lung cancer, ovarian cancer, peritoneal cancer, fallopian tube cancer, endometrial cancer, uterine cancer, bladder cancer, colon cancer, prostate cancer, esophageal cancer, liver cancer, pancreatic cancer and stomach cancer;
c) the hormone dependent disease is a hormone receptor-positive (HR+) cancer or an estrogen receptor-positive (ER+) cancer selected from the group consisting of brain cancer, breast cancer, lung cancer, ovarian cancer, peritoneal cancer, fallopian tube cancer, endometrial cancer, uterine cancer, bladder cancer, colon cancer, prostate cancer, esophageal cancer, liver cancer, pancreatic cancer and stomach cancer and the cancer is a human epidermal growth factor receptor 2 positive (HER2+) cancer or a human epidermal growth factor receptor 2 negative (HER2−) cancer or a human epidermal growth factor receptor-2-low (“HER2low”) cancer; or
d) the hormone dependent disease is a hormone receptor-positive (HR+) cancer or an estrogen receptor-positive (ER+) cancer selected from a breast cancer, a metastatic breast cancer, and a metastatic breast cancer resistant and/or refractory to standard-of-care treatment.

48. The method according to claim 47, wherein the treatment involves the administration of an anti-cancer agent selected from an anti-angiogenic agent, a signal transduction inhibitor, an antineoplastic agent, a therapeutic antibody or a fragment thereof, an antibody-drug conjugate, a small molecule, a growth factor receptor agent and/or an antisense molecule.

49. The method according to claim 47, wherein the subject has been treated with a drug selected from a drug used in hormonal therapy, a CDK inhibitor, a PI3K/AKT/mTOR (“PAM”) pathway inhibitor, or any combination thereof.

50. The method according to claim 47, wherein the quinazoline carboxamide azetidine compound of formula (I) is the 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof, and said compound is present in the combination or composition at a dose of about 50 mg to about 800 mg, about 80 mg to about 300 mg or about 240 mg.

51. The method according to claim 48, wherein the antineoplastic agent is elacestrant, elacestrant being present in the combination or composition at a dose of about 200 mg to about 500 mg, about 300 mg to about 400 mg, or about 350 mg.

52. A method of treating estrogen receptor-positive (ER+) cancer in a subject comprising administering a quinazoline carboxamide azetidine compound, or a pharmaceutical composition comprising a quinazoline carboxamide azetidine compound and a pharmaceutically acceptable carrier to the subject.

53. The method according to claim 52, wherein the quinazoline carboxamide azetidine compound is a compound of formula (I): 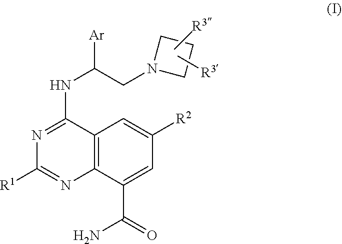
wherein:
R1 is H or LA;
R2 is Hal, O(LA), N(LA)(LA)′, CONH(LA), Ar, CONH2 or A;
R3′, R3″ independently are H, LA or Hal;
Ar is a mono- or bicyclic aromatic homo- or heterocycle having 0, 1, 2, 3 or 4 N, O and/or S atoms and 5, 6, 7, 8, 9, or 10 skeleton atoms, which may be unsubstituted or, independently of one another, mono-, di- or trisubstituted by Hal, A, Art, OH, SH, OA, O(Ar1), NH2, NHA, NH(Ar1), NA2>NO2, CN, OCN, SCN, COOH, COOA, CONH2, CONHA, CONH(Art), CONA2, NHCOA, NHCO(Art), NHCONHA, NHCONH(Art), NHCONH2, NHSO2A, NHSO2(Ar1), COA, CO(Ar1), SO2NH2, SO2A, SO2(Ar1) and/or SO2Hal, and in which a ring N-atom may be substituted by an O-atom to form an N-oxide group, and in which in the case of a bicyclic aromatic cycle on one of the two rings may be partly saturated;
Ar1 is a monocyclic aromatic homo- or heterocycle having 0, 1, 2 or 3 N, O and/or S atoms and 5 or 6 skeleton atoms, which may be unsubstituted or, independently of one another, mono-, di- or trisubstituted by Hal, LA, OH, SH, O(LA), NH2, NH(LA), N(LA)2, NO2, CN, OCN, SCN, COOH, COO(LA), CONH2, CONH(LA), CON(LA)2, NHCO(LA), CHO, CO(LA), SO2NH2, SO2(LA) and/or SO2Hal;
A is unbranched or branched linear or cyclic alkyl having 1, 2, 3, 4, 5, 6, 7 or 8 C atoms, in which one or two CH2 groups may be replaced by an O or S atom and/or by an —NH—, —CO—, —NHCOO—, —NHCONH—, —N(LA)-, —CONH—, —NHCO— or —CH═CH— group, and in which 1-3H atoms may be replaced by Hal, and in which one or two CH3 groups may be replaced by OH, SH, NH2, NH(LA), N(LA)2, NHCOOH, NHCONH2 or CN;
LA is unbranched or branched, linear alkyl having 1, 2, 3 or 4 C atoms, wherein 1, 2 or 3H atoms may be replaced by Hal, methyl, ethyl, trifluoromethyl, difluoromethyl, 1,1,1-trifluoroethyl, propyl, isopropyl, butyl, isobutyl, sec-butyl or tert-butyl; and
Hal is F, CI or Br,
and/or a pharmaceutically acceptable polymorph, enantiomer, stereoisomer, salt, solvate or tautomer thereof, including any mixture thereof in all ratios.

54. The method according to claim 53, wherein the quinazoline carboxamide azetidine compound of formula (I) is 4-[(S)-2-Azetidin-1-yl-1-(4-chloro-3-trifluoromethyl-phenyl)-ethylamino]-quinazoline-8-carboxylic acid amide or a pharmaceutically acceptable polymorph, enantiomer, stereoisomer, salt, solvate or tautomer thereof.

55. The method according to claim 52, wherein:
a) the estrogen receptor-positive (ER+) cancer is selected from brain cancer, breast cancer, lung cancer, ovarian cancer, peritoneal cancer, fallopian tube cancer, endometrial cancer, uterine cancer, bladder cancer, colon cancer, prostate cancer, esophageal cancer, liver cancer, pancreatic cancer and stomach cancer;
b) the estrogen receptor-positive (ER+) cancer is a human epidermal growth factor receptor 2 positive (HER2+) cancer or a human epidermal growth factor receptor 2 negative (HER2−) cancer or a human epidermal growth factor receptor 2 low (HER2 low) cancer;
c) the estrogen receptor-positive (ER+) cancer is a breast cancer, metastatic breast cancer, or a metastatic breast cancer resistant and/or refractory to standard-of-care treatment that is a human epidermal growth factor receptor 2 positive (HER2+) cancer or a human epidermal growth factor receptor 2 negative (HER2−) cancer or a human epidermal growth factor receptor 2 low (HER2 low) cancer.

56. The method according to claim 52, wherein said subject having ER+ breast cancer is a subject who does not respond to hormonal therapy.

57. The method according to claim 52, wherein the estrogen receptor-positive (ER+) cancer is an ER+ breast cancer that is characterized by a mutated Estrogen Receptor alpha (ERα, ERa or ESR1) cancerous tumor, wherein the mutation occurs in the ligand-binding domain of the ERa wild-type sequence of SEQ ID NO: 1, and/or wherein the mutated ERa is characterized by a modification of the conformation of its ligand-binding domain.

58. The method according to claim 57, wherein the mutation occurs on at least one residue selected from residue 380, 392, 404, 422, 463, 536, 537 and 538, or has an amino acid substitution selected from E380Q, V392I, F404fs, V422del, S463P, L536H, L536P, L536Q, L536R, Y537C, Y537D, Y537S, Y537N, and D538G.


SEQUENCE LISTING (3 sequences)

SEQ ID NO: 1 - protein, 595 aa, Homo sapiens [sequence omitted]

SEQ ID NO: 2 - protein, 200 aa, Homo sapiens
KNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQ

SEQ ID NO: 3 - DNA, 2879 bp, Homo sapiens
  source 1..2879: mol_type = genomic DNA
gtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggcc